(19) United States
(12) Patent Application Publication
(10) Pub. No.: US 2024/0197652 A1
(43) Pub. Date: Jun. 20, 2024

(54) KETAMINE IN THE TREATMENT OF BEHAVIORAL ADDICTIONS

(71) Applicant: Awakn LS Europe Holdings Limited, Dublin (IE)

(72) Inventor: Celia Morgan, Exeter (GB)

(21) Appl. No.: 18/563,857

(22) PCT Filed: May 25, 2022
(86) PCT No.: PCT/EP2022/064328

§ 371 Date: Nov. 22, 2023

Related U.S. Application Data
(60) Provisional application No. 63/193,056, filed on May 25, 2021.

(51) Int. Cl. A61K 31/135; A61K 9/00; A61K 9/08; A61K 9/16; A61K 47/02; A61K 47/10; A61K 47/12; A61K 47/20; A61K 47/32; A61K 47/46; A61P 25/00

(52) U.S. Cl. CPC A61K 31/135; A61K 9/0019; A61K 9/0043; A61K 9/0056; A61K 9/0075; A61K 9/08; A61K 9/1623; A61K 47/02; A61K 47/10; A61K 47/12; A61K 47/20; A61K 47/32; A61K 47/46; A61P 25/00

(57) ABSTRACT

Disclosed are pharmaceutical compositions of ketamine and methods of their use in treatment, such as in ketamine-assisted psychotherapy (KAP), including both single KAP sessions and KAP treatment regimes, to treat patients with behavioural addictions, such as gambling disorder, gaming disorder, compulsive sexual behaviour disorder, and binge eating disorder, among others.

### CROSS-REFERENCE

Priority is claimed under PCT Article 8(1) and PCT Rule 4.10 to U.S. Prov. Appl. No. 63/193,056, filed May 25, 2021, and incorporated by reference as if fully set forth herein.

### TECHNICAL FIELD

Described are pharmaceutical compositions of ketamine and methods of their use to treat behavioural addictions such as gambling disorder, gaming disorder, compulsive sexual behaviour disorder, and binge eating disorder, including as part of ketamine-assisted therapy.

### BACKGROUND OF THE INVENTION

Addiction is currently a poorly treated, chronic medical disease involving complex interactions among brain circuits, genetics, the environment, and an individual's life experiences. Addiction is one of the biggest unmet medical needs globally.

People with behavioural addictions engage in behaviours that become compulsive and continue despite harmful consequences. Behavioural addictions like gambling disorder, gaming disorder, compulsive sexual behaviour disorder, and binge eating disorder affect a significant minority of the adults in western societies. Gambling Disorder, Internet Gaming Disorder, and Binge Eating Disorder are all recognised in the DSM-5, and affect up to 450 million, 235 million, and 100 million people respectively, while Compulsive Sexual behaviour Disorder, which is included in the ICD-11, affects up to 350 million people.

Despite the significant prevalence of such behavioural addictions, the current standard of care is poor, and treatment is ineffective.

Psychological interventions in general are ineffective. For instance, a systematic review of problem gambling reviewed cognitive therapies, cognitive behavioural therapy (CBT), motivational interventions, self-directed cognitive behavioural interventions, and feedback interventions, and found none of the interventions offered consistent or persistent changes in gambling in relation to any specific treatment (Petry, Ginley, and Rash, 2017).

Pharmacological interventions are also generally ineffective. To date, the U.S. Food and Drug Administration (FDA) has not approved any drug to treat any behavioural addiction (Ginley et al., 2019). Several medications however have been proposed (see, e.g., Kim, 2001; Olive et al., 2012; Grant, Schreiber & Odlaug 2013; Lupi et al., 2014). One pharmacological approach uses mu-opioid antagonists such as naltrexone. A second approach uses anti-obsessive or antidepressant drugs that affect serotonergic transmission. A third approach uses mood stabilizers such as lithium, atypical antipsychotics, and anticonvulsants.

These pharmacological interventions all have met with limited success, even when paired with psychological treatment. For instance, in a double-blind, placebo-controlled study of naltrexone, 52 patients with gambling disorder and co-occurring alcohol use disorder (AUD) received 7 sessions of CBT (Toneatto, Brands, and Selby, 2009). Both groups responded, and there were no significant differences between naltrexone and placebo. The researchers found that the use of naltrexone was not supported. Clinical trials examining the use of SSRIs (e.g., sertraline, paroxetine, and fluvoxamine) have been mixed. The limitations of the literature also indicate that it is not possible to conclude which antidepressants should be recommended, and which individuals are more likely to respond (Grant, Schreiber, and Odlaug, 2013). Clinical studies of mood stabilising medications (e.g., lithium, topiramate, valproic acid, and carbamazepine) have also delivered mixed results, with some studies failing to prove superiority to placebo in reducing gambling disorder symptoms.

Mindfulness meditation techniques that encourage awareness and acceptance of thoughts, feelings, and bodily sensations also have been explored as a potential approach for behavioural addictions (Shonin, Van Gordon, and Griffiths, 2014; Toneatto, Pillai & Courtice, 2014). However, although this shows promise as an adjunctive treatment to help individuals endure urges, evidence of its effectiveness has been limited, with many individuals needing further cognitive and behavioural skills to cope with high-risk situations for relapse.

Hence, treatment options for behavioural addictions are still sorely needed. Applicant herein discloses one such novel treatment option, and provides a significant solution for the serious clinical, personal, and social problems caused by behavioural addictions.

### INCORPORATION BY REFERENCE

Each patent, publication, and non-patent literature cited in the application or in the section entitled References is hereby incorporated by reference in its entirety as if each was incorporated by reference individually. Reference to any document is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding thereof. This summary is not an extensive overview, nor is it intended to identify every key or critical element of the invention or to delineate the complete scope of the invention. Its sole purpose is to present some exemplary embodiments in a simplified form as a prelude to the more detailed description below.

In some aspects are disclosed methods of treating a patient with a behavioural addiction, by administering to the patient a therapeutically effective amount of ketamine.

In some embodiments, the behavioural addiction is gambling disorder, gaming disorder, compulsive sexual behaviour disorder, compulsive buying-shopping disorder, technology addiction, kleptomania, pyromania, internet addiction, pornography addiction, or binge eating disorder. In some embodiments, the behavioural addiction is classified as mild, moderate, or severe. In some embodiments, the behavioural addiction involves a compulsive or problematic behaviour, and the method results in any of a reduction in the behaviour, a reduction in drive or urge to engage in the behaviour, a promotion of abstinence from the behaviour, a prevention of relapse into the behaviour, or a delay in resumption of the behaviour. In some embodiments, the method results in a reduction in at least one symptom of the behavioural addiction, an elimination of at least one symptom of the behavioural addiction, or a reduction in classification of the behavioural addiction.

In some embodiments, the behavioural addiction is gambling disorder, and the method results in a reduction in gambling, a reduction in drive or urge to engage in gambling, a promotion of abstinence from gambling, a prevention of relapse into gambling, or a delay in resumption of gambling. In embodiments, the reduction in gambling is between 15% and 99%, when measured from baseline. In embodiments, the promotion of abstinence from gambling results in abstinence for at least 1 day, at least 1 week, at least 1 month, or at least 1 year. In embodiments, the prevention of relapse into gambling is for at least 1 day, at least 1 week, at least 1 month, or at least 1 year. In some embodiments, the delay in resumption of gambling is for at least 1 day, at least 1 week, at least 1 month, or at least 1 year.

In some embodiments, the behavioural addiction is gambling disorder, and the method results in a reduction in at least one symptom of the gambling disorder, an elimination of at least one symptom of the gambling disorder, or a reduction in classification of the gambling disorder. In some embodiments, the reduction in at least one symptom of the gambling disorder is a reduction in score of one or more points of the PGSI. In some embodiments, the reduction in at least one symptom of the gambling disorder is a reduction in any of problem gambling and problem gambling expectancy. In some embodiments, the reduction in any of problem gambling and problem gambling expectancy is at least 14%, when measured from baseline. In some embodiments, the reduction in at least one symptom of the gambling disorder or the elimination of at least one symptom of the gambling disorder relates to any of impaired control over gambling; increasing priority given to gambling to the extent that gambling takes precedence over other life interests and daily activities; continuation or escalation of gambling despite the occurrence of negative consequences; needing to gamble with increasing amounts of money in order to achieve the desired excitement; being restless or irritable when attempting to cut down or stop gambling; making repeated unsuccessful efforts to control, cut back, or stop gambling; being preoccupied with gambling, gambling when feeling distressed; returning another day to get even after losing money gambling; lying to conceal the extent of involvement with gambling; jeopardising or losing a significant relationship, job, or educational or career opportunity because of gambling; and reliance on others to provide money to relieve desperate financial situations caused by gambling. In some embodiments, the gambling disorder classified as mild is no longer a mild gambling disorder, the gambling disorder classified as moderate is no longer a moderate gambling disorder, or the gambling disorder classified as severe is no longer a severe gambling disorder.

In some embodiments, the behavioural addiction is gaming disorder and the method results in a reduction in gaming, a reduction in drive or urge to engage in gaming, a promotion of abstinence from gaming, a prevention of relapse into gaming, or a delay in resumption of gaming. In some embodiments, the reduction in gaming is between 15% and 99%, when measured from baseline. In some embodiments, the promotion of abstinence from gaming results in abstinence for at least 1 day, at least 1 week, at least 1 month, or at least 1 year. In some embodiments, the prevention of relapse into gaming is for at least 1 day, at least 1 week, at least 1 month, or at least 1 year. In some embodiments, the delay in resumption of gaming is for at least 1 day, at least 1 week, at least 1 month, or at least 1 year.

In some embodiments, the behavioural addiction is gaming disorder and the method results in a reduction in at least one symptom of the gaming disorder, an elimination of at least one symptom of the gaming disorder, or a reduction in classification of the gaming disorder. In some embodiments, the reduction in at least one symptom of the gaming disorder is a reduction in score of one or more points of any of the IGD-20 and IGDS9-SF. In some embodiments, the reduction in at least one symptom of the gaming disorder is a reduction in craving for gaming of at least 6%, when measured from baseline.

In some embodiments, the reduction in at least one symptom of the gaming disorder or the elimination of at least one symptom of the gaming disorder relates to any of impaired control over gaming behaviour; increasing priority given to gaming behaviour to the extent that gaming takes precedence over other life interests and daily activities; continuation or escalation of gaming behaviour despite negative consequences; preoccupation with gaming; withdrawal symptoms when gaming is taken away or not possible; tolerance; the need to spend more time gaming to satisfy the urge to game; the inability to reduce playing; making unsuccessful attempts to quit gaming; giving up other activities; the loss of interest in previously enjoyed activities due to gaming; continuing to game despite problems; deceiving family members or others about the amount of time spent on gaming; the use of gaming to relieve negative moods, such as guilt or hopelessness; and risking having jeopardised or lost a job or relationship due to gaming. In some embodiments, the gaming disorder classified as mild is no longer a mild gaming disorder, the gaming disorder classified as moderate is no longer a moderate gaming disorder, or the gaming disorder classified as severe is no longer a severe gaming disorder.

In some embodiments, the behavioural addiction is compulsive sexual behaviour disorder and the method results in a reduction in compulsive sexual behaviour, a reduction in drive or urge to engage in compulsive sexual behaviour, a promotion of abstinence from compulsive sexual behaviour, a prevention of relapse into compulsive sexual behaviour, or a delay in resumption of compulsive sexual behaviour. In some embodiments, the reduction in compulsive sexual behaviour is between 15% and 99%, when measured from baseline. In some embodiments, the promotion of abstinence from compulsive sexual behaviour results in abstinence for at least 1 day, at least 1 week, at least 1 month, or at least 1 year. In some embodiments, the prevention of relapse into compulsive sexual behaviour is for at least 1 day, at least 1 week, at least 1 month, or at least 1 year. In some embodiments, the delay in resumption of compulsive sexual behaviour is for at least 1 day, at least 1 week, at least 1 month, or at least 1 year.

In some embodiments, the behavioural addiction is compulsive sexual behaviour disorder and the method results in a reduction in at least one symptom of the compulsive sexual behaviour disorder, an elimination of at least one symptom of the compulsive sexual behaviour disorder, or a reduction in classification of the compulsive sexual behaviour disorder. In some embodiments, the reduction in at least one symptom of the compulsive sexual behaviour disorder is a reduction in score of one or more points of the CSBI-13. In some embodiments, the reduction in at least one symptom of the compulsive sexual behaviour disorder is a reduction in daily hours of pornography viewed of at least 25%, when measured from baseline. In some embodiments, the reduction in at least one symptom of the compulsive sexual behaviour disorder is a reduction in sensitivity to reward of at least 10%, when measured from baseline. In some embodiments, the reduction in at least one symptom of the compulsive sexual behaviour disorder or the elimination of at least one symptom of the compulsive sexual behaviour disorder relates to any of engaging in repetitive sexual behaviour has become a central focus of the patient's life to the point of neglecting health and personal care or other interests, activities and responsibilities; making numerous unsuccessful efforts to control or significantly reduce repetitive sexual behaviour; continuing to engage in repetitive sexual behaviour despite adverse consequences; and continuing to engage in repetitive sexual behaviour even when the individual derives little or no satisfaction from it. In some embodiments, the compulsive sexual behaviour disorder classified as mild is no longer a mild compulsive sexual behaviour disorder, the compulsive sexual behaviour disorder classified as moderate is no longer a moderate compulsive sexual behaviour disorder, or the compulsive sexual behaviour disorder classified as severe is no longer a severe compulsive sexual behaviour disorder.

In some embodiments, the behavioural addiction is compulsive buying-shopping disorder and the method results in a reduction in compulsive buying, a reduction in drive or urge to engage in compulsive buying, a promotion of abstinence from compulsive buying, a prevention of relapse into compulsive buying, or a delay in resumption of compulsive buying. In some embodiments, the reduction in compulsive buying is between 15% and 99%, when measured from baseline. In some embodiments, the promotion of abstinence from compulsive buying results in abstinence for at least 1 day, at least 1 week, at least 1 month, or at least 1 year. In some embodiments, the prevention of relapse into compulsive buying is for at least 1 day, at least 1 week, at least 1 month, or at least 1 year. In some embodiments, the delay in resumption of compulsive buying is for at least 1 day, at least 1 week, at least 1 month, or at least 1 year.

In some embodiments, the method results in a reduction in at least one symptom of the compulsive shopping-buying disorder, an elimination of at least one symptom of the compulsive buying-shopping disorder, or a reduction in classification of the compulsive buying-shopping disorder. In some embodiments, the reduction in at least one symptom of the compulsive buying-shopping disorder is a reduction in score of one or more points of the YBOCS-SV. In some embodiments, the reduction in at least one symptom of the compulsive buying-shopping disorder or the elimination of at least one symptom of the compulsive buying-shopping disorder relates to any of a repeated failure to resist a strong impulse, drive, or urge to perform an act that is rewarding to the patient, at least in the short-term, despite longer-term harm either to the patient or to others; having marked distress about a behaviour pattern; significant impairment in personal, family, social, educational, occupational, or other important areas of functioning; feeling overly preoccupied with shopping and spending; feeling that one's shopping behaviour is excessive, inappropriate or uncontrolled; believing one's shopping desires, urges, fantasies or behaviours are overly time consuming; causing the patient to feel upset or guilty; and causing serious problems in the patient's life including financial or legal problems and relationship loss. In some embodiments, the compulsive buying-shopping disorder classified as mild is no longer a mild compulsive buying-shopping disorder, the compulsive buying-shopping disorder classified as moderate is no longer a moderate compulsive buying-shopping disorder, or the compulsive buying-shopping disorder classified as severe is no longer a severe compulsive buying-shopping disorder.

In some embodiments, the behavioural addiction is technology addiction and the method results in a reduction in compulsive technology use, a reduction in drive or urge to engage in compulsive technology use, a promotion of abstinence from compulsive technology use, a prevention of relapse into compulsive technology use, or a delay in resumption of compulsive technology use. In some embodiments, the reduction in compulsive technology use is between 15% and 99%, when measured from baseline. In some embodiments, the promotion of abstinence from compulsive technology use results in abstinence for at least 1 day, at least 1 week, at least 1 month, or at least 1 year. In some embodiments, the prevention of relapse into compulsive technology use is for at least 1 day, at least 1 week, at least 1 month, or at least 1 year. In some embodiments, the delay in resumption of compulsive technology use is for at least 1 day, at least 1 week, at least 1 month, or at least 1 year.

In some embodiments, the method results in a reduction in at least one symptom of the technology addiction, an elimination of at least one symptom of the technology addiction, or a reduction in classification of the technology addiction. In some embodiments, the reduction in at least one symptom of the technology addiction is a reduction in score of one or more points of the DAS. In some embodiments, the reduction in at least one symptom of the technology addiction or the elimination of at least one symptom of the technology addiction relates to any of an inability to moderate or abstain from technology or a specific digital medium; a preoccupation with thinking about using technological devices; compulsive technological use or experiencing cravings and urges to use digital devices; neglecting important life areas such as work, school or relationships at the expense of technology; continuing to use digital devices despite it contributing to consequences in the patient's life; losing interest in social and leisure activities that the patient once enjoyed at the expense of technology; using digital devices in dangerous situations such as while driving a car or walking across a city street; experiencing unwanted mental health symptoms such as depression, anxiety, stress or irritability at the expense of technological usage; using digital devices to induce pleasure or experience gratification; lying or hiding digital usage from family, friends or colleagues as a result of guilt or shame; and using digital devices for longer durations than intended or finding oneself using digital devices with increased frequency over time. In some embodiments, the technology addiction classified as mild is no longer a mild technology addiction, the technology addiction classified as moderate is no longer a moderate technology addiction, or the technology addiction classified as severe is no longer a severe technology addiction.

In some embodiments, the behavioural addiction is kleptomania and the method results in a reduction in stealing, a reduction in drive or urge to engage in stealing, a promotion of abstinence from stealing, a prevention of relapse into stealing, or a delay in resumption of stealing. In some embodiments, the reduction in stealing is between 15% and 99%, when measured from baseline. In some embodiments, the promotion of abstinence from stealing results in abstinence for at least 1 day, at least 1 week, at least 1 month, or at least 1 year. In some embodiments, the prevention of relapse into stealing is for at least 1 day, at least 1 week, at least 1 month, or at least 1 year. In some embodiments, the delay in resumption of stealing is for at least 1 day, at least 1 week, at least 1 month, or at least 1 year.

In some embodiments, the method results in a reduction in at least one symptom of the kleptomania, an elimination of at least one symptom of the kleptomania, or a reduction in classification of the kleptomania. In some embodiments, the reduction in at least one symptom of the technology addiction is a reduction in score of one or more points of the K-SAS. In some embodiments, the reduction in at least one symptom of the kleptomania or the elimination of at least one symptom of the kleptomania relates to any of a recurrent failure to resist impulses to steal objects not needed for personal use or for monetary value; an increasing sense of tension immediately before committing a theft; pleasure, gratification, or relief at the time of committing the theft; a recurrent failure to control strong impulses to steal objects; a lack of an apparent motive for stealing objects; increased tension or affective arousal prior to instances of theft or attempted theft; and experiencing pleasure, excitement, relief, or gratification during and immediately following the act of stealing. In some embodiments, the kleptomania classified as mild is no longer a mild kleptomania, the kleptomania classified as moderate is no longer a moderate kleptomania, or the kleptomania classified as severe is no longer a severe kleptomania.

In some embodiments, the behavioural addiction is pyromania and the method results in a reduction in fire starting, a reduction in drive or urge to engage in fire starting, a promotion of abstinence from fire starting, a prevention of relapse into fire starting, or a delay in resumption of fire starting. In some embodiments, the reduction in fire starting is between 15% and 99%, when measured from baseline. In some embodiments, the promotion of abstinence from fire starting results in abstinence for at least 1 day, at least 1 week, at least 1 month, or at least 1 year. In some embodiments, the prevention of relapse into fire starting is for at least 1 day, at least 1 week, at least 1 month, or at least 1 year. In some embodiments, the delay in resumption of fire starting is for at least 1 day, at least 1 week, at least 1 month, or at least 1 year.

In some embodiments, the method results in a reduction in at least one symptom of the pyromania, an elimination of at least one symptom of the pyromania, or a reduction in classification of the pyromania. In some embodiments, the reduction in at least one symptom of the pyromania is a reduction in score of one or more points of any of the fire setting scale, fire proclivity scale, and SAFARI. In some embodiments, the reduction in at least one symptom of the pyromania or the elimination of at least one symptom of the pyromania relates to any of deliberate and purposeful fire setting on more than one occasion; tension, or affective arousal before the act; fascination with, interest in, curiosity about, or attraction to fire and its situational contexts; pleasure, gratification, or relief when setting fires or when witnessing or participating in their aftermath; a recurrent failure to control strong impulses to set fires, resulting in multiple acts of, or attempts at, setting fire to property or other objects; a lack of an apparent motive for acts of, or attempts at, fire setting; a persistent fascination or preoccupation with fire and related stimuli; experiencing increased tension or affective arousal prior to instances of, or attempts at, fire setting; and experiencing pleasure, excitement, relief or gratification during, and immediately following the act of setting the fire, witnessing its effects, or participating in its aftermath. In some embodiments, the pyromania classified as mild is no longer a mild pyromania, the pyromania classified as moderate is no longer a moderate pyromania, or the pyromania classified as severe is no longer a severe pyromania.

In some embodiments, the behavioural addiction is internet addiction and the method results in a reduction in compulsive internet use, a reduction in drive or urge to engage in compulsive internet use, a promotion of abstinence from compulsive internet use, a prevention of relapse into compulsive internet use, or a delay in resumption of compulsive internet use. In some embodiments, the reduction in compulsive internet use is between 15% and 99%, when measured from baseline. In some embodiments, the promotion of abstinence from compulsive internet use results in abstinence for at least 1 day, at least 1 week, at least 1 month, or at least 1 year. In some embodiments, the prevention of relapse into compulsive internet use is for at least 1 day, at least 1 week, at least 1 month, or at least 1 year. In some embodiments, the delay in resumption of compulsive internet use is for at least 1 day, at least 1 week, at least 1 month, or at least 1 year.

In some embodiments, the method results in a reduction in at least one symptom of the internet addiction, an elimination of at least one symptom of the internet addiction, or a reduction in classification of the internet addiction. In some embodiments, the reduction in at least one symptom of the internet addiction is a reduction in score of one or more points of any of the IAT, IAS, YDQI, and the CIAS. In some embodiments, the reduction in at least one symptom of the internet addiction or the elimination of at least one symptom of the internet addiction relates to any of a preoccupation with the Internet; need to use the Internet with increased amounts of time in order to achieve satisfaction; repeatedly making unsuccessful efforts to control, cut back, or stop Internet use; being restless, moody, depressed, or irritable when attempting to cut down or stop Internet use; staying online longer than originally intended; having jeopardized or risked a loss of a significant relationship, job, educational or career opportunity because of the Internet; having lied to family members, therapists, or others to conceal the extent of involvement with the Internet; and using the Internet as a way of escaping from problems or of relieving a dysphoric mood. In some embodiments, the internet addiction classified as mild is no longer a mild internet addiction, the internet addiction classified as moderate is no longer a moderate internet addiction, or the internet addiction classified as severe is no longer a severe internet addiction.

In some embodiments, the behavioural addiction is pornography addiction and the method results in a reduction in problematic pornography consumption, a reduction in drive or urge to engage in problematic pornography consumption, a promotion of abstinence from problematic pornography consumption, a prevention of relapse into problematic pornography consumption, or a delay in resumption of problematic pornography consumption. In some embodiments, the reduction in problematic pornography consumption is between 15% and 99%, when measured from baseline. In some embodiments, the promotion of abstinence from problematic pornography consumption results in abstinence for at least 1 day, at least 1 week, at least 1 month, or at least 1 year. In some embodiments, the prevention of relapse into problematic pornography consumption is for at least 1 day, at least 1 week, at least 1 month, or at least 1 year. In some embodiments, the delay in resumption of problematic pornography consumption is for at least 1 day, at least 1 week, at least 1 month, or at least 1 year.

In some embodiments, the method results in a reduction in at least one symptom of the pornography addiction, an elimination of at least one symptom of the pornography addiction, or a reduction in classification of the pornography addiction. In some embodiments, the reduction in at least one symptom of the pornography addiction is a reduction in score of one or more points of any of the BPS and PPCS. In some embodiments, the reduction in at least one symptom of the pornography addiction or the elimination of at least one symptom of the pornography addiction relates to any of being consumed with thoughts of porn even when not actively watching it; viewing porn on a cell phone at work or in social situations where the patient might be seen; a compulsion to watch more pornography; feeling ashamed, guilty, or depressed about porn viewing; continuing to watch porn despite the harm it has had, is having, or may have on relationships, work, or home life; experiencing reduced sexual satisfaction with partners when pornography is not involved; hiding porn and porn viewing from a partner and family members; getting upset when asked to cut back on or stop looking at porn; losing track of time when viewing porn; and trying to quit watching porn but not being successful. In some embodiments, the pornography addiction classified as mild is no longer a mild pornography addiction, the pornography addiction classified as moderate is no longer a moderate pornography addiction, or the pornography addiction classified as severe is no longer a severe pornography addiction.

In some embodiments, the behavioural addiction is binge eating disorder and the method results in a reduction in binge eating, a reduction in drive or urge to engage in binge eating, a promotion of abstinence from binge eating, a prevention of relapse into binge eating, or a delay in resumption of binge eating. In some embodiments, the reduction in binge eating is between 15% and 99%, when measured from baseline. In some embodiments, the promotion of abstinence from binge eating results in abstinence for at least 1 day, at least 1 week, at least 1 month, or at least 1 year. In some embodiments, the prevention of relapse into binge eating is for at least 1 day, at least 1 week, at least 1 month, or at least 1 year. In some embodiments, the delay in resumption of binge eating is for at least 1 day, at least 1 week, at least 1 month, or at least 1 year.

In some embodiments, the method results in a reduction in at least one symptom of the binge eating disorder, an elimination of at least one symptom of the binge eating disorder, or a reduction in classification of the binge eating disorder. In some embodiments, the reduction in at least one symptom of the binge eating disorder is a reduction in score of one or more points of the BES. In some embodiments, the reduction in at least one symptom of the binge eating disorder or the elimination of at least one symptom of the binge eating disorder relates to any of frequent, recurrent episodes of binge eating; feeling like the patient cannot stop or limit the amount or type of food eaten; having difficulty stopping eating once the patient has started; giving up even trying to control eating because the patient knows the patient will end up overeating; marked distress about a pattern of binge eating or significant impairment in personal, family, social, educational, occupational or other important areas of functioning; eating, in a discrete period of time an amount of food that is definitely larger than what most people would eat in a similar period of time under similar circumstances; having a sense of lack of control over eating during an episode; eating much more rapidly than normal, eating until feeling uncomfortably full; eating large amounts of food when not feeling physically hungry; eating alone because of being embarrassed by how much the patient is eating; and feeling disgusted with oneself, depressed, or very guilty after overeating. In embodiments, the binge eating disorder classified as mild is no longer a mild binge eating disorder, the binge eating disorder classified as moderate is no longer a moderate binge eating disorder, or the binge eating disorder classified as severe is no longer a severe binge eating disorder.

In some embodiments, the patient also has a comorbid psychiatric condition. In some embodiments, the comorbid psychiatric condition is any of post-traumatic stress disorder (PTSD), adjustment disorder, affective disorder, depression, atypical depression, postpartum depression, catatonic depression, a depressive disorder due to a medical condition, major depressive disorder (MDD), premenstrual dysphoric disorder, seasonal affective disorder, dysthymia, anxiety, phobia disorders, binge disorders, body dysmorphic disorder, an alcohol or drug abuse or dependence disorder, a substance-related disorder, a substance use disorder, alcohol use disorder, substance-induced mood disorder, a mood disorder related to another health condition, a disruptive behaviour disorder, an eating disorder, an impulse control disorder, obsessive compulsive disorder (OCD), attention deficit hyperactivity disorder (ADHD), a personality disorder, an attachment disorder, and a dissociative disorder. In some embodiments, the methods of the invention are effective to treat the comorbid psychiatric condition. In some embodiments, the methods of the invention result in the reduction in at least one symptom of the comorbid psychiatric condition.

In some aspects herein are ketamine compositions for use in the methods of the invention. In some embodiments, the ketamine is racemic ketamine. In some embodiments, the ketamine is a pure or substantially pure enantiomer of S-ketamine or R-ketamine. In some embodiments, the ketamine is an enantiomerically enriched mixture of ketamine. In some embodiments, the ketamine is norketamine. In some embodiments, the peak plasma concentration of norketamine is between 300 ng/ml and 900 ng/ml after administration to the patient. In some embodiments, the ketamine is hydroxynorketamine. In some embodiments, the peak plasma concentration of hydroxynorketamine is between 50 ng/ml and 650 ng/ml after administration to the patient.

In some embodiments, the ketamine is formulated for intranasal, sublingual, buccal, oral, intramuscular, intravenous, or subcutaneous administration. In some embodiments, the ketamine is formulated for sublingual administration.

In some embodiments, the ketamine is administered in an amount of between about 0.1 mg to about 1.4 mg of ketamine per kilogram of body weight of the patient. In some embodiments, the ketamine is administered in an amount of between about 10 mg to about 125 mg. In some embodiments, the ketamine is administered in an amount of 100 mg. In some embodiments, the ketamine is administered in an amount that is a psycholytic dose. In some embodiments, the psycholytic dose is between about 10 mg to about 40 mg. In some embodiments, the psycholytic dose is between about 0.1 mg to about 0.6 mg per kilogram of body weight of the patient in need thereof. In some embodiments, the ketamine is administered in an amount that is a dissociative dose. In some embodiments, the dissociative dose is between about 50 mg to about 125 mg. In some embodiments, the dissociative dose is between about 0.7 mg to about 1.4 mg per kilogram of body weight of the patient.

In some embodiments, the ketamine compositions of the invention further comprise an additional active agent. In some embodiments, the additional active agent is any of an opioid antagonist a CB-1 antagonist a CRH1 receptor antagonist, a NK1R antagonist, an OTR agonist, a GABA agent, a voltage-gated sodium channel inhibitor, a voltage-dependent calcium channel agonist, an α7 nicotinic acetylcholine receptor agonist, an al adrenoceptor antagonist, a glucocorticoid receptor antagonist, an al adrenoceptor agonist, an AChE inhibitor, a dopamine D2 receptor antagonist, an α2 adrenoceptor agonist, an NMDA receptor antagonist, and an aldehyde dehydrogenase inhibitor, a serotonergic agent, or pharmaceutically acceptable salts thereof.

In some embodiments, the patient is administered the ketamine together with monitoring. In some embodiments, the patient is administered the ketamine together with psychological support. In some embodiments, the patient is administered the ketamine together with psychotherapy. In some embodiments, the psychotherapy includes one or more psychotherapeutic techniques. In some embodiments, the psychotherapy techniques include any of psychosocial or behavioural therapy, cognitive behavioural therapy, interpersonal therapy, contingency management based therapy, community reinforcement approach based therapy, motivational interviewing based therapy, motivational enhancement based therapy, meditation based therapy, acceptance and commitment therapy, dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centred psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, and mindfulness-based cognitive therapy.

In some embodiments, the patient undergoes a ketamine-assisted psychotherapy (KAP) treatment regimen. In some embodiments, the patient undergoing the KAP treatment regimen receives at least one KAP session, and at least one non-drug assisted psychotherapy session. In some embodiments, the at least one session of non-drug assisted psychotherapy session is a preparation session or an integration session. In some embodiments, the KAP treatment regimen takes place over at least four weeks. In some embodiments, the KAP treatment regimen takes place over at least six weeks. In some embodiments, the KAP treatment regimen includes at least two KAP sessions. In some embodiments, the KAP treatment regimen includes at least four KAP sessions.

In some aspects, the methods of the invention are effective to treat a behavioural addiction, or are effective to treat a patient diagnosed with a behavioural addiction.

The foregoing has outlined broadly some pertinent features of certain exemplary embodiments of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific formulations and methods may be readily utilised as a basis for modifying or designing other formulations and methods for carrying out the same purposes of the disclosure. It should be also realised that such equivalent formulations and methods do not depart from the spirit and scope of the invention as set forth in the claims. Hence, this summary is made with the understanding that it will be considered as a brief and general synopsis of only some of the objects and embodiments herein, is provided solely for the benefit and convenience of the reader, and is not intended to limit in any manner the scope, or range of equivalents, to which the claims are lawfully entitled.

It will be appreciated that the headings in this document are utilised only to expedite its review by a reader. They should not be construed as limiting the invention in any manner.

### BRIEF SUMMARY OF THE DRAWINGS

To further clarify various aspects of the invention, a more particular description is rendered by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. Certain aspects of the invention are therefore further described and explained with additional specificity and detail, but still by way of example only, with reference to the accompanying figures in which:

FIG. 1: Illustrates ketamine plasma concentration levels measured in ng/ml in the gambling disorder subjects of EXAMPLE 7 in ten-minute intervals from the time of administration to 90 minutes post-administration.

FIG. 2: Illustrates norketamine and (2S,6S;2R,6R)-hydroxynorketamine (HNK) plasma concentration levels in the gambling disorder subjects of EXAMPLE 7 in ten-minute intervals from the time of administration to 90 minutes post-administration.

FIG. 3: Illustrates the Problem Gambling Severity Index (PGSI) scores and the gambling expectancy scores gathered from the Gambling Related Cognitions Scale (GRCS) in the gambling disorder subjects of EXAMPLE 7 measured prior to undergoing treatment, and two weeks post-administration.

FIG. 4: Illustrates ketamine, HNK, and norketamine plasma concentration levels measured in ng/ml in the binge eating disorder subjects of EXAMPLE 8 in ten-minute intervals from the time of administration to 90 minutes post-administration.

FIG. 5: Illustrates total plasma ghrelin (measured in pg/ml) and total plasma leptin (measured in ng/ml) concentrations in the binge eating disorder subjects of EXAMPLE 8 measured prior to beginning treatment, and at 90 minutes post-lozenge

FIG. 6: Illustrates binge eating scale (BES) scores obtained from the binge eating disorder subjects of EXAMPLE 8 measured prior to beginning treatment, and at the two-week follow up.

FIG. 7: Illustrates ketamine, HNK, and norketamine plasma concentration levels measured in ng/ml in the gaming disorder subjects of EXAMPLE 9 in ten-minute intervals from the time of administration to 90 minutes post-administration.

FIG. 8: Illustrates Craving for Gaming Questionnaire (CGQ) scores obtained from the gaming disorder subjects of EXAMPLE 9 measured prior to beginning treatment, and at the two-week follow up.

FIG. 9: Illustrates ketamine, HNK, and norketamine plasma concentration levels measured in ng/ml in the compulsive sexual behaviour disorder subjects of EXAMPLE 10 in ten-minute intervals from the time of administration to 90 minutes post-administration.

FIG. 10: Illustrates daily hours of pornography viewed by the compulsive sexual behaviour disorder (CSBD) subjects of EXAMPLE 10 and sensitivity to reward (measured by the Sensivity to Reward and Sensitivity to Punishment Scale) of the CSBD subjects of EXAMPLE 11 measured prior to beginning treatment, and at the two-week follow up.

FIG. 11: Illustrates ketamine plasma concentration levels measured in ng/ml in the gambling disorder subjects, binge eating disorder subjects, gaming disorder subjects, and compulsive sexual behaviour disorder subjects of EXAMPLES 7-10 in ten-minute intervals from the time of administration to 90 minutes post-administration.

FIG. 12: Illustrates HNK plasma concentration levels measured in ng/ml in the gambling disorder subjects, binge eating disorder subjects, gaming disorder subjects, and compulsive sexual behaviour disorder subjects of EXAMPLES 7-10 in ten-minute intervals from the time of administration to 90 minutes post-administration.

FIG. 13: Illustrates norketamine plasma concentration levels measured in ng/ml in the gambling disorder subjects, binge eating disorder subjects, gaming disorder subjects, and compulsive sexual behaviour disorder subjects of EXAMPLES 7-10 in ten-minute intervals from the time of administration to 90 minutes post-administration.

FIG. 14: Illustrates mean plasma ketamine, norketamine, and HNK levels measured in ng/ml in the gambling disorder subjects, binge eating disorder subjects, gaming disorder subjects, and compulsive sexual behaviour disorder subjects of EXAMPLES 7-10 in ten-minute intervals from the time of administration to 90 minutes post-administration.

FIG. 15: Illustrates the Depression and Reward Sensitivity scores (using the Beck Depression Inventory (BDI)) of each subject prior to treatment, and at the one-week follow up in the studies completed in EXAMPLES 7-10.

### DETAILED DESCRIPTION

While various aspects and features of certain embodiments are summarised above, the following detailed description illustrates several exemplary embodiments in further detail to enable one having ordinary skill in the art to which the invention belongs (“one of skill”) to practice such embodiments, and to make and use the full scope of the invention claimed.

It will be understood that many modifications, substitutions, changes, and variations in the described examples, embodiments, applications, and details of the invention illustrated herein can be made by those skilled in the art without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and novel features disclosed. The description below is designed to make such embodiments apparent to a one of skill, in that the embodiments shall be both readily cognizable and readily creatable without undue experimentation, solely using the teachings herein together with general knowledge of the art.

The terminology used herein is for describing embodiments and is not intended to be limiting. Herein, the singular forms “a,” “an,” “and,” “the,” and “said” are intended to mean that there are one or more of the elements, and hence include plural referents, unless the content and context clearly dictate otherwise. Thus, for example, a reference to “an excipient” may include a single such excipient, or a combination of two or more excipients.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

The recitation of ranges of values serves as a shorthand for referring individually to each separate value falling within the range. Unless otherwise indicated, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Examples are provided for illustrative purposes, not to limit the scope of the invention or its applications.

In embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless defined otherwise, all technical and scientific terms have the meaning as commonly understood by one of skill. Further definitions to assist the reader in understanding the embodiments are below; however, it will be appreciated that they are not intended to limit the scope of the invention, which shall be properly interpreted and understood by reference to the full specification (as well as any plain meaning known to one of skill) in view of the language used in the appended claims. The terminology used herein is for the purpose of describing particular embodiments only and is not intended to be limiting.

Generally, the nomenclature used and procedures performed herein are those known in fields relating to that of one or more aspects of the invention, such as those of biology, chemistry, pharmacology, neuroscience, psychology, medicine, statistics, and the like, and are those that will be well-known and commonly employed in such fields. Standard techniques and procedures are those generally performed according to conventional methods in the art.

### A. Behavioural Addictions

Herein, “behavioural addictions” generally refers to non-substance addictions that are characterised by an inability to resist an urge to perform a behaviour resulting in actions that are harmful to oneself or others (Grant, Schreiber, & Odlaug, 2013). In embodiments, behavioural addictions are disorders characterised by an intense desire to repeat some action that is pleasurable, or perceived to improve well-being, or capable of alleviating some personal distress, despite the awareness that such an action may have negative consequences.

In some aspects are methods of treating a patient with a behavioural addiction. Herein, a “patient with a behavioural addiction” (alternately, including for all such terms relating to specific disorders, a “behavioural addiction patient”) refers to a patient who may benefit from the methods of treating a behavioural addiction as disclosed herein. In general, the term “patient” may be used interchangeably herein with “subject” and “individual” (e.g., a “subject with a behavioural addiction”). In embodiments, the terms “patient,” “subject,” and “individual” refer to any mammal, although preferably a human, who has an indication for which a composition or method of the invention may be efficacious, or who otherwise may benefit by the invention. In embodiments, as used herein, the terms “patient,” “subject,” and “individual” includes one who has a behavioural addiction, or who has a condition related to a behavioural addiction for which similar treatment may be efficacious. When a “patient,” “subject,” or “individual” is participating in a course of therapy as described below, the term “participant” also may be used. These terms shall refer to individuals in need of treatment for such a disorder, individuals predisposed to such a disorder, and individuals whether or not diagnosed with such a disorder (and irrespective of the system or edition under which they are diagnosed). These terms also shall refer to individuals who have received treatment for a behavioural addiction, who are currently receiving treatment for a behavioural addiction, or who may receive treatment for a behavioural addiction in the future. The term “a subject in need thereof” or “a patient in need thereof” refers to a subject or patient that suffers from, or is at risk for, a behavioural addiction that can be treated according to the disclosed methods.

Examples of behavioural addictions treatable using the disclosed methods include gambling disorder, compulsive sexual behaviour disorder, compulsive buying-shopping disorder, internet addiction, gaming disorder and internet gaming disorder, risk-taking addictions, kleptomania, pyromania, pornography addiction, technology addiction, binge eating disorder, “workaholism,” perfectionism, pathological skin picking, exercise addiction (including overtraining syndrome), excessive tanning, tattoo addiction, masturbation addiction, sex addiction, and love addiction, as well as such other behavioural addictions and disorders as will be known in the art. Different behavioural addictions are often referred to by multiple names, depending on the diagnostic system used (e.g., DSM-5, ICD-11), the edition of that system, and informal use; however, it will be readily appreciated that the specific terminology used herein will not be limiting, and a behavioural addiction may be known, e.g., by its underlying features, signs, and symptoms. Whether behaviour rises to the level of “addiction” generally will depend on whether the behaviour is compulsive and is out-of-control despite adverse consequences (Volk and Lewis, 2015), and whether any such behaviour may constitute a “behavioural addiction” will be ascertainable by those of skill.

Whether a patient has a behavioural addiction can be determined according to ordinary skill. For example, in embodiments, a patient is determined to have a behavioural addiction based on diagnostic criteria provided in the DSM-5, the ICD-11, patient-completed self-assessments, index scores, or any other means known to those of skill.

A behavioural addiction may be characterised as being mild, moderate, and severe. The severity of the behavioural addiction may be determined according to the classification scale of the DSM-5, if applicable, a patient-completed self-assessment, a questionnaire, a measure, an index score, another type of assessment, or otherwise using ordinary skill.

Herein, “reducing” a behaviour or “reduction in” a behaviour refers to reducing the amount or frequency of the behaviour, such as assessed by standardised screening tools like those described herein, and within the knowledge of ordinary skill, and wherein “behaviour” refers to the compulsive or problematic behaviour characteristic of the behavioural addiction. In embodiments, “reducing” a behaviour or “reduction in” a behaviour refers to a reduction in daily engagement in the behaviour, a reduction in the time spent engaging in the behaviour per day, or a reduction in the frequency of engaging in the behaviour, such as a reduction in the percentage of days engaging in the behaviour in general, or a reduction in the percentage of heavy engagement of the behaviour days. In embodiments, “reducing” a behaviour or “reduction in” a behaviour refers to an increase in the time to any engagement in the behaviour or time to the first heavy engagement of the behaviour day. Herein, “heavy engagement” and “heavy” in relation to a compulsive or problematic behaviour will refer to the relative quantity of participation in or performance of the behaviour, as known in the art.

Herein, behavioural “abstinence” or “in abstinence from” a behaviour refers to not engaging in the behaviour. In embodiments, “promoting” behavioural abstinence or “promotion of” behavioural abstinence refers to help maintaining abstinence from the behaviour, in particular after at least 1 day of not engaging in the behaviour, for example, maintaining abstinence from the behaviour for a period of at least 1 day, at least 1 week, at least 1 month, or at least 1 year. Herein, behavioural addiction patient “in abstinence from” a behaviour refers to a patient with a behavioural addiction in abstinence from the behaviour for a period of at least 1 day, at least 1 week, at least 1 month, or at least 1 year.

Herein, the term “at least 1 day, at least 1 week, at least 1 month, or at least 1 year” is shorthand for and as if stating expressly, “at least 1 day, including 1 day, 2 days, 3 days, 4 days, 5 days, and 6 days; for at least 1 week, including 1 week, 2 weeks, and 3 weeks; for at least 1 month, including 1 month, 2 months, 3 months, 4 months, 5 months, and 6 months; for at least 6 months, at least 9 months, or at least 1 year, including for greater than 1 year.”

If not otherwise specified, each time period herein will be measured from baseline.

Herein, “relapse into” a behaviour refers to engaging in a behaviour following a period of abstinence from the behaviour, for example following a period of behavioural abstinence of at least 1 day, at least 1 week, at least 1 month, or at least 1 year. Herein, “preventing relapse into” a behaviour refers to the prevention of engaging in a behaviour by a behavioural addiction patient after the patient has stopped the behaviour, in particular after 1 day or more of not engaging in the behaviour. In embodiments, “1 day or more of not engaging in the behaviour” also encompasses a permanent stoppage of the behaviour. In embodiments, the term encompasses a delay in the resumption of the behaviour as compared to the time to resumption by a patient that is not administered a compound of the invention using the disclosed methods. The delay in resumption can be for at least 1 day, at least 1 week, at least 1 month, or at least 1 year.

In embodiments, a patient with a behavioural addiction is administered ketamine, or a composition comprising ketamine, a pure or substantially pure ketamine enantiomer (i.e., S(+)-ketamine/esketamine or R(−)-ketamine/arketamine), an enantiomerically enriched mixture of ketamine, a ketamine metabolite, or a ketamine analog, or any combination of the foregoing, such as disclosed herein (together, as shorthand, unless context indicates otherwise, “administered ketamine” or “administration of ketamine”). In embodiments, the administration of the ketamine will be effective to treat the behavioural addiction.

In embodiments, a patient with a behavioural addiction is administered ketamine together with psychotherapy (i.e., receives KAP). In embodiments, the administration of the ketamine together with the psychotherapy will be effective to treat the behavioural addiction.

In embodiments, a patient with a behavioural addiction undergoes a KAP treatment regimen. In embodiments, the KAP treatment regimen will be effective to treat the behavioural addiction.

In embodiments, a patient with a behavioural addiction is administered (2S,6S;2R,6R)-hydroxynorketamine (hydroxynorketamine, HNK). In embodiments, the administration of the HNK will be effective to treat the behavioural addiction. In embodiments, a patient with a behavioural addiction is administered HNK together with psychotherapy. In embodiments, the administration of the HNK together with the psychotherapy will be effective to treat the behavioural addiction. In embodiments, a patient with a behavioural addiction undergoes a KAP treatment regimen using HNK. In embodiments, the KAP treatment regimen using HNK will be effective to treat the behavioural addiction. In some embodiments, the HNK will be a single enantiomer of (2S,6S)-HNK or (2R,6R)-HNK, or an enantiomerically enriched mixture thereof.

In embodiments, a patient with a behavioural addiction is administered norketamine. In embodiments, the administration of the norketamine will be effective to treat the behavioural addiction. In embodiments, a patient with a behavioural addiction is administered norketamine together with psychotherapy. In embodiments, the administration of the norketamine together with the psychotherapy will be effective to treat the behavioural addiction. In embodiments, a patient with a behavioural addiction undergoes a KAP treatment regimen using norketamine. In embodiments, the KAP treatment regimen using norketamine will be effective to treat the behavioural addiction.

In embodiments, the administration of ketamine according to the disclosed methods will be effective to treat the behavioural addiction as shown by a reduction in the severity of the behavioural addiction. In embodiments, a patient who has a behavioural addiction characterised as being mild when measured at baseline will no longer have a mild behavioural addiction. In embodiments, a patient who has a behavioural addiction characterised as being moderate when measured at baseline will no longer have a moderate behavioural addiction. In embodiments, a patient who has a behavioural addiction characterised as being severe when measured at baseline will no longer have a severe behavioural addiction.

A reduction in the severity of a behavioural addiction can be determined, e.g., based on an assessment for the behavioural addiction. Exemplary assessments are in TABLE 2.

In embodiments, the administration of ketamine according to the disclosed methods will be effective to treat a behavioural addiction as shown by one or more of: (a) a reduction in the behaviour, (b) a reduction in drive or urge to engage in the behaviour, (c) a promotion of abstinence from the behaviour, (d) a prevention of relapse into the behaviour, (e) a reduction in at least one symptom of the behavioural addiction.

In embodiments, administration of ketamine according to the disclosed methods will result in a measurable reduction in a behaviour or in the drive or urge to engage in the behaviour, for example as compared to a baseline determination made before such administration. In embodiments, the reduction in the behaviour is at least 15%, including 20%, 25%, 30%, 40%, 50%, 60%, 70%, 80%, 90%, up to and including 100%, when measured from baseline. Such a comparison may be made at a time after baseline of, e.g., at least 1 day, at least 1 week, at least 1 month, or at least 1 year.

In embodiments, administration of ketamine according to the disclosed methods will result in a durable promotion of abstinence from the behaviour or prevention of relapse into the behaviour, when measured from the start of treatment, such as the first administration. In embodiments, the promotion of abstinence from the behaviour results in abstinence for at least 1 day, at least 1 week, at least 1 month, or at least 1 year, from the start of treatment. In embodiments, the reduction in the behaviour, reduction in drive or urge to engage in the behaviour, promotion of abstinence from the behaviour, prevention of relapse into the behaviour, and reduction in at least one symptom of the behavioural addiction lasts for at least 1 day, at least 1 week, at least 1 month, or at least 1 year.

In embodiments, administration of ketamine according to the disclosed methods will result in a reduction in at least one symptom of the behavioural addiction, for example as compared to a baseline determination made before such administration. In embodiments, the reduction in at least one symptom of the gambling disorder may be determined by changes in patient completed self-assessments, and clinical outcome assessment or index scores, wherein a decrease in score of one or more points indicates a decrease in the severity of symptoms. In embodiments, administration of ketamine according to the disclosed methods will result in an elimination of at least one symptom of the behavioural addiction, for example as compared to a baseline determination made before such administration.

In some embodiments, a patient with a behavioural addiction, including any one or more of the disorders and conditions herein, also has a comorbid psychiatric condition. Herein, a “comorbid psychiatric condition” may be any of post-traumatic stress disorder (PTSD), adjustment disorder, affective disorder, depression, atypical depression, postpartum depression, catatonic depression, a depressive disorder due to a medical condition, major depressive disorder (MDD), premenstrual dysphoric disorder, seasonal affective disorder, dysthymia, anxiety, phobia disorders, binge disorders, body dysmorphic disorder, alcohol or drug abuse or dependence disorders, substance-related disorders, substance use disorders, alcohol use disorder, substance-induced mood disorder, a mood disorder related to another health condition, disruptive behaviour disorders, eating disorders, impulse control disorders, obsessive compulsive disorder (OCD), attention deficit hyperactivity disorder (ADHD), personality disorders, attachment disorders, and dissociative disorders (see, e.g., Merck Manual of Diagnosis & Therapy, 20th Ed. (2018)).

In embodiments, treating the patient with the behavioural addiction according to the disclosed methods is also effective to treat one or more comorbid psychiatric conditions in the patient. In embodiments, treating the patient with the behavioural addiction will result in a reduction in at least one symptom of a comorbid psychiatric condition. In embodiments, the reduction in at least one symptom of the comorbid psychiatric condition may be a decrease of one or more points of a patient completed self-assessment, as described herein, and/or a decrease of one or more points in a clinical outcome assessment or index score, such as those described in TABLE 2. In embodiments, treating the patient with the behavioural addiction will result in elimination of at least one symptom of a comorbid psychiatric condition. In embodiments, treating the patient with the behavioural addiction will not also be effective to treat one or more comorbid psychiatric conditions in the patient.

a. Gambling Disorder

In some aspects, the methods are useful in treating a patient with gambing disorder. Whether a patient has gambling disorder can be determined according to ordinary skill.

For example, in some embodiments, a patient may be determined to have gambling disorder based on the definition in the 11th Revision of the International Classification of Diseases (ICD-11). The ICD-11 defines Gambling Disorder as being characterised by a pattern of persistent or recurrent gambling behaviour, which may be online (i.e., over the internet) or offline, manifested by: (1) impaired control over gambling (e.g., onset, frequency, intensity, duration, termination, context); (2) increasing priority given to gambling to the extent that gambling takes precedence over other life interests and daily activities; and (3) continuation or escalation of gambling despite the occurrence of negative consequences. The pattern of gambling behaviour may be continuous or episodic and recurrent. The pattern of gambling behaviour results in significant distress or in significant impairment in personal, family, social, educational, occupational or other important areas of functioning. The gambling behaviour and other features are normally evident over a period of at least 12 months in order for a diagnosis to be assigned, although the required duration may be shortened if all diagnostic requirements are met and symptoms are severe.

In other embodiments, a patient may be determined to have gambling disorder based on the definition in the Diagnostic and Statistical Manual of Mental Disorders, 5th Edition (DSM-5). The DSM-5 defines gambling disorder as an addictive disorder in which individuals present with a cluster of symptoms of varying severity that cause psychological and psychical harms and social dysfunction due to their gambling. For diagnosis, four or more of the following symptoms must be present in a 12-month period: (a) needs to gamble with increasing amounts of money in order to achieve the desired excitement, (b) is restless or irritable when attempting to cut down or stop gambling, (c) has made repeated unsuccessful efforts to control, cut back, or stop gambling; (d) is often preoccupied with gambling (e.g., having persistent thoughts of reliving past gambling experiences, handicapping or planning the next venture, thinking of ways to get money with which to gamble), (e) often gambles when feeling distressed (e.g., helpless, guilty, anxious, depressed), (f) after losing money gambling, often returns another day to get even (“chasing” one's losses), (g) lies to conceal the extent of involvement with gambling, (h) has jeopardised or lost a significant relationship, job, or educational or career opportunity because of gambling, and (i) relies on others to provide money to relieve desperate financial situations caused by gambling. Last, the gambling behaviour must not be better explained by a manic episode.

In other embodiments, a gambling disorder patient may have the equivalent underlying disorder, whether that disorder is diagnosed based on the criteria in the ICD-11 or DSM-5, whether the disorder is diagnosed based on a past edition of any system such as the ICD-10 or the DSM-IV (which described pathological gambling as an impulse control disorder with its own specific criteria), whether the disorder is diagnosed based on a future edition of any system, still forthcoming, whether the diagnosis is based on other clinically acceptable criteria, or whether the patient has not yet had a formal clinical diagnosis. It will be readily appreciated that this will be true for all behavioural addictions disclosed herein.

Gambling disorder may be characterised as being mild, moderate, and severe. The severity of gambling disorder may be determined according to the classification scale of the DSM-5, or otherwise using ordinary skill. For example, under the DSM-5, a patient with the presence of four to five symptoms has a mild case, a patient with the presence of six or seven symptoms has a moderate case, and a patient with eight or nine symptoms has a severe case. In embodiments, a patient may have gambling disorder characterised as being mild. In other embodiments, a patient may have a gambling disorder characterised as being moderate. In other embodiments, a patient may have gambling disorder characterised as being severe.

In embodiments, the term “gambling disorder patient” (or “patient with gambling disorder”) refers to a patient diagnosed with gambling disorder, such as defined herein.

Herein, “reducing gambling” or “reduction in gambling” refers to reducing the amount or frequency of gambling, such as assessed by standardised screening tools like the Gambling Timeline Follow-Back (G-TLFB) self-report (Sobell 2001, 1996). In embodiments, “reducing gambling” or “reduction in gambling” refers to a reduction in daily gambling, a reduction in the time spent gambling per day, or a reduction in the frequency of gambling, such as a reduction in the percentage of gambling days in general, or a reduction in the percentage of heavy gambling days. In embodiments, “reducing gambling” or “reduction in gambling” refers to an increase in the time to any gambling or time to the first heavy gambling day.

Herein, “gambling abstinence” or “in abstinence from gambling” refers to not engaging in gambling. In embodiments, “promoting gambling abstinence” or “promotion of gambling abstinence” refers to help maintaining abstinence from gambling, in particular after at least 1 day of not gambling, for example, maintaining abstinence from gambling for a period of at least 1 day, at least 1 week, at least 1 month, or at least 1 year.

Herein, “gambling disorder patient in abstinence from gambling” refers to a patient with gambling disorder in abstinence from gambling for a period, for example, for at least 1 day, at least 1 week, at least 1 month, or at least 1 year.

Herein, “relapse into gambling” refers to engaging in gambling following a period of abstinence from gambling, for example following a period of gambling abstinence of at least 1 day, at least 1 week, at least 1 month, or at least 1 year.

Herein, “preventing relapse into gambling” refers to the prevention of engaging in gambling by a gambling disorder patient after the patient has stopped gambling, in particular after 1 day or more of not gambling. In embodiments, the term encompasses a delay in the resumption of gambling as compared to the time to resumption by a patient that is not administered a compound of the invention using the disclosed methods. The delay in resumption can be for at least 1 day, at least 1 week, at least 1 month, or at least 1 year.

In embodiments, the patient with gambling disorder is administered ketamine. In embodiments, the patient with gambling disorder receives KAP. In embodiments, the patient with gambling disorder undergoes a KAP treatment regimen. In embodiments, the administration of the ketamine is effective to treat gambling disorder. In embodiments, the KAP is effective to treat gambling disorder. In embodiments, the KAP treatment regimen is effective to treat gambling disorder. In embodiments, HNK or norketamine is administered. In embodiments, HNK or norketamine is effective to treat gambling disorder.

In embodiments, the administration of ketamine according to the disclosed methods will be effective to treat gambling disorder as shown by a reduction in the severity of gambling disorder. In embodiments, a patient who has gambling disorder characterised as being mild when measured at baseline will no longer have a mild gambling disorder. In embodiments, a patient who has gambling disorder characterised as being moderate when measured at baseline will no longer have a moderate gambling disorder. In embodiments, a patient who has gambling disorder characterised as being severe when measured at baseline will no longer have a severe gambling disorder.

A reduction in the severity of gambling disorder can be determined based on an assessment for Gambling Disorder. In some embodiments, the assessment for Gambling Disorder is the Problem Gambling Severity Index (PGSI). In embodiments, a patient who scores 1-2 on the PGSI when measured at baseline will have a reduction in PGSI score of at least 1 point after treatment with the disclosed methods. In embodiments, a patient who scores 3-7 on the PGSI when measured at baseline will have a reduction in PGSI score of at least 3 points after treatment with the disclosed methods. In embodiments, a patient who scores 8 or more on the PGSI when measured at baseline will have a reduction in PGSI score of at least 5 points after treatment with the disclosed methods.

In some embodiments, the assessment for Gambling Disorder is any of the Lie-Bet Tool to Rule Out Problem Gambling, the National Opinion Research Center Diagnostic Screen for Gambling Disorders, Loss of Control, Lying, and Preoccupation screen (NODS-Clip), the National Opinion Research Center Diagnostic Screen for Gambling Disorders preoccupation, Escape, Risked Relationships And Chasing Screen (NODS-PERC), the Brief Bio-Social gambling Screen (BBGS), the DSM-V Screen for Pathological Gambling, the NORC Diagnostic Screen, the South Oaks Gambling Screen, and the South Oaks Gambling Screen revised for adolescents.

In embodiments, the administration of ketamine according to the disclosed methods will be effective to treat gambling disorder as shown by one or more of: (a) a reduction in gambling, (b) a reduction in drive or urge to engage in gambling, (c) a promotion of abstinence from gambling, (d) a prevention of relapse into gambling, (e) a reduction in at least one symptom of gambling disorder.

In embodiments, administration of ketamine according to the disclosed methods will result in a measurable reduction in gambling or in the drive or urge to engage in gambling, for example as compared to a baseline determination made before such administration. In embodiments, the reduction in gambling is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from gambling or prevention of relapse into gambling, when measured from the start of treatment, such as the first administration. In embodiments, the promotion of abstinence from gambling results in abstinence for at least 1 day, at least 1 week, at least 1 month, or at least 1 year, from the start of treatment. In embodiments, the reduction in gambling, reduction in drive or urge to engage in gambling, promotion of abstinence from gambling, prevention of relapse into gambling, and reduction in at least one symptom of gambling disorder lasts for at least 1 day, at least 1 week, at least 1 month, or at least 1 year.

In embodiments, administration of ketamine according to the disclosed methods will result in a reduction in at least one symptom of gambling disorder, for example as compared to a baseline determination made before such administration. In embodiments, the reduction in at least one symptom of gambling disorder may be determined by changes in patient completed self-assessments, and clinical outcome assessment or index scores, wherein a decrease in score of one or more points indicates a decrease in the severity of symptoms. In embodiments, the clinical outcome assessment or index includes the PGSI. In embodiments, administration of ketamine according to the disclosed methods will result in an elimination of at least one symptom of gambling disorder, for example as compared to a baseline determination made before such administration, and where elimination means that the patient no longer has the symptom or meets the criteria therefor, and which may be for a period of time of, for example, at least 1 day, at least 1 week, at least 1 month, or at least 1 year.

In embodiments, administration of ketamine according to the disclosed methods will result in the patient having a reduction in at least one symptom of gambling disorder, including impaired control over gambling, increasing priority given to gambling to the extent that gambling takes precedence over other life interests and daily activities, continuation or escalation of gambling despite the occurrence of negative consequences, needs to gamble with increasing amounts of money in order to achieve the desired excitement, is restless or irritable when attempting to cut down or stop gambling, has made repeated unsuccessful efforts to control, cut back, or stop gambling; is often preoccupied with gambling, often gambles when feeling distressed, after losing money gambling, often returns another day to get even; lies to conceal the extent of involvement with gambling, has jeopardised or lost a significant relationship, job, or educational or career opportunity because of gambling; and relies on others to provide money to relieve desperate financial situations caused by gambling.

In embodiments, administration of ketamine according to the disclosed methods will result in the patient having an elimination of at least one symptom of a gambling disorder, including impaired control over gambling, increasing priority given to gambling to the extent that gambling takes precedence over other life interests and daily activities, continuation or escalation of gambling despite the occurrence of negative consequences, needs to gamble with increasing amounts of money in order to achieve the desired excitement, is restless or irritable when attempting to cut down or stop gambling, has made repeated unsuccessful efforts to control, cut back, or stop gambling; is often preoccupied with gambling, often gambles when feeling distressed, after losing money gambling, often returns another day to get even; lies to conceal the extent of involvement with gambling, has jeopardised or lost a significant relationship, job, or educational or career opportunity because of gambling; and relies on others to provide money to relieve desperate financial situations caused by gambling.

In embodiments, the patient with gambling disorder has a comorbid psychiatric condition. In embodiments, treating the patient with gambling disorder is also effective to treat one or more comorbid psychiatric conditions in the patient.

b. Gaming Disorder

In some aspects, the methods are useful in treating a patient with gaming disorder. Whether a patient has gaming disorder can be determined according to ordinary skill. For example, in some embodiments, a patient may be determined to have gaming disorder based on the definition in the ICD-11. The ICD-11 defines Gaming Disorder (GD) as a pattern of gaming behaviour, for example, “digital-gaming” or “video-gaming.”

Exemplary criteria include (1) a persistent pattern of gaming behaviour (‘digital gaming’ or ‘video-gaming’), which may be predominantly online (i.e., over the internet or similar electronic networks) or offline, manifested by all of the following: (a) impaired control over gaming behaviour (e.g., onset, frequency, intensity, duration, termination, context); (b) increasing priority given to gaming behaviour to the extent that gaming takes precedence over other life interests and daily activities, (c) and continuation or escalation of gaming behaviour despite negative consequences (e.g., family conflict due to gaming behaviour, poor scholastic performance, negative impact on health); (2) the pattern of gaming behaviour may be continuous or episodic and recurrent but is manifested over an extended period of time (e.g., 12 months); (3) the gaming behaviour is not better accounted for by another mental disorder (e.g., Manic Episode) and is not due to the effects of a substance or medication; (4) the pattern of gaming behaviour results in significant distress or impairment in personal, family, social, educational, occupational, or other important areas of functioning (ICD-11). For a diagnosis, the behaviour must be of sufficient severity to result in significant impairment in personal, family, social, educational, occupational or other important areas of functioning and would normally have been evident for at least 12 months (WHO, “Addictive behaviours: Gaming disorder,” 2020). In other embodiments, a patient may be determined to have a gaming disorder based on questionnaires known to those of skill, such as the Gaming Disorder Scale for Adolescents (GADIS-A) (Paschke et al., J. Clin. Med., 2020; 9(4), 993).

In embodiments, the Internet Gaming Disorder Scale (IGD-20) and Internet Gaming Disorder Scale-Short Form (IGDS9-SF) may be utilised to distinguish disordered and non-disordered gaming, wherein scores of 71 or above on the IGD-20 and 32 points or above on the IGDS9-SF may be considered disordered, and scores below those values are considered non-disordered.

In some embodiments, a patient may be determined to have gaming disorder based on the DSM-5, which includes Internet gaming disorder (IGD) as a non-substance addiction. Nine criteria for IGD were recommended in the DSM-5, where such gaming also causes “significant impairment or distress” in several aspects of the patient's life: (a) preoccupation with gaming, (b), withdrawal symptoms when gaming is taken away or not possible (sadness, anxiety, irritability), (c) tolerance, the need to spend more time gaming to satisfy the urge, (d) inability to reduce playing, unsuccessful attempts to quit gaming, (e) giving up other activities, loss of interest in previously enjoyed activities due to gaming, (f) continuing to game despite problems, (g) deceiving family members or others about the amount of time spent on gaming, (h) the use of gaming to relieve negative moods, such as guilt or hopelessness, (i) risk, having jeopardised or lost a job or relationship due to gaming. Under the proposed criteria, a diagnosis of internet gaming disorder would require experiencing five or more of these symptoms within a year. The condition can include gaming on the internet, or on any electronic device, although most people who develop clinically significant gaming problems play primarily on the internet.

In embodiments, the term “gaming disorder patient” (or “patient with gambing disorder”) refers to a patient diagnosed with a gaming disorder, such as defined herein.

Herein, “reducing gaming” or “reduction in gaming” refers to reducing the amount or frequency of gaming, such as assessed by standardised screening tools like the IGD-20 and IGDS9-SF. In embodiments, “reducing gaming” or “reduction in gaming” refers to a reduction in daily gaming, a reduction in the time spent gaming per day, or a reduction in the frequency of gaming, such as a reduction in the percentage of gaming days in general, or a reduction in the percentage of heavy gaming days. In embodiments, “reducing gaming” or “reduction in gaming” refers to an increase in the time to any gaming or time to the first heavy gaming day.

Herein, “gaming abstinence” or “in abstinence from gaming” refers to not engaging in gaming. In embodiments, “promoting gaming abstinence” or “promotion of gaming abstinence” refers to help maintaining abstinence from gaming, in particular after at least 1 day of not gaming, for example, maintaining abstinence from gaming for a period of at least 1 day, at least 1 week, at least 1 month, or at least 1 year.

Herein, “gaming disorder patient in abstinence from gaming” refers to a patient with gaming disorder in abstinence from gaming for a period, for example, for at least 1 day, at least 1 week, at least 1 month, or at least 1 year.

Herein, “relapse into gaming” refers to engaging in gaming following a period of abstinence from gaming, for example following a period of gaming abstinence of at least 1 day, at least 1 week, at least 1 month, or at least 1 year.

Herein, “preventing relapse into gaming” refers to the prevention of engaging in gaming by a gaming disorder patient after the patient has stopped gaming, in particular after 1 day or more of not gaming. In embodiments, the term encompasses a delay in the resumption of gaming as compared to the time to resumption by a patient that is not administered a compound of the invention using the disclosed methods. The delay in resumption can be for at least 1 day, at least 1 week, at least 1 month, or at least 1 year.

In embodiments, the patient with gaming disorder is administered ketamine. In embodiments, the patient with gaming disorder receives KAP. In embodiments, the patient with gaming disorder undergoes a KAP treatment regimen. In embodiments, the administration of the ketamine is effective to treat gaming disorder. In embodiments, the KAP is effective to treat gaming disorder. In embodiments, the KAP treatment regimen is effective to treat gaming disorder. In embodiments, HNK or norketamine is administered. In embodiments, HNK or norketamine is effective to treat gaming disorder.

In embodiments, the administration of ketamine according to the disclosed methods will be effective to treat gaming disorder as shown by a reduction in the severity of gaming disorder. In embodiments, a patient who has gaming disorder characterised as being mild when measured at baseline will no longer have a mild gaming disorder. In embodiments, a patient who has gaming disorder characterised as being moderate when measured at baseline will no longer have a moderate gaming disorder. In embodiments, a patient who has gaming disorder characterised as being severe when measured at baseline will no longer have a severe gaming disorder A reduction in the severity of gaming disorder can be determined based on ordinary skill, such as using an assessment.

In embodiments, the administration of ketamine according to the disclosed methods will be effective to treat gaming disorder as shown by reducing a patient classified as a disordered gamer pre-treatment to a classification as a non-disordered gamer post-treatment. In embodiments, a patient who scores 71 points or higher on the IGD-20 when measured at baseline will have a reduction in the IGD-20 score of at least 10 points. In embodiments, a patient who scores 71 points or higher on the IGD-20 when measured at baseline will have a post-treatment IGD-20 score of less than 71 points. In embodiments, a patient who scores 32 points or higher on the IGDS9-SF when measured at baseline will have a reduction in the IGDS9-SF score of at least 5 points. In embodiments, a patient who scores 32 points or higher on the IGD-20 when measured at baseline will have a post-treatment IGD-20 score of less than 32 points.

In embodiments, the administration of ketamine according to the disclosed methods will be effective to treat gaming disorder as shown by one or more of: (a) a reduction in gaming, (b) a reduction in drive or urge to engage in gaming, (c) a promotion of abstinence from gaming, (d) a prevention of relapse into gaming, (e) a reduction in at least one symptom of gaming disorder.

In embodiments, administration of ketamine according to the disclosed methods will result in a measurable reduction in gaming or in the drive or urge to engage in gaming, for example as compared to a baseline determination made before such administration. In embodiments, the reduction in gaming is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from gaming or prevention of relapse into gaming, when measured from the start of treatment, such as the first administration. In embodiments, the promotion of abstinence from gaming results in abstinence for at least 1 day, at least 1 week, at least 1 month, or at least 1 year, from the start of treatment. In embodiments, the reduction in gaming, reduction in drive or urge to engage in gaming, promotion of abstinence from gaming, prevention of relapse into gaming, and reduction in at least one symptom of gaming disorder lasts for at least 1 day, at least 1 week, at least 1 month, or at least 1 year.

In embodiments, administration of ketamine according to the disclosed methods will result in a reduction in at least one symptom of gaming disorder, for example as compared to a baseline determination made before such administration. In embodiments, the reduction in at least one symptom of gaming disorder may be a decrease of one or more points of a patient completed self-assessment, as described herein, and/or a decrease of one or more points in a clinical outcome assessment or index score, such as the IGD-20 and the IGDS9-SF. In embodiments, administration of ketamine according to the disclosed methods will result in an elimination of at least one symptom of gaming disorder, for example as compared to a baseline determination made before such administration. In embodiments, administration of ketamine according to the disclosed methods will result in an elimination of at least one symptom of gaming disorder, for example as compared to a baseline determination made before such administration.

In embodiments, administration of ketamine according to the disclosed methods will result in the patient having a reduction in at least one symptom of a gaming disorder, including impaired control over gaming behaviour, increasing priority given to gaming behaviour to the extent that gaming takes precedence over other life interests and daily activities, continuation or escalation of gaming behaviour despite negative consequences, preoccupation with gaming, withdrawal symptoms when gaming is taken away or not possible, tolerance, the need to spend more time gaming to satisfy the urge; inability to reduce playing, unsuccessful attempts to quit gaming; giving up other activities, loss of interest in previously enjoyed activities due to gaming; continuing to game despite problems, deceiving family members or others about the amount of time spent on gaming, the use of gaming to relieve negative moods, such as guilt or hopelessness; risk having jeopardised or lost a job or relationship due to gaming.

In embodiments, administration of ketamine according to the disclosed methods will result in the patient having an elimination of at least one symptom of a gaming disorder, including impaired control over gaming behaviour, increasing priority given to gaming behaviour to the extent that gaming takes precedence over other life interests and daily activities, continuation or escalation of gaming behaviour despite negative consequences, preoccupation with gaming, withdrawal symptoms when gaming is taken away or not possible, tolerance, the need to spend more time gaming to satisfy the urge; inability to reduce playing, unsuccessful attempts to quit gaming; giving up other activities, loss of interest in previously enjoyed activities due to gaming; continuing to game despite problems, deceiving family members or others about the amount of time spent on gaming, the use of gaming to relieve negative moods, such as guilt or hopelessness; risk having jeopardised or lost a job or relationship due to gaming.

In embodiments, the patient with gaming disorder has a comorbid psychiatric condition. In embodiments, treating the patient with gaming disorder according to the methods is also effective to treat one or more comorbid psychiatric conditions in the patient.

c. Compulsive Sexual Behaviour Disorder (CSBD)

In some aspects, the methods are useful in treating a patient with compulsive sexual behaviour disorder (CSBD). Whether a patient has CSBD can be determined according to ordinary skill.

For example, a patient may be determined to have CSBD based on the definition in the ICD-11, which defines compulsive sexual behavioural disorder as a persistent pattern of failure to control intense, repetitive sexual impulses or urges resulting in repetitive sexual behaviour. Exemplary criteria according to the ICD-11 for compulsive sexual behaviour disorder include: (1) a persistent pattern of failure to control intense, repetitive sexual impulses or urges resulting in repetitive sexual behaviour, manifested in one or more of the following: (a) engaging in repetitive sexual behaviour has become a central focus of the individual's life to the point of neglecting health and personal care or other interests, activities and responsibilities; (b) the individual has made numerous unsuccessful efforts to control or significantly reduce repetitive sexual behaviour, (c) the individual continues to engage in repetitive sexual behaviour despite adverse consequences (e.g., marital conflict due to sexual behaviour, financial or legal consequences, negative impact on health); and (d) the person continues to engage in repetitive sexual behaviour even when the individual derives little or no satisfaction from it. (2) the pattern of failure to control intense, repetitive sexual impulses or urges and resulting repetitive sexual behaviour is manifested over an extended period of time (e.g., 6 months or more); (3) the pattern of failure to control intense, repetitive sexual impulses or urges and resulting repetitive sexual behaviour is not better accounted for by another mental disorder (e.g., Manic Episode) or other medical condition and is not due to the effects of a substance or medication; (4) the pattern of repetitive sexual behaviour results in marked distress or significant impairment in personal, family, social, educational, occupational, or other important areas of functioning. Distress that is entirely related to moral judgments and disapproval about sexual impulses, urges, or behaviours is not sufficient to meet this requirement (ICD-11). Sexual addiction may also be referred to as compulsive sexual behaviour, hypersexuality, or hypersexuality disorder.

The presence of CSBD may be determined by the classification scale of the ICD-11, or based on ordinary skill. In embodiments, the Compulsive Sexual behaviour Inventory (CSBI-13), a 13-item, five point Likert scale, may be utilised to determine if a patient has compulsive sexual behaviour disorder. In embodiments, a score above a 35 indicates the presence of CSBD (Coleman, n.d.).

In embodiments, the term “compulsive sexual behaviour disorder patient” or “CSBD patient” (or “patient with compulsive sexual behaviour disorder” or “patient with CSBD”) refers to a patient diagnosed with CSBD, such as defined herein.

Herein, “reducing compulsive sexual behaviour” or “reduction in compulsive sexual behaviour” refers to reducing the amount or frequency of compulsive sexual behaviour, such as assessed by standardised screening tools like the CSBI-13. In embodiments, “reducing compulsive sexual behaviour” or “reduction in compulsive sexual behaviour” refers to a reduction in daily compulsive sexual behaviour, a reduction in the time spent engaging in compulsive sexual behaviour per day, or a reduction in the frequency of compulsive sexual behaviour, such as a reduction in the percentage of compulsive sexual behaviour days in general, or a reduction in the percentage of heavy compulsive sexual behaviour days. In embodiments, “reducing compulsive sexual behaviour” or “reduction in compulsive sexual behaviour” refers to an increase in the time to any compulsive sexual behaviour or time to the first heavy compulsive sexual behaviour day.

Herein, “compulsive sexual behaviour abstinence” or “in abstinence from compulsive sexual behaviour” refers to not engaging in compulsive sexual behaviour. In embodiments, “promoting compulsive sexual behaviour abstinence” or “promotion of compulsive sexual behaviour abstinence” refers to help maintaining abstinence from compulsive sexual behaviour, in particular after at least 1 day of not exhibiting compulsive sexual behaviour, for example, maintaining abstinence from compulsive sexual behaviour for a period of at least 1 day, at least 1 week, at least 1 month, or at least 1 year.

Herein, “compulsive sexual behaviour disorder patient in abstinence from compulsive sexual behaviour” refers to a patient with compulsive sexual behaviour disorder in abstinence from compulsive sexual behaviour for a period, for example, for at least 1 day, at least 1 week, at least 1 month, or at least 1 year.

Herein, “relapse into compulsive sexual behaviour” refers to engaging in compulsive sexual behaviour following a period of abstinence from compulsive sexual behaviour, for example following a period of compulsive sexual behaviour abstinence of at least 1 day, at least 1 week, at least 1 month, or at least 1 year.

Herein, “preventing relapse into compulsive sexual behaviour” refers to the prevention of engaging in compulsive sexual behaviour by a compulsive sexual behaviour disorder patient after the patient has stopped exhibiting compulsive sexual behaviour, in particular after 1 day or more of not exhibiting compulsive sexual behaviour. In embodiments, the term encompasses a delay in the resumption of compulsive sexual behaviour as compared to the time to resumption by a patient that is not administered a compound of the invention using the disclosed methods. The delay in resumption can be for at least 1 day, at least 1 week, at least 1 month, or at least 1 year.

In embodiments, the patient with CSBD is administered ketamine. In embodiments, the patient with CSBD receives KAP. In embodiments, the patient with CSBD undergoes a KAP treatment regimen. In embodiments, the administration of the ketamine is effective to treat CSBD. In embodiments, the KAP is effective to treat CSBD. In embodiments, the KAP treatment regimen is effective to treat CSBD. In embodiments, HNK or norketamine is administered. In embodiments, HNK or norketamine is effective to treat CSBD.

In embodiments, the administration of ketamine according to the disclosed methods will be effective to treat CSBD as shown by a reduction in the severity of CSBD. In embodiments, a patient who has CSBD characterised as being mild when measured at baseline will no longer have a mild CSBD. In embodiments, a patient who has CSBD characterised as being moderate when measured at baseline will no longer have a moderate CSBD. In embodiments, a patient who has CSBD characterised as being severe when measured at baseline will no longer have a severe CSBD A reduction in the severity of CSBD can be determined based on ordinary skill, such as using an assessment.

In embodiments, the administration of ketamine according to the disclosed methods will be effective to treat CSBD as shown by a reduction in CSBI score. In embodiments, a patient who scores 35 or higher on the Compulsive Sexual behaviour Inventory (CSBI-13) when measured at baseline will have a reduction in the CSBI-13 of at least 5 points.

In embodiments, the administration of ketamine according to the disclosed methods will be effective to treat CSBD as shown by one or more of: (a) a reduction in compulsive sexual behaviour, (b) a reduction in drive or urge to engage in compulsive sexual behaviour, (c) a promotion of abstinence from compulsive sexual behaviour, (d) a prevention of relapse into compulsive sexual behaviour, (e) a reduction in at least one symptom of CSBD.

In embodiments, administration of ketamine according to the disclosed methods will result in a measurable reduction in compulsive sexual behaviour or in the drive or urge to engage in compulsive sexual behaviour, for example as compared to a baseline determination made before such administration. In embodiments, the reduction in compulsive sexual behaviour is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from compulsive sexual behaviour or prevention of relapse into compulsive sexual behaviour, when measured from the start of treatment, such as the first administration. In embodiments, the promotion of abstinence from compulsive sexual behaviour results in abstinence for at least 1 day, at least 1 week, at least 1 month, or at least 1 year, from the start of treatment. In embodiments, the reduction in compulsive sexual behaviour, reduction in drive or urge to engage in compulsive sexual behaviour, promotion of abstinence from compulsive sexual behaviour, prevention of relapse into compulsive sexual behaviour, and reduction in at least one symptom of compulsive sexual behaviour disorder lasts for at least 1 day, at least 1 week, at least 1 month, or at least 1 year.

In embodiments, administration of ketamine according to the disclosed methods will result in a measurable improvement in at least one symptom of CSBD, for example as compared to a baseline determination made before such administration. In embodiments, the reduction in at least one symptom of CSBD may be a decrease of one or more points of a patient completed self-assessment, as described herein, and/or a decrease of one or more points in a clinical outcome assessment or index score, such as the CSBI-13. In embodiments, administration of ketamine according to the disclosed methods will result in an elimination of at least one symptom of compulsive sexual behaviour disorder, for example as compared to a baseline determination made before such administration.

In embodiments, administration of ketamine according to the disclosed methods will result in the patient having a reduction in at least one symptom of compulsive sexual behaviour disorder, including engaging in repetitive sexual behaviour has become a central focus of the individual's life to the point of neglecting health and personal care or other interests, activities and responsibilities; the individual has made numerous unsuccessful efforts to control or significantly reduce repetitive sexual behaviour, the individual continues to engage in repetitive sexual behaviour despite adverse consequences; and the person continues to engage in repetitive sexual behaviour even when the individual derives little or no satisfaction from it.

In embodiments, administration of ketamine according to the disclosed methods will result in the patient having an elimination of at least one symptom of compulsive sexual behaviour disorder, including engaging in repetitive sexual behaviour has become a central focus of the individual's life to the point of neglecting health and personal care or other interests, activities and responsibilities; the individual has made numerous unsuccessful efforts to control or significantly reduce repetitive sexual behaviour, the individual continues to engage in repetitive sexual behaviour despite adverse consequences; and the person continues to engage in repetitive sexual behaviour even when the individual derives little or no satisfaction from it.

In embodiments, the patient with CSBD has a comorbid psychiatric condition. In embodiments, treating the patient with CSBD according to the disclosed methods is also effective to treat one or more comorbid psychiatric conditions in the patient.

d. Compulsive Buying-Shopping Disorder

In some aspects, the methods are useful in treating a patient with compulsive buying-shopping disorder. In embodiments, “compulsive buying-shopping disorder” includes related compulsive buying or compulsive shopping disorders, such as compulsive buying behaviour disorder, compulsive buying disorder (CBD), compulsive buying behaviour (CBB), compulsive online shopping disorder, compulsive spending disorder, spending addiction, and “oniomania.” Whether a patient has compulsive buying-shopping disorder can be determined according to ordinary skill.

For example, a patient may be determined to have compulsive buying-shopping disorder based on the definition provided by Black, 2001. Black, 2001 defines “compulsive buying disorder” as excessive or poorly controlled preoccupations, urges or behaviours regarding shopping and spending, which lead to adverse consequences (Black, CNS Drugs, 2001; 15(1):17-27; Granero, Front Psychol., 2016; 7:914). Exemplary diagnostic criteria include (a) feeling overly preoccupied with shopping and spending, (b) feeling that one's shopping behaviour is excessive, inappropriate or uncontrolled, and (c) one's shopping desires, urges, fantasies or behaviours being overly time consuming, causing them to feel upset or guilty, or leading to serious problems in their life including financial/legal problems and relationship loss (Black, 2001).

The ICD-11 categorizes compulsive buying-shopping disorder as an unspecified mental, behavioural, or neurodevelopmental disorder. Diagnostic criteria include (a) a repeated failure to resist a strong impulse, drive, or urge to perform an act that is rewarding to the person, at least in the short-term, despite longer-term harm either to the individual or to others; (b) marked distress about the behaviour pattern; and/or (c) significant impairment in personal, family, social, educational, occupational, or other important areas of functioning.

Compulsive buying-shopping disorder may be characterised as being mild, moderate, and severe. The severity of compulsive buying-shopping disorder may be determined via the modified Yale Brown Obsessive-Compulsive Scale, the YBOCS-Shopping Scale (YBOCS-SV), or based on ordinary skill. In embodiments, a patient may have compulsive buying-shopping disorder characterised as being mild. In other embodiments, a patient may have compulsive buying-shopping disorder characterised as being moderate. In other embodiments, a patient may have compulsive buying-shopping disorder characterised as being severe.

In embodiments, the term “compulsive buying-shopping disorder patient” (or “patient with compulsive buying-shopping disorder”) refers to a patient diagnosed with compulsive buying-shopping disorder, such as defined herein.

Herein, “reducing compulsive buying” or “reduction in compulsive buying” refers to reducing the amount or frequency of compulsive buying, such as assessed by standardised screening tools like the YBOCS-SV. In embodiments, “reducing compulsive buying” or “reduction in compulsive buying” refers to a reduction in daily compulsive buying, a reduction in the time spent compulsive buying per day, or a reduction in the frequency of compulsive buying, such as a reduction in the percentage of compulsive buying days in general, or a reduction in the percentage of heavy compulsive buying days. In embodiments, “reducing compulsive buying” or “reduction in compulsive buying” refers to an increase in the time to any compulsive buying or time to the first heavy compulsive buying day.

Herein, “compulsive buying abstinence” or “in abstinence from compulsive buying” refers to not engaging in compulsive buying. In embodiments, “promoting compulsive buying abstinence” or “promotion of compulsive buying abstinence” refers to help maintaining abstinence from compulsive buying, in particular after at least 1 day of not engaging in compulsive buying, for example, maintaining abstinence from compulsive buying for a period of at least 1 day, at least 1 week, at least 1 month, or at least 1 year.

Herein, “compulsive buying-shopping disorder patient in abstinence from compulsive buying” refers to a patient with compulsive buying-shopping disorder in abstinence from compulsive buying for a period, for example, for at least 1 day, at least 1 week, at least 1 month, or at least 1 year.

Herein, “relapse into compulsive buying” refers to compulsive buying following a period of abstinence from compulsive buying, for example following a period of compulsive buying abstinence of at least 1 day, at least 1 week, at least 1 month, or at least 1 year.

Herein, “preventing relapse into compulsive buying” refers to the prevention of compulsive buying by a compulsive buying-shopping disorder patient after the patient has stopped compulsive buying, in particular after 1 day or more of not engaging in compulsive buying. In embodiments, the term encompasses a delay in the resumption of compulsive buying as compared to the time to resumption by a patient that is not administered a compound of the invention using the disclosed methods. The delay in resumption can be for at least 1 day, at least 1 week, at least 1 month, or at least 1 year.

In embodiments, the patient with compulsive buying-shopping disorder is administered ketamine. In embodiments, the patient with compulsive buying-shopping disorder receives KAP. In embodiments, the patient with compulsive buying-shopping disorder undergoes a KAP treatment regimen. In embodiments, the administration of the ketamine is effective to treat compulsive buying-shopping disorder. In embodiments, the KAP is effective to treat compulsive buying-shopping disorder. In embodiments, the KAP treatment regimen is effective to treat compulsive buying-shopping disorder. In embodiments, HNK or norketamine is administered. In embodiments, HNK or norketamine is effective to treat compulsive buying-shopping disorder.

In embodiments, the administration of ketamine according to the disclosed methods will be effective to treat compulsive buying-shopping disorder as shown by a reduction in the severity of compulsive buying-shopping disorder. In embodiments, a patient who has compulsive buying-shopping disorder characterised as being mild when measured at baseline will no longer have a mild compulsive buying-shopping disorder. In embodiments, a patient who has compulsive buying-shopping disorder characterised as being moderate when measured at baseline will no longer have a moderate compulsive buying-shopping disorder. A reduction in the severity of compulsive buying-shopping disorder can be determined based on ordinary skill, such as using an assessment. For example, a patient who has compulsive buying-shopping disorder characterised as being severe when measured at baseline will no longer have a severe compulsive buying-shopping disorder. In embodiments, a patient who scores 20 or higher on the YBOCS-SV when measured at baseline will have a reduction in the YBOCS-SV of at least 3. In embodiments, a patient who scores 30 or higher on the YBOCS-SV when measured at baseline will have a reduction in the YBOCS-SV of at least 5.

In embodiments, the administration of ketamine according to the disclosed methods will be effective to treat compulsive buying-shopping disorder as shown by one or more of: (a) a reduction in compulsive buying, (b) a reduction in drive or urge to engage in compulsive buying, (c) a promotion of abstinence from compulsive buying, (d) a prevention of relapse into compulsive buying, (e) a reduction in at least one symptom of compulsive buying-shopping disorder.

In embodiments, administration of ketamine according to the disclosed methods will result in a measurable reduction in compulsive buying or in the drive or urge to engage in compulsive buying, for example as compared to a baseline determination made before such administration. In embodiments, the reduction in compulsive buying is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from compulsive buying or prevention of relapse into compulsive buying, when measured from the start of treatment, such as the first administration. In embodiments, the promotion of abstinence from compulsive buying results in abstinence for at least 1 day, at least 1 week, at least 1 month, or at least 1 year, from the start of treatment. In embodiments, the reduction in compulsive buying, reduction in drive or urge to engage in compulsive buying, promotion of abstinence from compulsive buying, prevention of relapse into compulsive buying, and reduction in at least one symptom of compulsive buying-shopping disorder lasts for at least 1 day, at least 1 week, at least 1 month, or at least 1 year.

In embodiments, administration of ketamine according to the disclosed methods will result in a measurable improvement in at least one symptom of compulsive buying-shopping disorder, for example as compared to a baseline determination made before such administration. In embodiments, the reduction in at least one symptom of compulsive buying-shopping disorder may be a decrease of one or more points of a patient completed self-assessment, as described herein, and/or a decrease of one or more points in a clinical outcome assessment or index score, such as the YBOCS-SV. In embodiments, administration of ketamine according to the disclosed methods will result in an elimination of at least one symptom of compulsive buying-shopping disorder, for example as compared to a baseline determination made before such administration.

In embodiments, administration of ketamine according to the disclosed methods will result in the patient having a reduction in at least one symptom of compulsive buying-shopping disorder, including a repeated failure to resist a strong impulse, drive, or urge to perform an act that is rewarding to the person, at least in the short-term, despite longer-term harm either to the individual or to others; marked distress about the behaviour pattern, significant impairment in personal, family, social, educational, occupational, or other important areas of functioning; feeling overly preoccupied with shopping and spending, feeling that one's shopping behaviour is excessive, inappropriate or uncontrolled; one's shopping desires, urges, fantasies or behaviours being overly time consuming, causing them to feel upset or guilty, or leading to serious problems in their life including financial/legal problems and relationship loss.

In embodiments, administration of ketamine according to the disclosed methods will result in the patient having an elimination of at least one symptom of compulsive buying-shopping disorder, including a repeated failure to resist a strong impulse, drive, or urge to perform an act that is rewarding to the person, at least in the short-term, despite longer-term harm either to the individual or to others; marked distress about the behaviour pattern, significant impairment in personal, family, social, educational, occupational, or other important areas of functioning; feeling overly preoccupied with shopping and spending, feeling that one's shopping behaviour is excessive, inappropriate or uncontrolled; one's shopping desires, urges, fantasies or behaviours being overly time consuming, causing them to feel upset or guilty, or leading to serious problems in their life including financial/legal problems and relationship loss.

In embodiments, the patient with compulsive buying-shopping disorder has a comorbid psychiatric condition. In embodiments, treating the patient with compulsive buying-shopping disorder is also effective to treat one or more comorbid psychiatric conditions in the patient.

e. Technology Addiction

In some aspects, the methods are useful in treating a patient with technology addiction. Technology addiction includes compulsive technology use (CTU), as well as specific types including internet addiction, smartphone addiction, compulsive mobile app use, mobile email addiction, and information addiction.

Whether a patient has technology addiction can be determined according to ordinary skill. Exemplary criteria include those disclosed by Sternlicht and Sternlicht, 2022, which include: (a) inability to moderate or abstain from technology or a specific digital medium, (b) preoccupation with thinking about using technological devices, (c) compulsive technological use or experiencing cravings and urges to use digital devices, (d) neglecting important life areas such as work, school or relationships at the expense of technology, (e) continuing to use digital devices despite it contributing to consequences in your life, (f) losing interest in social and leisure activities that you once enjoyed at the expense of technology, (g) using digital devices in dangerous situations such as while driving a car or walking across a city street, (h) experiencing unwanted mental health symptoms such as depression, anxiety, stress or irritability at the expense of technological usage, (i) using digital devices to induce pleasure or experience gratification, (j) lying or hiding digital usage from family, friends or colleagues as a result of guilt or shame; and (k) using digital devices for longer durations than intended or finding yourself using digital devices with increased frequency over time.

Evidence shows that plasma dopamine levels are positively correlated with aspects of internet addiction (Liu & Luo, Int J Clin Exp Med. 2015; 8(6): 9943-9948). Internet activities, for example, winning a level of a video game or getting “likes” on a picture may enhance dopamine release.

Technology addiction may be characterised as being mild, moderate, and severe. The severity of technology addiction may be determined by the Digital Addiction Scale (DAS), wherein addiction is rated on a scale of 1-5, 5 being the highest addiction level (Kesici & Tunc, 2018), or based on ordinary skill. In embodiments, a patient may have technology addiction characterised as being mild. In other embodiments, a patient may have technology addiction characterised as being moderate. In other embodiments, a patient may have technology addiction characterised as being severe.

In embodiments, the term “technology addiction patient” refers to a patient diagnosed with technology addiction, such as defined herein.

Herein, “reducing compulsive technology use” or “reduction in compulsive technology use” refers to reducing the amount or frequency of compulsive technology use, such as assessed by standardised screening tools like the DAS. In embodiments, “reducing compulsive technology use” or “reduction in compulsive technology use” refers to a reduction in daily compulsive technology use, a reduction in the time spent compulsively using technology per day, or a reduction in the frequency of compulsive technology use, such as a reduction in the percentage of compulsive technology use days in general, or a reduction in the percentage of heavy compulsive technology use days. In embodiments, “reducing compulsive technology use” or “reduction in compulsive technology use” refers to an increase in the time to any compulsive technology use or time to the first heavy compulsive technology use day.

Herein, “technology addiction abstinence” or “in abstinence from technology” refers to not engaging in compulsive technology use. In embodiments, “promoting compulsive technology use abstinence” or “promotion of compulsive technology use abstinence” refers to help maintaining abstinence from compulsive technology use, in particular after at least 1 day of not engaging in compulsive technology use, for example, maintaining abstinence from compulsive technology use for a period of at least 1 day, at least 1 week, at least 1 month, or at least 1 year.

Herein, “technology addiction patient in abstinence from compulsive technology use” refers to a patient with technology addiction in abstinence from compulsive technology use for a period, e.g., for at least 1 day, at least 1 week, at least 1 month, or at least 1 year.

Herein, “relapse into compulsive technology use” refers to engaging in compulsive technology use following a period of abstinence from compulsive technology use, for example following a period of compulsive technology use abstinence of at least 1 day, at least 1 week, at least 1 month, or at least 1 year.

Herein, “preventing relapse into compulsive technology use” refers to the prevention of engaging in compulsive technology use by a technology addiction patient after the patient has stopped exhibiting compulsive technology use, in particular after 1 day or more of not exhibiting compulsive technology use. In embodiments, the term encompasses a delay in the resumption of compulsive technology use as compared to the time to resumption by a patient that is not administered a compound of the invention using the disclosed methods. The delay in resumption can be for at least 1 day, at least 1 week, at least 1 month, or at least 1 year.

In embodiments, the patient with technology addiction is administered ketamine. In embodiments, the patient with technology addiction receives KAP. In embodiments, the patient with technology addiction undergoes a KAP treatment regimen. In embodiments, the administration of the ketamine is effective to treat technology addiction. In embodiments, the KAP is effective to treat technology addiction. In embodiments, the KAP treatment regimen is effective to treat technology addiction. In embodiments, HNK or norketamine is administered. In embodiments HNK or norketamine is effective to treat technology addiction.

In embodiments, the administration of ketamine according to the disclosed methods will be effective to treat technology addiction as shown by a reduction in the severity of technology addiction. In embodiments, a patient who has technology addiction characterised as being mild when measured at baseline will no longer have a mild technology addiction. In embodiments, a patient who has technology addiction characterised as being moderate when measured at baseline will no longer have a moderate technology addiction. In embodiments, a patient who has technology addiction characterised as being severe when measured at baseline will no longer have a severe technology addiction. A reduction in the severity of technology addiction can be determined based on ordinary skill, such as using an assessment. For example, in embodiments, a patient who has a DAS score of 2 or higher when measured at baseline will have a reduction in DAS score of at least 1 point. In embodiments, a patient who has a DAS score of 3 or higher when measured at baseline will have a reduction in DAS score of at least 2 points. In embodiments, a patient who has a DAS score of 5 when measured at baseline will have a reduction in DAS score of at least 3 points.

In embodiments, the administration of ketamine according to the disclosed methods will be effective to treat technology addiction as shown by one or more of: (a) a reduction in compulsive technology use, (b) a reduction in drive or urge to engage in compulsive technology use, (c) a promotion of abstinence from compulsive technology use, (d) a prevention of relapse into compulsive technology use, (e) a reduction in at least one symptom of technology addiction.

In embodiments, administration of ketamine according to the disclosed methods will result in a measurable reduction in compulsive technology use or in the drive or urge to engage in compulsive technology use, for example as compared to a baseline determination made before such administration. In embodiments, the reduction in compulsive technology use is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from compulsive technology use or prevention of relapse into compulsive technology use, when measured from the start of treatment, such as the first administration. In embodiments, the promotion of abstinence from compulsive technology use results in abstinence for at least 1 day, at least 1 week, at least 1 month, or at least 1 year, from the start of treatment. In embodiments, the reduction in compulsive technology use, reduction in drive or urge to engage in compulsive technology use, promotion of abstinence from compulsive technology use, prevention of relapse into compulsive technology use, and reduction in at least one symptom of technology addiction lasts for at least 1 day, at least 1 week, at least 1 month, or at least 1 year.

In embodiments, administration of ketamine according to the disclosed methods will result in a measurable improvement in at least one symptom of technology addiction, for example as compared to a baseline determination made before such administration. In embodiments, the reduction in at least one symptom of technology addiction may be a decrease of one or more points of a patient completed self-assessment, as described herein, and/or a decrease of one or more points in a clinical outcome assessment or index score, such as the DAS. In embodiments, administration of ketamine according to the disclosed methods will result in an elimination of at least one symptom of technology addiction, for example as compared to a baseline determination made before such administration.

In embodiments, administration of ketamine according to the disclosed methods will result in the patient having a reduction in at least one symptom of technology addiction, including inability to moderate or abstain from technology or a specific digital medium, preoccupation with thinking about using technological devices, compulsive technological use or experiencing cravings and urges to use digital devices, neglecting important life areas such as work, school or relationships at the expense of technology, continuing to use digital devices despite it contributing to consequences in your life, losing interest in social and leisure activities that you once enjoyed at the expense of technology, using digital devices in dangerous situations such as while driving a car or walking across a city street, experiencing unwanted mental health symptoms such as depression, anxiety, stress or irritability at the expense of technological usage, using digital devices to induce pleasure or experience gratification, lying or hiding digital usage from family, friends or colleagues as a result of guilt or shame; and using digital devices for longer durations than intended or finding yourself using digital devices with increased frequency over time.

In embodiments, administration of ketamine according to the disclosed methods will result in the patient having an elimination of at least one symptom of technology addiction, including inability to moderate or abstain from technology or a specific digital medium, preoccupation with thinking about using technological devices, compulsive technological use or experiencing cravings and urges to use digital devices, neglecting important life areas such as work, school or relationships at the expense of technology, continuing to use digital devices despite it contributing to consequences in your life, losing interest in social and leisure activities that you once enjoyed at the expense of technology, using digital devices in dangerous situations such as while driving a car or walking across a city street, experiencing unwanted mental health symptoms such as depression, anxiety, stress or irritability at the expense of technological usage, using digital devices to induce pleasure or experience gratification, lying or hiding digital usage from family, friends or colleagues as a result of guilt or shame; and using digital devices for longer durations than intended or finding yourself using digital devices with increased frequency over time.

In embodiments, the patient with technology addiction has a comorbid psychiatric condition. In embodiments, treating the patient with technology addiction is also effective to treat one or more comorbid psychiatric conditions in the patient.

f. Kleptomania

In some aspects, the methods are useful in treating a patient with kleptomania. Whether a patient has kleptomania can be determined according to ordinary skill.

For example, a patient may be determined to have kleptomania based on the definition in the DSM-V. The DSM-V defines kleptomania as a disorder characterised by the recurrent failure to resist impulses to steal items even though the items are not required for personal use or are of little value to the individual. The prevalence of kleptomania in the general population is rare, roughly between 0.3% to about 0.6%, and is higher in females than in males by about 3:1 (APA, 2013). Kleptomania is estimated to occur in about 4% to about 24% of individuals arrested for shoplifting (APA, 2013).

DSM-V diagnostic criteria for kleptomania include (a) recurrent failure to resist impulses to steal objects that are not needed for personal use or for their monetary value, (b) increasing sense of tension immediately before committing the theft, and (c) pleasure, gratification, or relief at the time of committing the theft. Additionally, the stealing not being committed to express anger or vengeance and not in response to a delusion or a hallucination, and the stealing not being better explained by conduct disorder, a manic episode, or antisocial personality disorder (APA, 2013).

The ICD-11 defines kleptomania as a recurrent failure to control strong impulses to steal objects in the absence of an apparent motive (e.g., objects are not acquired for personal use or monetary gain). ICD-11 diagnostic criteria for kleptomania include (a) a recurrent failure to control strong impulses to steal objects, (b) lack of an apparent motive for stealing objects (e.g., objects are not acquired for personal use or monetary gain), (c) the individual experiences increased tension or affective arousal prior to instances of theft or attempted theft, (d) the individual experiences pleasure, excitement, relief, or gratification during and immediately following the act of stealing, and (e) the acts of theft or attempted theft are not better accounted for by a disorder of intellectual development, another mental disorder (e.g., a manic episode), or substance intoxication (WHO, 2022).

Kleptomania may be characterised as being mild, moderate, and severe. The severity of kleptomania may be determined by the Kleptomania Symptom Assessment Scale (K-SAS), wherein scores ranging from 31 to 44 reflect severe kleptomania symptoms, scores ranging from 21 to 30 indicate moderate symptoms, scores ranging from 8-20 indicate mild symptoms, and scores below 8 indicate remission (Hollander and Berlin, 2008); or based on ordinary skill. In embodiments, a patient may have kleptomania characterised as being mild. In other embodiments, a patient may have kleptomania characterised as being moderate. In other embodiments, a patient may have kleptomania characterised as being severe. In embodiments, the Clinical Global Impressions scale (both severity and improvement measures), Sheehan Disability Scale (SDS), and Global Assessment of Functioning (GAF) may additionally be utilised.

In embodiments, “kleptomania disorder patient” (or “patient with kleptomania disorder”) refers to a patient diagnosed with kleptomania, such as defined herein.

Herein, “reducing stealing” or “reduction in stealing” refers to reducing the amount or frequency of stealing, such as assessed by standardised screening tools like K-SAS. In embodiments, “reducing stealing” or “reduction in stealing” refers to a reduction in daily stealing, a reduction in time spent stealing per day, or a reduction in the frequency of stealing, such as a reduction in the percentage of stealing days in general, or a reduction in the percentage of heavy stealing days. In embodiments, “reducing stealing” or “reduction in stealing” refers to an increase in time to any stealing or time to the first heavy stealing day.

Herein, “kleptomania abstinence” or “in abstinence from stealing” refers to not stealing. In embodiments, “promoting kleptomania abstinence” or “promotion of kleptomania abstinence” refers to help maintaining abstinence from stealing, in particular after at least 1 day of not stealing, for example, maintaining abstinence from stealing for a period of at least 1 day, at least 1 week, at least 1 month, or at least 1 year.

Herein, “kleptomania patient in abstinence from stealing” refers to a patient with kleptomania in abstinence from stealing for a period, for example, for at least 1 day, at least 1 week, at least 1 month, or at least 1 year.

Herein, “relapse into stealing” refers to stealing following a period of abstinence from stealing, for example following a period of stealing abstinence of at least 1 day, at least 1 week, at least 1 month, or at least 1 year.

Herein, “preventing relapse into stealing” refers to the prevention of stealing by a kleptomania patient after the patient has stopped stealing, in particular after 1 day or more of not stealing. In embodiments, the term encompasses a delay in the resumption of stealing as compared to the time to resumption by a patient that is not administered a compound of the invention using the disclosed methods. The delay in resumption can be for at least 1 day, at least 1 week, at least 1 month, or at least 1 year.

In embodiments, the patient with kleptomania is administered ketamine. In embodiments, the patient with kleptomania receives KAP. In embodiments, the patient with kleptomania undergoes a KAP treatment regimen. In embodiments, the administration of the ketamine is effective to treat kleptomania. In embodiments, the KAP is effective to treat kleptomania. In embodiments, the KAP treatment regimen is effective to treat kleptomania. In embodiments, HNK or norketamine is administered. In embodiments, HNK or norketamine is effective to treat kleptomania.

In embodiments, the administration of ketamine according to the disclosed methods will be effective to treat kleptomania as shown by a reduction in the severity of kleptomania. In embodiments, a patient who has kleptomania characterised as being mild when measured at baseline will no longer have mild kleptomania. In embodiments, a patient who has kleptomania characterised as being moderate when measured at baseline will no longer have moderate kleptomania. In embodiments, a patient who has kleptomania characterised as being severe when measured at baseline will no longer have severe kleptomania. A reduction in the severity of kleptomania can be determined based on ordinary skill, such as using an assessment. For example, in embodiments, a patient who scores at least 21 on the K-SAS when measured at baseline will have a reduction in the K-SAS score of at least 4 points. In embodiments, a patient who scores 31 or higher on the K-SAS when measured at baseline will have a reduction in the K-SAS score of at least 6 points.

In embodiments, the administration of ketamine according to the disclosed methods will be effective to treat kleptomania as shown by one or more of: (a) a reduction in stealing, (b) a reduction in drive or urge to engage in stealing, (c) a promotion of abstinence from stealing, (d) a prevention of relapse into stealing, (e) a reduction in at least one symptom of kleptomania.

In embodiments, administration of ketamine according to the disclosed methods will result in a measurable reduction in stealing or in the drive or urge to engage in stealing, for example as compared to a baseline determination made before such administration. In embodiments, the reduction in stealing is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from stealing or prevention of relapse into stealing, when measured from the start of treatment, such as the first administration. In embodiments, the promotion of abstinence from stealing results in abstinence for at least 1 day, at least 1 week, at least 1 month, or at least 1 year, from the start of treatment. In embodiments, the reduction in stealing, reduction in drive or urge to engage in stealing, promotion of abstinence from stealing, prevention of relapse into stealing, and reduction in at least one symptom of kleptomania lasts for at least 1 day, at least 1 week, at least 1 month, or at least 1 year.

In embodiments, administration of ketamine according to the disclosed methods will result in a measurable improvement in at least one symptom of kleptomania, for example as compared to a baseline determination made before such administration. In embodiments, the reduction in at least one symptom of kleptomania may be a decrease of one or more points of a patient completed self-assessment, as described herein, and/or a decrease of one or more points in a clinical outcome assessment or index score, such as the K-SAS. In embodiments, administration of ketamine according to the disclosed methods will result in an elimination of at least one symptom of kleptomania, for example as compared to a baseline determination made before such administration.

In embodiments, administration of ketamine according to the disclosed methods will result in the patient having a reduction in at least one symptom of kleptomania, including recurrent failure to resist impulses to steal objects that are not needed for personal use or for their monetary value, increasing sense of tension immediately before committing the theft; pleasure, gratification, or relief at the time of committing the theft; a recurrent failure to control strong impulses to steal objects, lack of an apparent motive for stealing objects, the individual experiences increased tension or affective arousal prior to instances of theft or attempted theft, and the individual experiences pleasure, excitement, relief, or gratification during and immediately following the act of stealing.

In embodiments, administration of ketamine according to the disclosed methods will result in the patient having an elimination of at least one symptom of kleptomania, including recurrent failure to resist impulses to steal objects that are not needed for personal use or for their monetary value, increasing sense of tension immediately before committing the theft; pleasure, gratification, or relief at the time of committing the theft; a recurrent failure to control strong impulses to steal objects, lack of an apparent motive for stealing objects, the individual experiences increased tension or affective arousal prior to instances of theft or attempted theft, and the individual experiences pleasure, excitement, relief, or gratification during and immediately following the act of stealing.

In embodiments, the patient with kleptomania has a comorbid psychiatric condition. In embodiments, treating the patient with kleptomania is also effective to treat one or more comorbid psychiatric conditions in the patient.

g. Pyromania

In some aspects, the methods are useful in treating a patient with pyromania. Whether a patient has pyromania can be determined according to ordinary skill.

For example, a patient may be determined to have pyromania based on the definition in the DSM-V. The DSM-V defines pyromania as a disorder characterised by multiple episodes of deliberate and purposeful fire setting associated with tension or affective arousal.

DSM-V diagnostic criteria for pyromania include (a) deliberate and purposeful fire setting on more than 1 occasion, (b) tension or affective arousal before the act, (c) fascination with, interest in, curiosity about, or attraction to fire and its situational contexts (e.g. —paraphernalia, uses, consequences); and (d) pleasure, gratification, or relief when setting fires or when witnessing or participating in their aftermath. Additionally, the fire setting is not done for monetary gain, as an expression of sociopolitical ideology, to conceal criminal activity, to express anger or vengeance, to improve one's living circumstances, in response to a delusion or hallucination, or as a result of impaired judgment (e.g., major neurocognitive disorder, intellectual disability, substance intoxication); and the fire setting is not better explained by conduct disorder, a manic episode, or antisocial personality disorder.

The ICD-11 defines pyromania as a recurrent failure to control strong impulses to set fires, resulting in multiple acts of, or attempts at, setting fire to property or other objects, in the absence of an apparent motive (e.g., monetary gain, revenge, sabotage, political statement, attracting attention or recognition) (WHO, 2022).

ICD-11 diagnostic criteria for pyromania include (a) a recurrent failure to control strong impulses to set fires, resulting in multiple acts of, or attempts at, setting fire to property or other objects, (b) lack of an apparent motive for the acts of, or attempts at, fire setting (e.g., monetary gain, revenge, sabotage, political statement, attracting recognition), (c) persistent fascination or preoccupation with fire and related stimuli (e.g., watching fires, building fires, fascination with firefighting equipment), (d) the individual experiences increased tension or affective arousal prior to instances of, or attempts at, fire setting, (e) the individual experiences pleasure, excitement, relief or gratification during, and immediately following the act of setting the fire, witnessing its effects, or participating in its aftermath, (f) acts of, or attempts at, fire setting are not better accounted for by a disorder of intellectual development, another mental disorder (e.g., a manic episode), or substance intoxication.

In embodiments, the fire setting scale, fire proclivity scale, and St. Andrews Fire and Risk Instrument (SAFARI) may be utilised to distinguish firesetters with non-firesetters. The fire setting scale and fire proclivity scale are each discussed in Gannon and Barrowcliffe, 2010, which is herein incorporated by reference, while SAFARI is discussed in Long et al., 2013, which is also herein incorporated by reference.

In embodiments, the term “pyromania disorder patient” (or “patient with pyromania disorder”) refers to a patient diagnosed with pyromania, such as defined herein.

Herein, “reducing starting fires” or “reduction in fire starting” refers to reducing the amount or frequency of fire starting, such as assessed by standardised screening tools like the fire proclivity scale, the fire setting scale, and SAFARI. In embodiments, “reducing fire starting” or “reduction in fire starting” refers to a reduction in daily fire starting, a reduction in the time spent starting fires per day, or a reduction in the frequency of fire starting, such as a reduction in the percentage of fire starting days in general, or a reduction in the percentage of heavy fire starting days. In embodiments, “reducing fire starting” or “reduction in fire starting” refers to an increase in the time to any fire starting or time to the first heavy fire starting day.

Herein, “pyromania abstinence” or “in abstinence from fire starting” refers to not engaging in fire starting. In embodiments, “promoting pyromania abstinence” or “promotion of pyromania abstinence” refers to help maintaining abstinence from fire starting, in particular after at least 1 day of not starting fires, for example, maintaining abstinence from fire starting for a period of at least 1 day, at least 1 week, at least 1 month, or at least 1 year.

Herein, “pyromania patient in abstinence from fire starting” refers to a patient with pyromania in abstinence from fire starting for a period, for example, for at least 1 day, at least 1 week, at least 1 month, or at least 1 year.

Herein, “relapse into fire starting” refers to starting fires following a period of abstinence from starting fires, for example following a period of fire starting abstinence of at least 1 day, at least 1 week, at least 1 month, or at least 1 year.

Herein, “preventing relapse into fire starting” refers to the prevention of fire starting by a pyromania patient after the patient has stopped starting fires, in particular after 1 day or more of not starting fires. In embodiments, the term encompasses the permanent stoppage of fire starting. In embodiments, the term encompasses a delay in the resumption of fire starting as compared to the time to resumption by a patient that is not administered a compound of the invention using the disclosed methods. The delay in resumption can be for at least 1 day, at least 1 week, at least 1 month, or at least 1 year.

In embodiments, the patient with pyromania is administered ketamine. In embodiments, the patient with pyromania receives KAP. In embodiments, the patient with pyromania undergoes a KAP treatment regimen. In embodiments, the administration of the ketamine is effective to treat pyromania. In embodiments, the KAP is effective to treat pyromania. In embodiments, the KAP treatment regimen is effective to treat pyromania. In embodiments, HNK or norketamine is administered. In embodiments, HNK or norketamine is effective to treat pyromania.

In embodiments, the administration of ketamine according to the disclosed methods will be effective to treat pyromania as shown by a reduction in the severity of pyromania. In embodiments, a patient who has pyromania characterised as being mild when measured at baseline will no longer have a mild pyromania. In embodiments, a patient who has pyromania characterised as being moderate when measured at baseline will no longer have a moderate pyromania. In embodiments, a patient who has pyromania characterised as being severe when measured at baseline will no longer have a severe pyromania A reduction in the severity of pyromania can be determined based on ordinary skill, such as using an assessment.

In embodiments, a patient who was initially classed as a firesetter may, after treatment, be classed as a non-firesetter.

In embodiments, the administration of ketamine according to the disclosed methods will be effective to treat pyromania as shown by one or more of: (a) a reduction in fire starting, (b) a reduction in drive or urge to engage in fire starting, (c) a promotion of abstinence from fire starting, (d) a prevention of relapse into fire starting, (e) a reduction in at least one symptom of pyromania.

In embodiments, administration of ketamine according to the disclosed methods will result in a measurable reduction in starting fires or in the drive or urge to engage in fire starting, for example as compared to a baseline determination made before such administration. In embodiments, the reduction in starting fires is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from starting fires or prevention of relapse into starting fires, when measured from the start of treatment, such as the first administration. In embodiments, the promotion of abstinence from starting fires results in abstinence for at least 1 day, at least 1 week, at least 1 month, or at least 1 year, from the start of treatment. In embodiments, the reduction in fire starting, reduction in drive or urge to engage in fire starting, promotion of abstinence from fire starting, prevention of relapse into fire starting, and reduction in at least one symptom of pyromania lasts for at least 1 day, at least 1 week, at least 1 month, or at least 1 year.

In embodiments, administration of ketamine according to the disclosed methods will result in a measurable improvement in at least one symptom of pyromania, for example as compared to a baseline determination made before such administration. In embodiments, the reduction in at least one symptom of pyromania may be a decrease of one or more points of a patient completed self-assessment, as described herein, and/or a decrease of one or more points in a clinical outcome assessment or index score, such as any of the fire setting scale, fire proclivity scale, and St. Andrews Fire and Risk Instrument (SAFARI). In embodiments, administration of ketamine according to the disclosed methods will result in an elimination of at least one symptom of pyromania, for example as compared to a baseline determination made before such administration.

In embodiments, administration of ketamine according to the disclosed methods will result in the patient having a reduction in at least one symptom of pyromania, including deliberate and purposeful fire setting on more than 1 occasion, tension or affective arousal before the act, fascination with, interest in, curiosity about, or attraction to fire and its situational contexts; pleasure, gratification, or relief when setting fires or when witnessing or participating in their aftermath; a recurrent failure to control strong impulses to set fires, resulting in multiple acts of, or attempts at, setting fire to property or other objects; lack of an apparent motive for the acts of, or attempts at, fire setting; persistent fascination or preoccupation with fire and related stimuli, the individual experiences increased tension or affective arousal prior to instances of, or attempts at, fire setting; the individual experiences pleasure, excitement, relief or gratification during, and immediately following the act of setting the fire, witnessing its effects, or participating in its aftermath.

In embodiments, administration of ketamine according to the disclosed methods will result in the patient having an elimination of at least one symptom of pyromania, including deliberate and purposeful fire setting on more than 1 occasion, tension or affective arousal before the act, fascination with, interest in, curiosity about, or attraction to fire and its situational contexts; pleasure, gratification, or relief when setting fires or when witnessing or participating in their aftermath; a recurrent failure to control strong impulses to set fires, resulting in multiple acts of, or attempts at, setting fire to property or other objects; lack of an apparent motive for the acts of, or attempts at, fire setting; persistent fascination or preoccupation with fire and related stimuli, the individual experiences increased tension or affective arousal prior to instances of, or attempts at, fire setting; the individual experiences pleasure, excitement, relief or gratification during, and immediately following the act of setting the fire, witnessing its effects, or participating in its aftermath.

In embodiments, the patient with pyromania has a comorbid psychiatric condition. In embodiments, treating the patient with pyromania is also effective to treat one or more comorbid psychiatric conditions in the patient.

h. Internet Addiction

In some aspects, the methods are useful in treating a patient with internet addiction. Internet addiction also may include related conditions such as the compulsive use of electronic devices, compulsive texting, and compulsively checking social media. Whether a patient has internet addiction can be determined according to ordinary skill.

For example, a patient may be determined to have internet addiction based on the definition provided by Cash et al., 2012. Per Cash et al., internet addiction is accompanied by changes in mood, preoccupation with the Internet and digital media, the inability to control the amount of time spent interfacing with digital technology, the need for more time to achieve a desired mood, withdrawal symptoms when not engaged, and a continuation of the behaviour despite family conflict, a diminishing social life and adverse work or academic consequences (Cash et al., 2012).

Exemplary diagnostic criteria for internet addiction proposed by Young, 1998 include presence of the following: (a) a preoccupation with the Internet (thinks about previous online activity or anticipates next online session), (b) need to use the Internet with increased amounts of time in order to achieve satisfaction, (c) repeatedly making unsuccessful efforts to control, cut back, or stop Internet use; (d) is restless, moody, depressed, or irritable when attempting to cut down or stop Internet use; (e) staying online longer than originally intended; (f) having jeopardised or risked the loss of a significant relationship, job, educational or career opportunity because of the Internet; (g) having lied to family members, therapist, or others to conceal the extent of involvement with the Internet; (h) and using the Internet as a way of escaping from problems or of relieving a dysphoric mood (e.g., feelings of helplessness, guilt, anxiety, depression) (Young, 1998). Per Young, subjects meeting five or more of the criteria are classified as dependent internet users, while those answering yes to less than five are classified as nondependent internet users (1998).

Internet addiction may be characterised as being mild, moderate, and severe. The severity of internet addiction may be determined by the internet addiction test (IAT), wherein a score between below 30 indicates a normal level of internet consumption, scores between 31 and 49 indicate mild level addiction, scores between 50 and 79 indicate moderate addiction, and scores of 80 or above indicate severe internet dependency (Samaha et al., 2018); or based on ordinary skill. In embodiments, a patient may have internet addiction characterised as being mild. In other embodiments, a patient may have internet addiction characterised as being moderate. In other embodiments, a patient may have internet addiction characterised as being severe. In embodiments, the Internet addiction scale (IAS), the Young of the Internet Addiction Questionnaire (YDQI), and the Chen's Internet addiction scale (CIAS) may also be utilised to assess the presence and/or severity of internet addiction.

In embodiments, the term “internet addiction patient” refers to a patient diagnosed with internet addiction, such as defined herein.

Herein, “reducing compulsive internet use” or “reduction in compulsive internet use” refers to reducing the amount or frequency of compulsive internet use, such as assessed by standardised screening tools like the IAS, YDQI, CIAS, and IAT. In embodiments, “reducing compulsive internet use” or “reduction in compulsive internet use” refers to a reduction in daily compulsive internet use, a reduction in the time spent compulsively using the internet per day, or a reduction in the frequency of compulsive internet use, such as a reduction in the percentage of compulsive internet use days in general, or a reduction in the percentage of heavy compulsive internet use days. In embodiments, “reducing compulsive internet use” or “reduction in compulsive internet use” refers to an increase in the time to any compulsive internet use or time to the first heavy compulsive internet use day.

Herein, “internet addiction abstinence” or “in abstinence from the internet” refers to not engaging in compulsive internet use. In embodiments, “promoting compulsive internet use abstinence” or “promotion of compulsive internet use abstinence” refers to help maintaining abstinence from compulsive internet use, in particular after at least 1 day of not engaging in compulsive internet use, for example, maintaining abstinence from compulsive internet use for a period of at least 1 day, at least 1 week, at least 1 month, or at least 1 year.

Herein, “internet addiction patient in abstinence from compulsive internet use” refers to a patient with internet addiction in abstinence from compulsive internet use for a period, for example, for at least 1 day, at least 1 week, at least 1 month, or at least 1 year.

Herein, “relapse into compulsive internet use” refers to engaging in compulsive internet use following a period of abstinence from compulsive internet use, for example following a period of compulsive internet use abstinence of at least 1 day, at least 1 week, at least 1 month, or at least 1 year.

Herein, “preventing relapse into compulsive internet use” refers to the prevention of engaging in compulsive internet use by an internet addiction patient after the patient has stopped exhibiting compulsive internet use, in particular after 1 day or more of not exhibiting compulsive internet use. In embodiments, the term encompasses a delay in the resumption of compulsive internet use as compared to the time to resumption by a patient that is not administered a compound of the invention using the disclosed methods. The delay in resumption can be for at least 1 day, at least 1 week, at least 1 month, or at least 1 year.

In embodiments, the patient with internet addiction is administered ketamine. In embodiments, the patient with internet addiction receives KAP. In embodiments, the patient with internet addiction undergoes a KAP treatment regimen. In embodiments, the administration of the ketamine is effective to treat internet addiction. In embodiments, the KAP is effective to treat internet addiction. In embodiments, the KAP treatment regimen is effective to treat internet addiction. In embodiments, HNK or norketamine is administered. In embodiments, HNK or norketamine is effective to treat internet addiction.

In embodiments, the administration of ketamine according to the disclosed methods will be effective to treat internet addiction as shown by a reduction in the severity of internet addiction. In embodiments, a patient who has internet addiction characterised as being mild when measured at baseline will no longer have a mild internet addiction. In embodiments, a patient who has internet addiction characterised as being moderate when measured at baseline will no longer have a moderate internet addiction. In embodiments, a patient who has internet addiction characterised as being severe when measured at baseline will no longer have a severe internet addiction. A reduction in the severity of internet addiction can be determined based on ordinary skill, such as using an assessment. For example, in embodiments, a patient who scores 30 or higher on the IAT when measured at baseline will have a reduction in IAT score of at least 4 points. In embodiments, a patient who scores 50 or higher on the IAT when measured at baseline will have a reduction in IAT score of at least 7 points. In embodiments, a patient who scores 80 points or higher on the IAT when measured at baseline will have a reduction in IAT score of at least 12 points.

In embodiments, the administration of ketamine according to the disclosed methods will be effective to treat internet addiction as shown by one or more of: (a) a reduction in compulsive internet use, (b) a reduction in drive or urge to engage in compulsive internet use, (c) a promotion of abstinence from compulsive internet use, (d) a prevention of relapse into compulsive internet use, (e) a reduction in at least one symptom of internet addiction.

In embodiments, administration of ketamine according to the disclosed methods will result in a measurable reduction in compulsive internet use or in the drive or urge to engage in compulsive internet use, for example as compared to a baseline determination made before such administration. In embodiments, the reduction in compulsive internet use is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from compulsive internet use or prevention of relapse into compulsive internet use, when measured from the start of treatment, such as the first administration. In embodiments, the promotion of abstinence from compulsive internet use results in abstinence for at least 1 day, at least 1 week, at least 1 month, or at least 1 year, from the start of treatment. In embodiments, the reduction in compulsive internet use, reduction in drive or urge to engage in compulsive internet use, promotion of abstinence from compulsive internet use, prevention of relapse into compulsive internet use, and reduction in at least one symptom of internet addiction lasts for at least 1 day, at least 1 week, at least 1 month, or at least 1 year.

In embodiments, administration of ketamine according to the disclosed methods will result in a measurable improvement in at least one symptom of internet addiction, for example as compared to a baseline determination made before such administration. In embodiments, the reduction in at least one symptom of internet addiction may be a decrease of one or more points of a patient completed self-assessment, as described herein, and/or a decrease of one or more points in a clinical outcome assessment or index score, such as the IAS. In embodiments, administration of ketamine according to the disclosed methods will result in an elimination of at least one symptom of internet addiction, for example as compared to a baseline determination made before such administration.

In embodiments, administration of ketamine according to the disclosed methods will result in the patient having a reduction in at least one symptom of internet addiction, including a preoccupation with the Internet, need to use the Internet with increased amounts of time in order to achieve satisfaction, repeatedly making unsuccessful efforts to control, cut back, or stop Internet use; is restless, moody, depressed, or irritable when attempting to cut down or stop Internet use; staying online longer than originally intended; having jeopardised or risked the loss of a significant relationship, job, educational or career opportunity because of the Internet; having lied to family members, therapist, or others to conceal the extent of involvement with the Internet; and using the Internet as a way of escaping from problems or of relieving a dysphoric mood.

In embodiments, administration of ketamine according to the disclosed methods will result in the patient having an elimination of at least one symptom of internet addiction, including a preoccupation with the Internet, need to use the Internet with increased amounts of time in order to achieve satisfaction, repeatedly making unsuccessful efforts to control, cut back, or stop Internet use; is restless, moody, depressed, or irritable when attempting to cut down or stop Internet use; staying online longer than originally intended; having jeopardised or risked the loss of a significant relationship, job, educational or career opportunity because of the Internet; having lied to family members, therapist, or others to conceal the extent of involvement with the Internet; and using the Internet as a way of escaping from problems or of relieving a dysphoric mood.

In embodiments, the patient with internet addiction has a comorbid psychiatric condition. In embodiments, treating the patient with internet addiction is also effective to treat one or more comorbid psychiatric conditions in the patient.

i. Pornography Addiction

In some aspects, the methods are useful in treating a patient with pornography addiction. Whether a patient has pornography addiction can be determined according to ordinary skill. For example, a patient may be determined to have pornography addiction based on the definition provided by Parker, 2021. Per Parker, pornography addiction is a compulsive need to view pornography despite negative consequences (2021).

Exemplary diagnostic criteria for pornography addiction include (a) you are consumed with thoughts of porn even when you are not actively watching it, (b) you view porn on your cell phone at work or in social situations where you might be seen a compulsion to watch more pornography, (c) you feel ashamed, guilty, or depressed about your porn viewing, (d) you continue to watch porn despite the harm it has had, is having, or may have on your relationships, work, or home life, (e) you experience reduced sexual satisfaction with partners when pornography is not involved, (f) you hide your porn and porn viewing from your partner and family members, (g) you get upset when asked to cut back on or stop looking at porn, (h) you lose track of time when viewing porn, and (i) you have tried to quit watching porn but have not been successful (Parker, 2021).

The Brief Pornography Screener (BPS) and/or the Problematic Porn Consumption Scale (PPCS) may be utilised to distinguish between problematic pornography use, and nonproblematic pornography consumption. A score on the BPS above 4 is considered problematic, while a score of above 76 on the PPCS is considered problematic.

In embodiments, the term “pornography addiction patient” refers to a patient diagnosed with pornography addiction, such as defined herein.

Herein, “reducing problematic pornography consumption” or “reduction in problematic pornography consumption” refers to reducing the amount or frequency of problematically consuming pornography, such as assessed by standardised screening tools like the BPS and PPCS. In embodiments, “reducing problematic pornography consumption” or “reduction in problematic pornography consumption” refers to a reduction in daily problematic pornography consumption, a reduction in the time spent problematically consuming pornography per day, or a reduction in the frequency of problematic pornography consumption, such as a reduction in the percentage of problematic pornography consumption days in general, or a reduction in the percentage of heavy problematic pornography consumption days. In embodiments, “reducing problematic pornography consumption” or “reduction in problematic pornography consumption” refers to an increase in the time to any problematic pornography consumption or time to the first heavy problematic pornography consumption day.

Herein, “pornography addiction abstinence” or “in abstinence from problematic pornography consumption” refers to not engaging in problematic pornography consumption. In embodiments, “promoting problematic pornography consumption abstinence” or “promotion of problematic pornography consumption abstinence” refers to help maintaining abstinence from problematic pornography consumption, in particular after at least 1 day of non-problematic pornography consumption, for example, maintaining abstinence from problematic pornography consumption for a period of at least 1 day, at least 1 week, at least 1 month, or at least 1 year.

Herein, “pornography addiction patient in abstinence from problematic pornography consumption” refers to a patient with pornography addiction in abstinence from problematic pornography consumption for a period, for example, for at least 1 day, at least 1 week, at least 1 month, or at least 1 year.

Herein, “relapse into problematic pornography consumption” refers to engaging in problematic pornography consumption following a period of abstinence from problematic pornography consumption, for example following a period of problematic pornography consumption abstinence of at least 1 day, at least 1 week, at least 1 month, or at least 1 year.

Herein, “preventing relapse into problematic pornography consumption” refers to the prevention of engaging in problematic pornography consumption by a pornography addiction patient after the patient has stopped problematic pornography consumption, in particular after 1 day or more of non-problematic pornography consumption. In embodiments, the term encompasses a delay in the resumption of problematic pornography consumption as compared to the time to resumption by a patient that is not administered a compound of the invention using the disclosed methods. The delay in resumption can be for at least 1 day, at least 1 week, at least 1 month, or at least 1 year.

In embodiments, the patient with pornography addiction is administered ketamine. In embodiments, the patient with pornography addiction receives KAP. In embodiments, the patient with pornography addiction undergoes a KAP treatment regimen. In embodiments, the administration of the ketamine is effective to treat pornography addiction. In embodiments, the KAP is effective to treat pornography addiction. In embodiments, the KAP treatment regimen is effective to treat pornography addiction. In embodiments, HNK or norketamine is administered. In embodiments, HNK or norketamine is effective to treat pornography addiction.

In embodiments, the administration of ketamine according to the disclosed methods will be effective to treat pornography addiction as shown by a reduction in the severity of pornography addiction. In embodiments, a patient who has pornography addiction characterised as being mild when measured at baseline will no longer have a mild pornography addiction. In embodiments, a patient who has pornography addiction characterised as being moderate when measured at baseline will no longer have a moderate pornography addiction. In embodiments, a patient who has pornography addiction characterised as being severe when measured at baseline will no longer have a severe pornography addiction A reduction in the severity of pornography addiction can be determined based on ordinary skill, such as using an assessment.

In embodiments, the administration of ketamine according to the disclosed methods will be effective to reduce the patient's pornography consumption. In embodiments, a patient who has pornography addiction characterised as being problematic by one or more of the BPS and/or the PPCS when measured at baseline will no longer have pornography addiction characterised as being problematic. In embodiments, a patient who scores 4 or higher on the BPS when measured at baseline will have a reduction in the BPS of at least 1 point. In embodiments, a patient who scores 76 or higher on the PPCS when measured at baseline will have a reduction in the PPCS of at least 10 points.

In embodiments, the administration of ketamine according to the disclosed methods will be effective to treat pornography addiction as shown by one or more of: (a) a reduction in pornography addiction, (b) a reduction in drive or urge to engage in problematic pornography consumption, (c) a promotion of abstinence from problematic pornography consumption, (d) a prevention of relapse into pornography addiction, (e) a reduction in at least one symptom of pornography addiction.

In embodiments, administration of ketamine according to the disclosed methods will result in a measurable reduction in problematic pornography consumption or in the drive or urge to engage in problematic pornography consumption, for example as compared to a baseline determination made before such administration. In embodiments, the reduction in problematic pornography consumption is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from problematic pornography consumption or prevention of relapse into problematic pornography consumption, when measured from the start of treatment, such as the first administration. In embodiments, the promotion of abstinence from problematic pornography consumption results in abstinence for at least 1 day, at least 1 week, at least 1 month, or at least 1 year, from the start of treatment. In embodiments, the reduction in problematic pornography consumption, reduction in drive or urge to engage in problematic pornography consumption, promotion of abstinence from problematic pornography consumption, prevention of relapse into problematic pornography consumption, and reduction in at least one symptom of pornography addiction lasts for at least 1 day, at least 1 week, at least 1 month, or at least 1 year.

In embodiments, administration of ketamine according to the disclosed methods will result in a measurable improvement in at least one symptom of pornography addiction, for example as compared to a baseline determination made before such administration. In embodiments, the reduction in at least one symptom of pornography addiction may be a decrease of one or more points of a patient completed self-assessment, as described herein, and/or a decrease of one or more points in a clinical outcome assessment or index score, such as the BPS, PPUS, and/or PPCS. In embodiments, administration of ketamine according to the disclosed methods will result in an elimination of at least one symptom of internet addiction, for example as compared to a baseline determination made before such administration.

In embodiments, administration of ketamine according to the disclosed methods will result in the patient having a reduction in at least one symptom of pornography addiction, including being consumed with thoughts of porn even when not actively watching it, viewing porn on their cell phone at work or in social situations where they might be seen, a compulsion to watch more pornography, feeling ashamed, guilty, or depressed about their porn viewing; continuing to watch porn despite the harm it has had, is having, or may have on their relationships, work, or home life, experiencing reduced sexual satisfaction with partners when pornography is not involved, hiding their porn and porn viewing from their partner and family members, getting upset when asked to cut back on or stop looking at porn, losing track of time when viewing porn, and trying to quit watching porn but not being successful.

In embodiments, administration of ketamine according to the disclosed methods will result in the patient having an elimination of at least one symptom of pornography addiction, including being consumed with thoughts of porn even when not actively watching it, viewing porn on their cell phone at work or in social situations where they might be seen, a compulsion to watch more pornography, feeling ashamed, guilty, or depressed about their porn viewing; continuing to watch porn despite the harm it has had, is having, or may have on their relationships, work, or home life, experiencing reduced sexual satisfaction with partners when pornography is not involved, hiding their porn and porn viewing from their partner and family members, getting upset when asked to cut back on or stop looking at porn, losing track of time when viewing porn, and trying to quit watching porn but not being successful.

In embodiments, the patient with pornography addiction has a comorbid psychiatric condition. In embodiments, treating the patient with pornography addiction is also effective to treat one or more comorbid psychiatric conditions in the patient.

j. Binge Eating Disorder

In some aspects, the methods are useful in treating a patient with binge eating disorder (BED). Whether a patient has binge eating disorder can be determined according to ordinary skill. For example, a patient may be determined to have binge eating disorder based on the definition in the ICD-11. The ICD-11 defines binge eating disorder as a discrete period of time (e.g., 2 hours) during which the individual experiences a loss of control over their eating behaviour and eats notably more or differently than usual. Loss of control over eating may be described by the individual as feeling like they cannot stop or limit the amount or type of food eaten; having difficulty stopping eating once they have started; or giving up even trying to control their eating because they know they will end up overeating (WHO, 2022).

ICD-11 diagnostic criteria for binge eating disorder include (a) frequent, recurrent episodes of binge eating (e.g., once a week or more over a period of 3 months). Binge eating is defined as a discrete period of time (e.g., 2 hours) during which the individual experiences a loss of control over their eating behaviour and eats notably more or differently than usual. Loss of control over eating may be described by the individual as feeling like they cannot stop or limit the amount or type of food eaten; having difficulty stopping eating once they have started; or giving up even trying to control their eating because they know they will end up overeating. (b) The binge eating episodes not regularly being accompanied by inappropriate compensatory behaviours aimed at preventing weight gain, (c) the symptoms and behaviours not being better accounted for by another medical condition (e.g., Prader-Willi Syndrome) or mental disorder (e.g., a Depressive Disorder) and are not due to the effects of a substance or medication on the central nervous system, including withdrawal effects, and (d) there is marked distress about the pattern of binge eating or significant impairment in personal, family, social, educational, occupational or other important areas of functioning (WHO, 2022).

The DSM-V defines binge eating disorder as recurrent episodes of binge eating that is defined as an amount of food that is definitely larger than most people would eat in a similar period of time under similar circumstances accompanied by a sense of lack of control over eating during the episode (APA, 2013).

Per the DSM-V, an episode of binge eating is characterised by both of the following: (1) eating, in a discrete period of time (e.g. —within any 2-hour period), an amount of food that is definitely larger than what most people would eat in a similar period of time under similar circumstances, (2) a sense of lack of control over eating during the episode (e.g. —a feeling that one cannot stop eating or control what or how much one is eating). The binge eating episodes are associated with at least 3 of the following: (a) eating much more rapidly than normal, (b) eating until feeling uncomfortably full, (c) eating large amounts of food when not feeling physically hungry, (d) eating alone because of being embarrassed by how much one is eating, (e) feeling disgusted with oneself, depressed, or very guilty after overeating. In addition, marked distress regarding binge eating is present, the binge eating occurs, on average, at least once a week for three months; and the binge eating is not associated with the recurrent use of inappropriate compensatory behaviours (such as purging) and does not occur exclusively during the course Bulimia Nervosa or Anorexia Nervosa.

Binge eating disorder may be characterised as being mild, moderate, and severe. The severity of binge eating disorder may be determined by the Binge Eating Scale (BES), wherein a score below 17 indicates no binge eating disorder, a score of between 18-20 indicates mild binge eating disorder, a score of between 21-27 indicates moderate binge eating disorder, and a score above 27 indicates severe binge eating disorder (Grupski et al., 2013), or based on ordinary skill. In embodiments, a patient may have binge eating disorder characterised as being mild. In other embodiments, a patient may have binge eating disorder characterised as being moderate. In other embodiments, a patient may have binge eating disorder characterised as being severe.

In embodiments, “binge eating disorder patient” (or “patient with binge eating disorder”) refers to a patient diagnosed with binge eating disorder, such as defined herein.

Herein, “reducing binge eating” or “reduction in binge eating” refers to reducing the amount or frequency of binge eating, such as assessed by standardised screening tools like the BES. In embodiments, “reducing binge eating” or “reduction in binge eating” refers to a reduction in daily binge eating, a reduction in the time spent binge eating per day, or a reduction in the frequency of binge eating, such as a reduction in the percentage of binge eating days in general, or a reduction in the percentage of heavy binge eating days. In embodiments, “reducing binge eating” or “reduction in binge eating” refers to an increase in the time to any binge eating or time to the first heavy binge eating day.

Herein, “binge eating abstinence” or “in abstinence from binge eating” refers to not engaging in binge eating. In embodiments, “promoting binge eating abstinence” or “promotion of binge eating abstinence” refers to help maintaining abstinence from binge eating, in particular after at least 1 day of not binge eating, for example, maintaining abstinence from binge eating for a period of at least 1 day, at least 1 week, at least 1 month, or at least 1 year.

Herein, “binge eating disorder patient in abstinence from binge eating” refers to a patient with binge eating disorder in abstinence from binge eating for a period, for example, for at least 1 day, at least 1 week, at least 1 month, or at least 1 year.

Herein, “relapse into binge eating” refers to engaging in binge eating following a period of abstinence from binge eating, for example following a period of binge eating abstinence of at least 1 day, at least 1 week, at least 1 month, or at least 1 year.

Herein, “preventing relapse into binge eating” refers to the prevention of engaging in binge eating by a binge eating disorder patient after the patient has stopped binge eating, in particular after 1 day or more of not binge eating. In embodiments, the term encompasses a delay in the resumption of binge eating as compared to the time to resumption by a patient that is not administered a compound of the invention using the disclosed methods. The delay in resumption can be for at least 1 day, at least 1 week, at least 1 month, or at least 1 year.

In embodiments, the patient with binge eating disorder is administered ketamine. In embodiments, the patient with binge eating disorder receives KAP. In embodiments, the patient with binge eating disorder undergoes a KAP treatment regimen. In embodiments, the administration of the ketamine is effective to treat binge eating disorder. In embodiments, the KAP is effective to treat binge eating disorder. In embodiments, the KAP treatment regimen is effective to treat binge eating disorder. In embodiments, HNK or norketamine is administered. In embodiments, HNK or norketamine is effective to binge eating disorder.

In embodiments, the administration of ketamine according to the disclosed methods will be effective to treat binge eating disorder as shown by a reduction in the severity of binge eating disorder. In embodiments, a patient who has binge eating disorder characterised as being mild when measured at baseline will no longer have a mild binge eating disorder. In embodiments, a patient who has binge eating disorder characterised as being moderate when measured at baseline will no longer have a moderate binge eating disorder. In embodiments, a patient who has binge eating disorder characterised as being severe when measured at baseline will no longer have a severe binge eating disorder. A reduction in the severity of binge eating disorder can be determined based on ordinary skill, such as using an assessment. For example, in embodiments, a patient who scores 18 or higher on the BES when measured at baseline will have a reduction in BES score of at least 2 points. In embodiments, a patient who scores 21 or higher on the BES when measured at baseline will have a reduction in BES score of at least 3 points. In embodiments, a patient who scores 27 or higher on the BES when measured at baseline will have a reduction in BES score of at least 4 points.

In embodiments, the administration of ketamine according to the disclosed methods will be effective to treat binge eating disorder as shown by one or more of: (a) a reduction in binge eating, (b) a reduction in drive or urge to engage in binge eating, (c) a promotion of abstinence from binge eating, (d) a prevention of relapse into binge eating, (e) a reduction in at least one symptom of binge eating disorder.

In embodiments, administration of ketamine according to the disclosed methods will result in a measurable reduction in binge eating or in the drive or urge to engage in binge eating, e.g., as compared to a baseline determination made before such administration. In embodiments, the reduction in binge eating is at least 15%, including 20%, 25%, 30%, 40%, 50%, 60%, 70%, 80%, 90%, up to and including 100%, when measured from baseline.

In embodiments, administration of ketamine according to the disclosed methods will result in a durable promotion of abstinence from binge eating or prevention of relapse into binge eating, when measured from the start of treatment, such as the first administration. In embodiments, the promotion of abstinence from binge eating results in abstinence for at least 1 day, at least 1 week, at least 1 month, or at least 1 year, from the start of treatment. In embodiments, the reduction in binge eating, reduction in drive or urge to engage in binge eating, promotion of abstinence from binge eating, prevention of relapse into binge eating, and reduction in at least one symptom of binge eating disorder lasts for at least 1 day, at least 1 week, at least 1 month, or at least 1 year.

In embodiments, administration of ketamine according to the disclosed methods will result in the patient having a reduction in at least one symptom of binge eating disorder, including frequent, recurrent episodes of binge eating, feeling like they cannot stop or limit the amount or type of food eaten; having difficulty stopping eating once they have started; giving up even trying to control their eating because they know they will end up overeating; marked distress about the pattern of binge eating or significant impairment in personal, family, social, educational, occupational or other important areas of functioning; eating, in a discrete period of time an amount of food that is definitely larger than what most people would eat in a similar period of time under similar circumstances, a sense of lack of control over eating during the episode; eating much more rapidly than normal, eating until feeling uncomfortably full, eating large amounts of food when not feeling physically hungry, eating alone because of being embarrassed by how much one is eating, and feeling disgusted with oneself, depressed, or very guilty after overeating.

In embodiments, administration of ketamine according to the disclosed methods will result in the patient having an elimination of at least one symptom of binge eating disorder, including frequent, recurrent episodes of binge eating, feeling like they cannot stop or limit the amount or type of food eaten; having difficulty stopping eating once they have started; giving up even trying to control their eating because they know they will end up overeating; marked distress about the pattern of binge eating or significant impairment in personal, family, social, educational, occupational or other important areas of functioning; eating, in a discrete period of time an amount of food that is definitely larger than what most people would eat in a similar period of time under similar circumstances, a sense of lack of control over eating during the episode; eating much more rapidly than normal, eating until feeling uncomfortably full, eating large amounts of food when not feeling physically hungry, eating alone because of being embarrassed by how much one is eating, and feeling disgusted with oneself, depressed, or very guilty after overeating.

In embodiments, administration of ketamine according to the disclosed methods will result in the patient no longer having episodes of binge eating, having distress about the pattern of binge eating or significant impairment in personal, family, social, educational, occupational or other important areas of functioning; eating rapidly, feeling uncomfortably full, eating large amounts of food when not feeling physically hungry, eating alone because of being embarrassed by how much they are eating, feeling disgusted with oneself, depressed, or very guilty after overeating; having distress regarding binge eating, and having episodes of binge eating at least once per week.

In embodiments, the patient with binge eating disorder has a comorbid psychiatric condition. In embodiments, treating the patient with binge eating disorder is also effective to treat one or more comorbid psychiatric conditions in the patient.

### B. Ketamine

Ketamine is the compound 2-(2-chlorophenyl)-2-(methylamino)-cyclohexanone. A chiral compound, ketamine has S(+) and R(−) stereoisomers. Herein, the S(+) stereoisomer, S-ketamine (esketamine), refers to the compound (2S)-2-(2-Chlorophenyl)-2-(methylamino) cyclohexanone, or a pharmaceutically acceptable salt thereof, as well as a pharmaceutical composition comprising, consisting essentially of, or consisting of said compound, for example in pure or substantially pure enantiomeric form. Herein, the R(−) stereoisomer, R-ketamine (arketamine), refers to the compound (2R)-2-(2-Chlorophenyl)-2-(methylamino) cyclohexanone, or a pharmaceutically acceptable salt thereof, as well as a pharmaceutical composition comprising, consisting essentially of, or consisting of said compound, for example in pure or substantially pure enantiomeric form. When racemic ketamine is referred to herein, it will include racemic (±)-ketamine, a pharmaceutically acceptable salt thereof (e.g., racemic ketamine HCl), as well as a pharmaceutical composition comprising, consisting essentially of, or consisting of racemic ketamine.

Herein, and unless context indicates otherwise (e.g., where it is clear that racemic ketamine or a ketamine stereoisomer is intended) “ketamine” is used as shorthand and encompasses both racemic and non-racemic ketamine, including a pure or substantially pure ketamine enantiomer (i.e., pure or substantially pure S(+) or R(−) enantiomer), an enantiomerically enriched mixture of ketamine, a ketamine metabolite, or a ketamine analog, including pharmaceutically acceptable salts thereof, and including combinations of any of the foregoing. For enantiomerically enriched compositions or compositions of individual ketamine enantiomers, as well as for ketamine metabolites and ketamine analogs, dosage may be adjusted based on known differences in potency or effect, and can be done so based on the teachings herein in combination with the general knowledge in the art.

In embodiments, “R-ketamine” shall mean a composition comprising only the R(−) stereoisomer, and “S-ketamine” shall mean a composition comprising only the S(+) stereoisomer (and “enantiomeric ketamine” shall mean either or both). In embodiments, these terms are understood to mean a substantially pure or pure enantiomer, for example R-ketamine also shall mean a mixture of R(−)- and S(+)-ketamine in an amount of R:S of greater than 90%, greater than 95%, greater than 96%, preferably greater than 97%, more preferably greater than 98%, more preferably greater than 99%, more preferably greater than 99.5%, and most preferably greater than 99.9%, and likewise with regard to S-ketamine.

Also within the definition of “ketamine,” in some embodiments, are its known derivatives and analogs, including such “designer drug” or “research chemical” ketamine “analogs” such as methoxetamine (2-MeO-2-deschloroketamine, MXE), 3-MeO-PCE, KEA-1010, N-ethyldeschloroketamine (2′-Oxo-PCE, O-PCE), 2-fluoro-deschloroketamine [2-(2-fluorophenyl)-2-methylamino-cyclohexanone] (2-FDCK), deschloro-ketamine (2-phenyl-2-methylamino-cyclohexanone), DXE (2′-Oxo-PCM, aka DCK), alkyne-norketamine (A-NK), and the like; and any of the 12 HNK metabolites formed from the metabolism of ketamine in vivo, including any of the stereoselective metabolites of R- or S-ketamine, such as (R,S)-norketamine (NK), (R,S)-dehydronorketamine, hydroxyketamines, and hydroxynorketamines (HNKs), including (2S,6S;2R,6R)-HNK, (2R,4R;2S,4S-2S,6R;2R,6S)-HNK, and (2R,4S;2S,4R-2S,5S;2R,5R)-HNK (Farmer, 2020). In embodiments, ketamine also refers to such additional arylcyclohexylamine derivatives as are disclosed in, e.g., WO2022/047256A1, US2022/0041540A1, and WO2021/255737A1.

Ketamine prodrugs are also within the scope of the “ketamine” of the invention. The term “prodrug” refers to a precursor of a biologically active pharmaceutical agent. Prodrugs undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety, such as a glycoside or acetyl group, to form the biologically active pharmaceutical agent. Other examples include the addition of hydroxyl groups (Tsujikawa et al. 2011. Xenobiotica, 41(7), 578-584; Yamamoto et al. 1984. Xenobiotica, 14(11), 867-875), acyloxyalkoxycarbonyl derivatives, amino acids, or peptides (Vig et al. 2013. Advanced Drug Delivery Reviews, 65(10), 1370-1385), which can be added to an amine, and can be removed within the body by chemical reactions or enzymes, but other prodrugs and precursors, at the amine and other sites, should be understood to be within the scope of the invention (Simplicio, Clancy, & Gilmer. 2008. Molecules, 13(3), 519-547; Shah, Chauhan, Chauhan, & Mishra (Eds.). 2020. Recent Advancement in Prodrugs. CRC Press).

Ketamine prodrugs include compounds that are transformed in various organs or locations in the body (e.g., liver, kidney, G.I., lung, tissue) to release the active compound. For example, liver prodrugs will include active compounds conjugated with a polymer or chemical moiety that is not released until acted upon by liver cytochrome enzymes; CYP metabolism includes dealkylation, dehydrogenation, reduction, hydrolysis, oxidation, and the breakdown of aromatic rings. Kidney prodrugs will include active compounds conjugated to L-gamma-glutamyl or N-acetyl-L-gamma glutamic moieties so that they are metabolised by gamma-glutamyl transpeptidase before they are bioactive; alternatively, they may be conjugated to alkylglucoside moieties to create glycosylation-based prodrugs. Digestive or G.I. prodrugs will include those where an active compound is, e.g., formulated into microspheres or nanospheres that do not degrade until the spheres are subjected to an acidic pH; formulated with an amide that will resist biochemical degradation until colonic pH is achieved; or conjugated with a linear polysaccharide such as pectin that will delay activation until the combination reaches the bacteria in the colon.

Besides these exemplary prodrug forms, many others will be known to those of skill. For example, additional prodrugs include compounds with biologically labile or cleavable (protecting) groups on a functional moiety of the active compound. Prodrugs include compounds that can be oxidised, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, which are incorporated herein by reference. Conventional procedures for the selection and preparation of suitable prodrugs are described, e.g., in “Design of Prodrugs” Ed. H. Bundgaard, Elsevier, 1985.

### A. Pharmaceutical Compositions

In some aspects, ketamine is administered in the disclosed methods as a pharmaceutical composition. “Pharmaceutical compositions” comprise disclosed compound(s) together in an amount (for example, in a unit dosage form) with a pharmaceutically acceptable carrier, diluent, or excipient. It will be understood that some embodiments do not have a single carrier, diluent, or excipient alone, but have multiple carriers, diluents, and/or excipients. Compositions can be prepared by standard pharmaceutical formulation techniques such as disclosed in Remington: Science and Practice of Pharmacy (2005) 21th ed., Mack Pub. Co., Easton, Pa.; The Merck Index (1996) 12th ed., Merck Pub. Group, Whitehouse, N.J.; Pharm. Principles of Solid Dosage Forms (1993), Technomic Pub. Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y, pp. 253-315). “Pharmaceutically acceptable,” as used in connection with one or more ingredients, means the ingredients are generally safe and, within the scope of sound medical judgment, suitable for use in contact with the cells of humans and other animals without undue toxicity, irritation, allergic response, or complication, and commensurate with a reasonable risk/benefit ratio.

Pharmaceutical compositions comprising ketamine (“ketamine compositions”) can be formulated into any suitable dosage form, such as aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilised formulations, tablets, capsules, pills, powders, pulsatile release formulations, multi-particulate formulations, immediate release, controlled release, sustained release, extended release, and modified release formulations, and mixed immediate release and controlled release formulations.

In embodiments, a ketamine composition is formulated in a unit dosage form. “Unit dosage form” refers to a physically discrete unit suited as unitary dosages for the patient to be treated, each unit containing a predetermined quantity of active agent calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilised state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a patient for an extended or brief period of time.

In embodiments, a ketamine composition is formulated in a pharmaceutically acceptable oral dosage form, including oral solid dosage forms and oral liquid dosage forms. In embodiments, the compositions are formulated as a pharmaceutically acceptable oral solid dosage form, including lozenges, troches, tablets, capsules, caplets, powders, pellets, multiparticulates, beads, spheres, capsules, pills, and/or any combinations thereof. Oral solid dosage forms may be formulated as immediate release, controlled release, sustained release, extended release, or modified release formulations.

In embodiments, solid dosage forms may comprise pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. In embodiments, solid dosage forms may comprise pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s), including preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the pharmaceutical formulation, and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid (ALA).

In embodiments of modified release formulations, the plasma half-life compared to the plasma half-life of an immediate release formulation is greater by at least 5%, at least 10%, at least 15%, at least 20%, at least 25%, at least 30%, at least 35%, at least 50%, at least 75%, at least 100%, or values in between. In embodiments of modified release formulations, the formulations are designed to result in a comparable area under the curve, or AUCO₂₄, and a similar safety and efficacy profile, but having a delayed time to peak concentration (tₘₐₓ) of at least 5%, at least 10%, at least 15%, at least 20%, at least 25%, at least 30%, at least 35%, at least 50%, at least 75%, at least 100%, or values in between. In embodiments, a formulation is designed to be a product with a specific time course based on an optimum therapeutic window, such as less than about 30 minutes, about 30 minutes, about 45 minutes, about 60 minutes, about 90 minutes, about 2 hours, about 3 hours, about 4 hours, and greater than 4 hours, including lengths of time in between.

In embodiments, a ketamine composition is formulated as a pharmaceutically acceptable oral liquid dosage form. Non-limiting examples of oral liquid dosage forms include tinctures, drops, emulsions, syrups, elixirs, suspensions, dispersions, and solutions, and the like. In embodiments, oral liquid dosage forms may be formulated with any pharmaceutically acceptable excipient known to those of skill for the preparation of liquid dosage forms, and with solvents, diluents, carriers, excipients, and the like, chosen as appropriate to the solubility and other properties of the active agents and other ingredients.

In embodiments, a ketamine composition is formulated as an effervescent powder.

In embodiments, a ketamine composition is formulated in a pharmaceutically acceptable transdermal application, capable of being administered transdermally. Non-limiting examples of transdermal formulations include ointments, creams, suspensions, lotions, pastes, gels, sprays, foams, oils, and the like, and any combination thereof.

In embodiments, a ketamine composition is formulated for subcutaneous, intravenous, intra-arterial, intraperitoneal, intraosseous, intramuscular, intrathecal, or intracerebroventricular injection (“injectable formulations”). In embodiments, injectable formulations may be prepared by dissolving, suspending, or emulsifying the active agent(s) in an aqueous or nonaqueous solvent, non-limiting examples of which include oils, such as vegetable oil, synthetic aliphatic acid glycerides, and esters of higher aliphatic acids or propylene glycol; and may also comprise additives such as solubilizers, stabilizers, and suspending, preserving, wetting, emulsifying, dispensing, and isotonic agents.

Injectable formulations may comprise additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like, and may comprise isotonic agents, such as sugars, sodium chloride, and the like. Prolonged drug absorption of the injectable pharmaceutical form can be brought about by the use of agents delaying absorption, such as aluminum monostearate and gelatin. Injectable formulations designed for extended-release via SC or IM injection can avoid first-pass metabolism and lower dosages of ketamine will be necessary to maintain desired plasma levels. In such formulations, the particle size and the range of the particle sizes of the active agent(s) can be used to control the release of the agent(s) by controlling the rate of dissolution in fat or muscle.

In embodiments, a ketamine composition is formulated in a pharmaceutically acceptable nanostructured formulation, such as a nanoemulsion, a nanocapsule, a nanoparticle conjugate, or a nano-encapsulated oral, sublingual, buccal, or nasal spray. In embodiments, nanostructured formulations may be prepared by reference to the general knowledge of the art (see, e.g., Jaiswal 2015).

By way of non-limiting and merely suggestive examples, the following formulations may be used in the disclosed methods.

### Example 1: Injectable formulation

A formulation for injection (e.g., SC, IM, IP, or IV delivery) is prepared as follows:

| Ingredient | Amount | |
|---|---|---|
| Ketamine | 50 | g |
| DMSO | 5 | mL |
| TEG | 500 | mL |
| Saline (1% cremophor) | To 1 | L |

Ketamine is dissolved in dimethyl sulphoxide (DMSO) in proportions of 50 g to 5 mL. Solution is brought to 37° C. and vortexed for 3-5 minutes. Tetraethylene glycol (TEG) in the amount of 500 mL is added, and solution is returned to 37′ C and vortexed again for 3-5 mins. Solution is mixed 1:1 with saline containing 1% cremophor to prevent precipitation. Final solution will be at 500 mg/10 mL ketamine in 49.5% TEG, 49.5% saline, 0.50% DMSO, and 0.50% cremophor. Injection may be by any suitable means, e.g., bolus injection (IM, SC, IP, etc.), IV infusion, or subcutaneous infusion, for example using a drug delivery device comprising a reservoir and a pump mechanism, configured for subcutaneous administration, and which may optionally comprise a user interface or be coupled to a device with a user interface such as a smartphone (e.g., a subcutaneous continuous infusion pump).

### Example 2: Alternate Injectable Solutions

An injectable formulation is prepared as follows:

| Ingredient | Amount | |
|---|---|---|
| Ketamine | 500 | mg |
| Isotonic saline | 5 | mL |

| Ingredient | Amount | |
|---|---|---|
| Ketamine | 500 | mg |
| Isotonic saline | 10 | mL |

Ketamine is dissolved in appropriate solvent as will be understood by those ofordinary skill; isotonic saline is used in this Example, but it will be appreciated that other solvents may be used, and additional active or inactive ingredients such as preservatives may be added, as in Example 1, as otherwise described above, and within the general knowledge of the art.

### Example 3: Intranasal Delivery Form

A nasal spray formulation for intranasal delivery is prepared as follows:

| Ingredient | Amount | |
|---|---|---|
| Ketamine | 1 | wt % |
| DMSO | 50 | μL |
| MCT | 5 | mL |
| Saline (1% cremophor) | To 10 | mL |

Solution at 10 mg/mL ketamine in 49.5% MCT, 49.5% saline, 0.5% DMSO, and 0.5% cremophor is prepared, as above (with MCT in place of TEG), for use in nasal spray devices.

### Example 4: Dry Powder for Insufflation

A dry powder inhaler formulation is prepared comprising the following components:

| Ingredient | Weight % |
|---|---|
| Ketamine | 5 |
| Lactose | 95 |

Ketamine is combined with lactose (as a carrier) and prepared for use with a dry powder inhaling appliance, or as in U. S. Pub. No. US2015/0367091A1 and references cited therein.

### Example 5: Cut Matrix Sublingual or Buccal Lozenges

Lozenges, made as a single matrix and then cut to size, are made as follows:

| Ingredient | Amount (mg/lozenge) |
|---|---|
| Ketamine | 25.0 |
| Glycerol | 200.0 |
| Water | 142.0 |
| Sodium Citrate | 5.0 |
| Polyvinyl Alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and ketamine is slowly admixed. The homogenous mixture is poured into forms made of an inert material to produce a drug-containing diffusion matrix having a thickness of about 2-4 mm. This diffusion matrix is then cut to form individual lozenges having the appropriate size.

### Example 6: Individually Formed Sublingual or Buccal Lozenges

Lozenges, made from individual forms or molds, are made as follows:

| Ingredient | Amount (each lozenge) | |
|---|---|---|
| Ketamine | 100.0 | mg |
| Silica gel powder | 350.0 | mg |
| Citric acid powder | 400.0 | mg |
| Acacia powder | 600.0 | mg |
| Flavoring (optional) | 100.0 | mg |
| Polyethylene glycol | 1.0 | g |

Inactive ingredients are admixed together by continuous stirring and maintaining temperature at about 90° C. When the PEG has melted and the other ingredients have gone into solution, the solution is cooled to about 50-55° C. and ketamine is slowly admixed. The homogenous mixture is poured into separate molds and allowed to cool. Reference is made to U.S. Pat. No. 10,034,832 and the Examples therein, the entirety of which is incorporated herein.

It should be readily appreciated that the above formulation examples are illustrative only. Accordingly, any of the compounds may be substituted with the same compound in a different amount. Additionally, reference to particular compounds is merely illustrative, and both active and inactive compounds in any Example may be substituted by other compounds.

For example, “ketamine” in EXAMPLES 1-6 above should be construed to include racemic ketamine, an enantiomerically enriched composition of ketamine, or an individual enantiomer (i.e., R-ketamine or S-ketamine) of particular purity, as well as a ketamine metabolite or a ketamine analog, or a combination of any of the foregoing, and further including substitution of any compound by its ion, free base, or salt form, or by a polymorph (with modifications to the formulation and dosage amounts made according to the teachings herein and ordinary skill), such as defined and discussed above.

Further, compositions within the scope of the invention should be understood to be open-ended and may include additional active or inactive compounds and ingredients.

Finally, the type of formulation employed for the administration of the ketamine compound(s) employed in the disclosed methods generally may be dictated by the specific compound(s) employed (e.g., whether it is racemic ketamine, a single enantiomer, or an enantiomerically-enriched composition, or a metabolite or analog), the type of pharmacokinetic profile desired from the route of administration and the compound(s), and the state of the patient. It will be readily appreciated that any of the above embodiments and classes of embodiments can be combined to form additional embodiments and formulations.

The ketamine compositions can be formulated for administration by a variety of routes. Non-limiting examples of routes of administration include enteral administration, such as oral, sublingual, buccal, and rectal administration; parenteral administration, including bolus injection or continuous infusion, intravenous (IV), intra-arterial, intraperitoneal (IP), intraosseous, intramuscular (IM), intrathecal, intracerebroventricular, vaginal, ocular, nasal, cutaneous, topical, otic, ocular, transdermal, and subcutaneous (SC) administration.

In embodiments, the ketamine compositions can be effectively administered as oral solid and oral liquid dosage forms; sublingually or buccally; as injections, including intravenously, intra-arterially, intraperitoneally, intraosseously, intramuscularly, intrathecally, and intracerebroventricularly; rectally, vaginally, ocularly, nasally, cutaneously, topically, oticly, ocularly, transdermally, and subcutaneously.

In some aspects are methods of administering a pharmaceutical composition. In embodiments, the methods comprise the administration of a therapeutically effective amount of a pharmaceutical composition for the treatment of a behavioural addiction. Administration of a pharmaceutical composition in a “therapeutically effective amount” or an “effective amount” refers to an amount of an active agent sufficient to provide the desired therapeutic effect, for example, reducing one or more of the symptoms of the disease or condition being treated. The term “therapeutically effective amount” additionally includes, for example, a prophylactically effective amount. The effective amount may vary depending upon the patient and the disorder being treated, e.g., the weight, age, and metabolic profile of the patient, the severity of the disorder, the manner of administration, and the like, all of which can readily be determined by one of skill. “Therapeutic effect” means the response(s) in the patient after treatment that are judged to be desirable and beneficial. Hence, depending on the disorder to be treated, and depending on the pharmaceutical composition administered, those responses shall differ, but would be readily understood by those of skill.

In some embodiments, a pharmaceutical composition further comprises an additional active agent. In embodiments, the additional active agent is any of an opioid antagonist (e.g., nalmefene, naltrexone), a CB-1 antagonist (e.g., rimonabant), a CRH1 receptor antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxcarbazepine, valproic acid, zonisamide), a voltage-dependent calcium channel agonist (e.g., gabapentin, pregabalin), an α7 nicotinic acetylcholine receptor agonist (e.g., varenicline), an α1 adrenoceptor antagonist (e.g., doxazosin, prazosin), a glucocorticoid receptor antagonist (e.g., mifepristone), an al adrenoceptor agonist (e.g., guanfacine), an AChE inhibitor (e.g., citicoline), a dopamine D2 receptor antagonist (e.g., tiapride), an α2 adrenoceptor agonist (e.g., clonidine), an NMDA receptor antagonist (e.g., acamprosate), and an aldehyde dehydrogenase inhibitor (e.g., disulfiram), or pharmaceutically acceptable salts thereof.

In embodiments, an additional active agent is a serotonergic agent. Herein, a “serotonergic agent” refers to any compound that binds to, blocks, or otherwise influences (e.g., via an allosteric reaction) activity at one or more serotonin receptors, including any one or more serotonin receptor subtypes. In embodiments, a serotonergic agent binds to a serotonin receptor. In embodiments, a serotonergic agent indirectly affects a serotonin receptor, e.g., via interactions affecting the reactivity of other molecules at the serotonin receptor. In embodiments, a serotonergic agent is an agonist, e.g., a compound activating a serotonin receptor, such as 5-HT_{1A} receptor partial agonist aripiprazole. In embodiments, a serotonergic agent is an antagonist, e.g., a compound binding but not activating a serotonin receptor, e.g., blocking a receptor, such as a 5-HT₃ antagonist ondansetron, and 5-HT_{2A} receptor antagonists quetiapine, olanzapine, and mirtazapine. In embodiments, a serotonergic agent is an effector molecule, e.g., a compound binding to an enzyme for allosteric regulation. In embodiments, a serotonergic agent acts (either directly or indirectly) at more than one type of receptor, including receptors other than serotonergic or other monoaminergic receptors. In embodiments, a serotonergic agent blocks the serotonin transporter (SERT) and results in an elevation of the synaptic concentration of serotonin, and an increase of neurotransmission (such as SERT inhibitor duloxetine). In embodiments, a serotonergic agent is a serotonin uptake or reuptake inhibitor, such as trazodone. In embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In embodiments, a serotonergic agent inhibits the activity of one or both monoamine oxidase enzymes, resulting in an increase in concentrations of serotonin and an increase in neurotransmission. In embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

In embodiments, the additional active agent is selected to provide an additional therapeutic effect, such as antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, entactogenic, empathogenic, entheogenic, euphoric, psychedelic, sedative, and stimulant effects.

In embodiments, the additional active agent may be any of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, entactogens and empathogens, entheogens, psychedelics, monoamine oxidase inhibitors, sedatives, stimulants, and vitamins. These active agents may be in ion, freebase, or salt form, include polymorphs, and may be isomers.

In embodiments, the additional active agent is selected to provide synergistic effects. In embodiments, “synergistic effects” will be understood to include increases in potency, bioactivity, bioaccessibility, bioavailability, or therapeutic effect, that are greater than the additive contributions of the components acting alone, and/or are greater than the contribution of the isolated compounds on their own. Numerous methods known to those of skill in the art exist to determine whether there is synergy as to a particular effect, i.e., whether, when two or more components are mixed together, the effect is greater than the sum of the effects of the individual components when applied alone, thereby producing “1+1>2.” One such method is the isobologram analysis (or contour method) (Huang et al. 2019).

### C. Dose and Dosage

In some aspects are disclosed methods for using therapeutically effective amounts of the pharmaceutical compositions of the invention in a mammal, and preferably a human. Such methods include those for treating a behavioural addiction. In embodiments, pharmaceutical compositions comprise therapeutically effective amounts (doses) of ketamine.

In embodiments, therapeutically effective doses range from between about 0.1 mg/kg to about 1.4 mg/kg. In embodiments, therapeutically effective doses will be determined based on, e.g., the patient's needs, prior experience, and degree of clinical response, or otherwise based on clinical or medical judgment. TABLE 1 illustrates exemplary doses.

TABLE 1
Exemplary Ketamine Injection Dosing

| | | | Using | Using |
|---|---|---|---|---|
| | Ketamine dose | | 500 mg/5 mL | 500 mg/10 mL |
| | required to | Equivalent to: | bottle: | bottle: |
| Type of | achieve | mg/kg | Volume of | Volume of |
| psychotherapy | desired type of | (in a typical | liquid to be | liquid to be |
| desired: | psychotherapy | 70 kg patient) | injected IM | injected IM |
| Psycholytic dose. | 10 mg | 0.1 mg/kg | 0.1 mL | 0.2 mL |
| Talking therapy is | 20 mg | 0.3 mg/kg | 0.2 mL | 0.4 mL |
| possible during | 30 mg | 0.4 mg/kg | 0.3 mL | 0.6 mL |
| drug experience. | 40 mg | 0.6 mg/kg | 0.4 mL | 0.8 mL |
| Full dissociative/ | 50 mg | 0.7 mg/kg | 0.5 mL | 1.0 mL |
| psychedelic dose. | 60 mg | 0.9 mg/kg | 0.6 mL | 1.2 mL |
| Non-verbal session. | 70 mg | 1.0 mg/kg | 0.7 mL | 1.4 mL |
| Engaging in talking | 80 mg | 1.1 mg/kg | 0.8 mL | 1.6 mL |
| therapy during drug | 90 mg | 1.2 mg/kg | 0.9 mL | 1.8 mL |
| experience unlikely. | 100 mg | 1.4 mg/kg | 1.0 mL | 2.0 mL |

In embodiments where a pharmaceutical composition comprises ketamine, it may be present in an amount so that a single dose is (in a mg dose amount calculated based on the kilogram weight of the patient), e.g., 0.1 mg/kg or less, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1.0 mg/kg, at least 1.1 mg/kg, at least 1.2 mg/kg, at least 1.3 mg/kg, or at least 1.4 mg/kg, as well as amounts within these ranges.

In some embodiments where a pharmaceutical composition comprises ketamine, the ketamine may be present in an amount so that a single dose is (in a mg dose amount to be administered to the patient), e.g., 1 mg or less, at least 1 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, or at least 200 mg (including at least 250 mg, at least 300 mg, at least 350 mg, at least 400 mg, at least 450 mg, at least 500 mg, at least 550 mg, and at least 600 mg), as well as amounts within these ranges. In some preferred embodiments, where a pharmaceutical composition comprises ketamine as a sublingual formulation, it may be present in an amount so that a single dose is (in a mg dose amount to be administered to the patient), e.g., between 10 mg and 100 mg inclusive, including 10 mg, 20 mg, 30 mg, 40 mg, 50 mg, 60 mg, 70 mg, 80 mg, 90 mg, and 100 mg.

In embodiments where a pharmaceutical composition comprises ketamine in a liquid solution, a dosage amount may be determined based on the concentration of ketamine in the solution; for example, where a solution of 500 mg ketamine in 5 mL liquid is used for injection, a volume of liquid for IM injection may be, e.g., 0.1 mL or less, at least 0.1 mL, at least 0.2 mL, at least 0.3 mL, at least 0.4 mL, at least 0.5 mL, at least 0.6 mL, at least 0.7 mL, at least 0.8 mL, at least 0.9 mL, or at least 1.0 mL, as well as amounts within these ranges. In embodiments where a pharmaceutical composition comprises ketamine in a liquid solution, and a solution of 500 mg ketamine in 10 mL liquid is used for injection, a volume of liquid for IM injection may be, e.g., 0.2 mL or less, at least 0.2 mL, at least 0.4 mL, at least 0.6 mL, at least 0.8 mL, at least 1.0 mL, at least 1.2 mL, at least 1.4 mL, at least 1.6 mL, at least 1.8 mL, or at least 2.0 mL, as well as amounts within these ranges.

In embodiments, a psycholytic dose of ketamine is administered. In embodiments, where a pharmaceutical composition comprises a psycholytic dose of ketamine, it may be present in an amount so that a single dose is (in a mg dose amount calculated based on the kilogram weight of the patient), e.g., 0.1 mg/kg or less, at least 0.2 mg/kg, at least 0.3 mg/kg, at least 0.4 mg/kg, at least 0.5 mg/kg, up to and including 0.6 mg/kg, as well as amounts within these ranges. In embodiments where a pharmaceutical composition comprises a psycholytic dose of ketamine, it may be present in an amount so that a single dose is (in a mg dose amount to be administered to the patient), e.g., 1 mg or less, at least 1 mg, at least 10 mg, at least 15 mg, at least 20 mg, at least 25 mg, at least 30 mg, at least 35 mg, up to and including 40 mg, as well as amounts within these ranges.

In embodiments where a pharmaceutical composition comprises a psycholytic dose of ketamine in a liquid solution, a dosage amount may be determined based on the concentration of ketamine in the solution; for example, where a solution of 500 mg ketamine in 5 mL liquid is used for injection, a volume of liquid for intramuscular injection may be, e.g., 0.1 mL or less, at least 0.1 mL, at least 0.2 mL, at least 0.3 mL, up to and including 0.4 mL, as well as amounts within these ranges. In embodiments where a pharmaceutical composition comprises a psycholytic dose of ketamine in a liquid solution, and a solution of 500 mg ketamine in 10 mL liquid is used for injection, a volume of liquid for IM injection may be, e.g., 0.2 mL or less, at least 0.2 mL, at least 0.4 mL, at least 0.6 mL, up to and including 0.8 mL, as well as amounts within these ranges.

In embodiments, a dissociative dose of ketamine is administered. In embodiments, where a pharmaceutical composition comprises a dissociative dose of ketamine, it may be present in an amount so that a single dose is (in a mg dose amount calculated based on the kg weight of the patient), e.g., at least 0.7 mg/kg, at least 0.9 mg/kg, at least 1.0 mg/kg, at least 1.1 mg/kg, at least 1.2 mg/kg, at least 1.3 mg/kg, at least 1.4 mg/kg, at least 1.5 mg/kg, as well as amounts within these ranges. In embodiments, where a pharmaceutical composition comprises a dissociative dose of ketamine, it may be present in an amount so that a single dose is (in a mg dose amount to be administered to the patient), e.g., at least 40 mg, including 45 mg, 50 mg, 55 mg, 60 mg, 65 mg, 70 mg, 75 mg, 80 mg, 85 mg, 90 mg, 95 mg, 100 mg, and doses above 100 mg, including 110 mg, 120 mg, 130 mg, 150 mg, 160 mg, 170 mg, 180 mg, 190 mg, and 200 mg, as well as amounts greater than 200 mg, including 250 mg, 300 mg, 350 mg, 400 mg, 450 mg, 500 mg, 550 mg, 600 mg, and amounts within these ranges.

In embodiments where a pharmaceutical composition comprises a dissociative dose of ketamine in a liquid solution, a dosage amount may be determined based on the concentration of ketamine in the solution; for example, where a solution of 500 mg ketamine in 5 mL liquid is used for injection, a volume of liquid for intramuscular injection may be, e.g., at least 0.5 mL, at least 0.6 mL, at least 0.7 mL, at least 0.8 mL, at least 0.9 mL, or at least 1.0 mL, as well as amounts within these ranges. In embodiments where a pharmaceutical composition comprises a dissociative dose of ketamine in a liquid solution, and a solution of 500 mg ketamine in 10 mL liquid is used for injection, a volume of liquid for intramuscular injection may be, e.g., at least 0.9 mL, at least 1.0 mL, at least 1.2 mL, at least 1.4 mL, at least 1.6 mL, at least 1.8 mL, or at least 2.0 mL, as well as amounts within these ranges.

In some embodiments, a patient receiving KAP will be administered a psycholytic dose of ketamine. In some embodiments, a patient receiving KAP will be administered a dissociative dose of ketamine. In some embodiments, a patient undergoing a KAP treatment regimen will be administered a psycholytic dose of ketamine. In some embodiments, a patient undergoing a KAP treatment regimen will be administered a dissociative dose of ketamine. It will be readily appreciated that “psycholytic” and “dissociative” dose amounts are for reference only and may differ based on individual patient characteristics, route of administration, and as otherwise described herein and understood to those of ordinary skill.

In general, it will be appreciated that a single dose of ketamine (in a mg dose amount to be administered to the patient), may be e.g., 1 mg or less, at least 1 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, or at least 200 mg (including at least 250 mg, at least 300 mg, at least 350 mg, at least 400 mg, at least 450 mg, at least 500 mg, at least 550 mg, and at least 600 mg), as well as amounts within these ranges. In some embodiments, a single dose is (in a mg dose amount to be administered to the patient), e.g., between about 50 mg to about 150 mg, including 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, and 150 mg.

It will be readily appreciated that the bioavailability of ketamine may differ depending on the method of administration utilised, but will be known or ascertainable to those of skill, and therefore dose and dosage may be modified according to that bioavailability and ordinary skill. For example, the relative bioavailability of oral ketamine is said to be between about 17% and about 24%, intramuscular ketamine is about 93%, intranasal ketamine is about 50%, rectal ketamine is about 30%, sublingual ketamine is about 30%, and nasal ketamine is about 45% (see, e.g., U.S. Pat. App. No. 2022/0125742A1, incorporated herein by reference).

It will be readily appreciated that dose and dosage may vary depending upon the treatment protocol itself, the onset, progression, severity, frequency, duration, probability of, or susceptibility of the symptom to which treatment is directed, clinical endpoint desired, previous, simultaneous or subsequent treatments, general health, age, gender, and race of the patient, bioavailability, potential adverse systemic, regional, or local side effects, the presence of other disorders or diseases in the patient, and other factors that will be appreciated by those in the art (e.g., medical or familial history).

Dose amount, frequency, or duration may be increased or reduced, as indicated by the clinical outcome desired, status of the disorder or symptoms, any adverse side effects of the treatment or therapy, or concomitant medications. Those in the art will appreciate the factors that may influence the dosage, frequency, and timing required to provide an amount sufficient or effective for providing a therapeutic effect or benefit, and to do so depending on the type of therapeutic effect desired and to avoid or minimize adverse effects.

It will be understood that, in embodiments, the dose actually administered will be determined by a physician, in light of the relevant circumstances, including the disorder to be treated, the chosen route of administration, the actual composition administered, the age, weight, and response of the individual patient, and the severity of the patient's symptoms, and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, the pharmaceutical compositions may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Dosage levels thus may differ from patient to patient, for individual patients across time, and for different pharmaceutical compositions and formulations, but shall be able to be determined with ordinary skill.

Determination of appropriate dosing shall include not only the determination of single dosage amounts, but also the determination of the number and timing of doses, and the time(s) of day or time(s) during a psychotherapeutic session preferable for administration.

In general, all of the compositions and disclosed methods will be appreciated to work for all individuals, although individual variation is to be expected, and will be readily appreciated. Where there is variation between individuals, modification to the compositions and methods will be understood based on the teachings herein in combination with general knowledge in the art. In some instances, certain personalised approaches (i.e., “personalised” or “precision” medicine) may be utilised, based on individual characteristics, including drug metabolism or individual genetic variation. The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations.

In embodiments, the genetic variation is a genetic variation in dopamine receptor D3 (DRD3) or Calcium/Calmodulin Dependent Protein Kinase II Delta (CAMK2D), which have been implicated in the pathogenesis of gambling disorder (Lobo et al., 2015). In embodiments, the genetic variation is a genetic variation in metabotropic glutamate receptor type 5 (mGluR5), which has been implicated in mood and anxiety symptoms. In embodiments, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders (Yehuda 2016, Bierer 2020). In embodiments, the genetic variation is a genetic variation such as a SNP in a membrane transporter, such as SERT, DAT, NET, or VMAT. In embodiments, the patient being treated has altered epigenetic regulation of a gene the expression of which is associated with a mental health condition or susceptibility to a mental health treatment, such as the SIGMAR1 gene for the non-opioid sigma-1 receptor.

In embodiments, the genetic variation is a genetic variation in one or more cytochrome P450 (CYP or CYP450) enzymes that affects drug metabolism, including metabolism of a composition of the invention. In embodiments, the genetic variation may be in any of CYP1A2, CYP2C9, CYP2D6, CYP2C19, CYP3A4, CYP3A5, CYP1A1, CYP1B1, CYP2A6, CYP2A13, CYP2B6, CYP2C8, CYP2C9, CYP2C18, CYP2E1, CYP2G1, CYP2J2, CYP2R1, CYP2S1, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In embodiments, a composition of the invention is taken together with a compound that is metabolised by the same CYP enzyme(s) as the ketamine compound(s) administered (e.g., CYP2C9, CYP2B6, or CYP3A4), so as to permit a lower dose to be taken, increase the effective bioavailability of one or both, or otherwise affect drug metabolism or pharmacokinetics. In embodiments, the dose of a composition is adjusted when administered to a patient known to be a “poor metabolizer” of an active agent in the composition.

In embodiments, a genetic variation (or a genetic marker signifying a variation) may instruct a physician to administer a lesser dose of the composition. In embodiments, a genetic variation may instruct a physician to administer a greater dose of the composition.

In embodiments, especially where a formulation is prepared in single unit dosage form, suggested dosage amounts shall be known by reference to the format of the preparation itself. In other embodiments, suggested dosage amounts may be known by reference to the means of administration or by reference to the packaging and labeling, package insert(s), marketing materials, training materials, or other information and knowledge available to those of skill or the public. Another aspect of this disclosure provides pharmaceutical kits comprising a ketamine composition, suggested administration guidelines or prescribing information therefore, and a suitable container. Individual unit dosage forms can be included in multi-dose kits or containers. Pharmaceutical compositions also can be packaged in single or multiple unit dosage forms for uniformity of dosage and ease of administration.

### D. Treatment of Behavioural Addictions

In some aspects are methods of treating a patient with a behavioural addiction. Herein, “treating a patient” with a behavioural addiction or a disorder will be used interchangeably with terms like “treating a behavioural addiction” and “treating a disorder”; terms like “treatment of a patient” and “treatment of a disorder” are likewise used similarly.

Herein, “treating” or “treatment” of a disorder includes any treatment of the disorder in a mammal, and preferably in a human, and includes: (a) preventing a disorder from occurring in a patient who may be predisposed to the disorder but has not yet been diagnosed with it; (b) inhibiting a disorder, i.e., arresting its development, and including prophylaxis; (c) relieving a disorder, i.e., causing regression of the disorder or its clinical symptoms; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder or comorbid with a disorder. Other such measurements, benefits, and surrogate or clinical endpoints, alone or in combination, would be understood to one of skill based on the teachings herein and general knowledge in the art.

In embodiments, the patient with a behavioural addiction is administered ketamine. In embodiments, the administration of ketamine is effective to treat a behavioural addiction. In embodiments, the administration of ketamine is effective to treat a patient with a behavioural addiction, including a patient diagnosed with a behavioural addiction. In embodiments, treatment may be evident by a decrease of one or more points of a patient completed self-assessment, as described herein, and/or a decrease of one or more points in a clinical outcome assessment or index score, as compared to baseline values, as disclosed herein.

In embodiments, a patient with a behavioural addiction is administered ketamine together with psychotherapy, wherein the psychotherapy uses one or more psychotherapeutic techniques as defined herein. In embodiments, the administration of ketamine together with psychotherapy is effective to treat the patient with the behavioural addiction.

Herein, “psychotherapeutic techniques” include, in embodiments, any form of “psychosocial or behavioural therapy” which refers to, but is not limited to, 12-step facilitation therapy (e.g., NIAAA, Project MATCH Monograph Series. Volume 1, NIH Publication No. 94-3722, (1995) reprinted 1999), cognitive behavioural therapy (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2): 354-59; or in Case Reports in Psychiatry, Vol. 2012, Article ID 731638), community reinforcement approach based therapy (e.g., as described in Drug Alcohol Depend 2004; 74:1-13), motivational interviewing based therapy (e.g., as described in J. Consul. Clin. Psychol. 2001; 69(5): 858-62), motivational enhancement based therapy (e.g., as described in Drug Alcohol Depend 2007, 91:97-101) or meditation based therapy, such as transcendental meditation based therapy (e.g., as described in Addiction 2004; 99(7):862-874 or J. Consul. Clin. Psychol. 2000; 68(3): 515-52); Acceptance and Commitment Therapy (Hayes et al., 2011; Watts & Lumoa, 2020; Sloshower et al., 2020).

Additional psychotherapeutic techniques useful in the practice of the disclosed ketamine-assisted psychotherapy methods include as further examples, dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, and mindfulness-based cognitive therapy.

Psychotherapeutic techniques, in some embodiments, comprise psychological support. In some embodiment, instead of psychotherapy, a patient is monitored. Monitoring may take place in person or virtually. Monitoring may be by one or more therapists, and/or also may be through a software and/or hardware device or system, capable of monitoring a patient.

“standardised psychotherapy” or “standardised psychological support” refers to standardised sessions, which may be, e.g., once a week, twice a week, once every two weeks, once a month, and the like, of any psychotherapeutic technique or of psychological support used in the treatment of a behavioural addiction patient according to the disclosed methods.

In embodiments, any of the disclosed methods may be used in combination with one or more therapeutically beneficial activities, where such participation follows or is together with the administration of a ketamine composition, including breathing exercises, meditation and concentration practices, cold therapy, focusing on an object or mantra, listening to music, physical exercise, stretching or bodywork, yoga, journaling, grounding techniques, positive self-talk, or engaging with a pet or animal, and it should be understood that such participation can occur with or without the participation or guidance of a therapist or other professional.

In some embodiments, a patient will be prescribed a ketamine composition of the invention for use at home, and the ketamine composition may be mailed to the patient's home. In some embodiments, the patient will receive KAP or otherwise undergo psychotherapy via telemedicine or other remote means. In some embodiments, the patient will self-administer the ketamine composition outside the presence of a therapist, both in person and virtually, although the patient may be otherwise under the care of a physician, and may be undergoing a separate course of psychotherapy involving non-drug-assisted sessions. In embodiments, the patient may be instructed on the self-administration of ketamine using a smartphone app, computer program, web-based software, or similar means. In embodiments, the patient instructions on the self-administration of ketamine also include pre-recorded information from a therapist, or may include guided imagery, a guided meditation, or the like.

Herein, “therapist” refers to a person who treats a patient using the disclosed compositions and methods, such as by administering ketamine together with psychotherapy (i.e., “ketamine-assisted psychotherapy,” “KAP,” “ketamine-assisted therapy,” or “KAT,” all used interchangeably herein), whether the person is a psychiatrist, clinical psychologist, clinical therapist, registered therapist, psychotherapist, or other trained clinician, counselor, facilitator, or guide, although it will be understood that certain requirements are appropriate to certain aspects of treatment (e.g., prescribing, dispensing, or administering ketamine or another drug, offering psychotherapeutic support, diagnosing a symptom or disorder, etc.).

In embodiments, measures of therapeutic effect may be utilised to determine the effectiveness of treatment. In embodiments, such measures of therapeutic effect include any outcome measure, endpoint, effect measure, assessment, or measure of effect within clinical or medical practice or research which is used to assess the effect, both positive and negative, of an intervention or treatment, whether patient-reported, gathered through laboratory tests such as blood work, urine samples etc., or through medical examination. Measures of therapeutic effect for purposes of the invention will be understood to include, without limitation: (1) frequency of the compulsive or problematic behaviour, (2) drive or urge to engage in the compulsive or problematic behaviour, (3) abstinence from the compulsive or problematic behaviour, (4) relapse into the compulsive or problematic behaviour, (5) severity of symptoms of the compulsive or problematic behaviour, (6) time spent on the compulsive or problematic behaviour; (7) quality of life, including subjective sleep; and (8) psychosocial functioning. Those of skill will appreciate that other equivalent or similar measures of therapeutic effect also may be used, and others are described herein and claimed.

In embodiments, patients who receive treatment according to the disclosed methods will also be assessed to determine: (1) prescribed medication use; (2) number and frequency of recreational drugs used (if any); and (3) cravings relating to ketamine; and (4) any subsequent use of illicit ketamine. In embodiments, treatment according to the disclosed methods will result in no change to any one or more of, will result in a reduction in one or more of, will result in an elimination of one or more of, will result in no initiation of any one or more of, or will result in no increase in any one of more of: (1) prescribed medication use; (2) number and frequency of recreational drugs used (if any); and (3) cravings relating to ketamine; and (4) any subsequent use of illicit ketamine.

In embodiments wherein assessments, outcome measures, and/or measures of therapeutic effect, including but not limited to those disclosed in TABLE 2, are utilised to assess the effectiveness of prolonged effectiveness of treating according to the disclosed methods, such assessments may be completed after a period of months after treatment, such as at least 1 day, at least 1 week, at least 1 month, or at least 1 year from baseline.

Herein, “assessment” refers to a means or method used with a patient to measure, estimate, or evaluate a disorder, symptom, or characteristic of the patient, whether qualitatively or quantitatively, and whether performed by the therapist or other clinician (e.g., an interview), by the patient his or herself (e.g., a self-reported questionnaire), by a third-party or by a computer, including a medical device (e.g., as defined by FDA or another regulatory body) or other device (e.g., a medical sensor or biosensor, a watch or fitness tracker), and whether graded by a human decision-maker or an artificial intelligence, machine learning, or computer algorithm. Assessments may include self-report measures, patient-reported surveys, questionnaires, interviews, and the like. Non-limiting examples of assessments, useful in the disclosed methods, include those in TABLE 2 below.

TABLE 2
Exemplary Patient Assessments

| | |
|---|---|
| 1 | Gambling Attitude and Beliefs 23 Survey (GABS-23) (Bouju et al., 2014). A |
| | 23-question survey that explores gambling-related cognitions through five |
| | dimensions. All items are coded on a scale ranging from strongly disagree = 0 to |
| | strongly agree = 3. The raw subscale scores are obtained by adding the scores of the |
| | items composing each subscale and range from 0 to 12 or 15. |
| 2 | The Mini International Neuropsychiatric Interview 5 (MINI 5) (Sheehan et al. |
| | 1998) is used to screen for comorbid psychiatric conditions. |
| 3 | The Columbia Suicide Severity Rating Scale (C-SSRS) (Mundt, J C et al. 2013), to |
| | screen for acute and recent suicide and self-harm thoughts and behaviours, taking |
| | approximately five minutes to complete. |
| 4 | The Patient Health Questionnaire (PHQ-9) (Kroenke et al. 2001). A brief |
| | self-administered screening questionnaire for depressive symptoms. |
| 5 | Generalised Anxiety Disorder 7 (GAD-7) (Spitzer et al. 2006) is a |
| | self-reported questionnaire for screening and severity measuring of generalised |
| | anxiety disorder (GAD). |
| 6 | The Gambling Timeline Follow Back (G-TLFB) (Sobell 2001). A measure of |
| | gambling behaviour using the timeline followback methodology to assess the intensity |
| | and frequency of gambling behaviour over time. Although not initially designed to |
| | assess gambling, the TLFB method has been used to assess gambling behaviour in |
| | several treatment studies, and use in these studies has provided support that a specific |
| | treatment leads to positive changes in gambling behaviour (Hodgins et al., 2001). |
| 7 | Problem Gambling Severity Index (PGSI) (Ferris & Wynne, 2001). A nine-item |
| | screening tool that can be self-administered or administered by a clinician. A briefer |
| | version of the 31-item Canadian Problem Gambling Index, using a four-point scale |
| | (“never” to “almost always”) to describe gambling behaviour over the last 12 months. |
| 8 | South Oaks Gambling Screen (SOGS) (Lesieur and Bloom, 1990). This is a 20-item |
| | scale shown to accurately identify clients with problem gambling. It was developed |
| | using DSM-III criteria so it does not reflect the DSM-5 criteria. It has also been |
| | revised and validated for use with adolescents. |
| 9 | The “Gambling Attitudes Scale” (GAS) (Kassinove 1998) assesses attitudes |
| | (affective, cognitive and behaviour-related aspects) concerning gambling in general |
| | and specifically in casinos, horse betting and lottery, which can foster the |
| | development of pathological gambling. |
| 10 | Pittsburgh Sleep Quality Index (PSQI) (Buysse 1989) is used to assess the level of |
| | sleep disturbance. |
| 11 | Interpersonal reactivity Index (IRI) (Davis 1980) comprises 28 items answered on a |
| | 5 point scale. This scale measures different aspects of empathy and provides different |
| | subscales relating to these. |
| 12 | The Short Form (36) Health Survey (SF-36) is a gold standard patient-reported |
| | measure of quality of life. |
| 13 | The Self-Compassion Scale (SCS) (Neff 2003) Comprises 26 items answered on a 5 |
| | point scale. This scale measures core aspects of self-compassion including |
| | components of mindfulness. |
| 14 | The Trauma History Questionnaire (THQ) (Green 1996) is a self-report measure |
| | that examines experiences with potentially traumatic events using a yes/no format. For |
| | each event endorsed, respondents are asked to provide the frequency of the event as |
| | well as their age at the time of the event. |
| 15 | The PTSD Checklist for DSM-5 (PCL-5) The PCL-5 is a 20-item self-report |
| | measure that assesses the 20 DSM-5 symptoms of PTSD. |
| 16 | Adjustment Disorder - New Module 20 (ADNM-20 Questionnaire) a 20-item |
| | questionnaire for adjustment disorder. |
| 17 | Alcohol, Smoking, and Substance Involvement Screening Test (ASSIST) An |
| | 8-item screening tool developed for the World Health Organization (WHO) by an |
| | international group of substance abuse researchers to detect and manage substance use |
| | and related problems in primary and general medical care settings. |
| 18 | NIDA Drug Use Screening Tool A 1- to 7-question screening tool adapted from the |
| | WHO's ASSIST by the National Institute on Drug Abuse (NIDA). |
| 19 | Yale-Brown Obsessive-Compulsive Scale (Y-BOCS) (Goodman et al., 1989). The |
| | most widely-used tool for assessing global severity of OCD, containing two parts: a |
| | checklist of over 50 types of obsessions and compulsions, and a 10-item severity scale |
| | for rating the most prominent obsessions and compulsions (Eilersten et al., 2017). |
| 20 | The Internet Gaming Disorder Test (IGD-20 Test) (Pontes et al., 2014) was the first |
| | standardised psychometric tool to assess Internet Gaming Disorder (IGD) according |
| | according to the nine Internet Gaming Disorder (IGD) criteria as suggested by the |
| | American Psychiatric Association (APA) in the DSM-5. |
| 21 | Internet Gaming Disorder Scale-Short-Form (IGDS9-SF) (Pontes et al., 2015) |
| | was the first brief standardised psychometric tool to assess Internet Gaming Disorder |
| | (IGD) according according to the nine Internet Gaming Disorder (IGD) criteria as |
| | suggested by the American Psychiatric Association in the DSM-5. |
| 22 | Compulsive Sexual behaviour Inventory (CSBI-13) a 13-item, five point Likert |
| | scale, may be utilised to determine the presence of compulsive sexual behaivour |
| | disorder, with a score above a 35 indicating the presence of CSBD (Coleman, n.d.). |
| 23 | YBOCS-Shopping Scale (YBOCS-SV), was modeled on the Y-BOCS (Goodman et |
| | al., 1989) and assesses severity and interference caused by buying thoughts and |
| | behaviour rather than of assessing obsessions and compulsions. |
| 24 | Digital Addiction Scale (DAS), which measures addiction on a scale of 1-5, 5 being |
| | the highest addiction level (Kesici & Tunc, 2018) |
| 25 | Kleptomania Symptom Assessment Scale (K-SAS), is an 11-item self-report scale |
| | designed to measure symptoms of kleptomania. Each item is scored on a scale from 0 |
| | to 4 (Asami et al., 2020) |
| 26 | The Clinical Global Impressions Scale (CGI), measures the severity of |
| | psychopathology from 1 to 7 and change from the initiation of treatment on a similar |
| | seven-point scale (Busner and Targum, 2007). |
| 27 | The Sheehan Disability Scale (SDS), The SDS is a five-item, self-rated |
| | questionnaire designed to measure the extent to which a patient's disability due to an |
| | illness or health problem interferes with work/school, social life/leisure activities, and |
| | family life/home responsibilities (Coles et al., 2014). |
| 28 | The Global Assessment of Functioning Scale (GAF), is used to rate how serious a |
| | mental illness may be by measuring how much a person's symptoms affect their |
| | day-to-day life on a scale of 0-100 (Smith, 2021). |
| 29 | The Fire Setting Scale (FSS), is a 20-item scale containing two 10-item subscales |
| | developed to measure antisocial behavioural problems relating to firesetting and |
| | general fire interest (Gannon and Barrowcliffe, 2010). |
| 30 | The Fire Proclivity Scale (FPS), measures the propensity of an individual to engage |
| | in firesetting (Gannon and Barrowcliffe, 2010). |
| 31 | St. Andrews Fire and Risk Instrument (SAFARI), is semi-structured interview |
| | assessment specifically for fire-setting to augment structured assessments of risk and |
| | need (Long et al., 2014). |
| 32 | Internet Addiction Test (IAT), a 20 item evaluation of attributes and demeanors |
| | related to obsessive use of the internet that comprises escapism, compulsivity, and |
| | dependency (Samaha et al., 2018). |
| 33 | Chen's Internet Addiction Scale (CIAS), a 4-point, 26-item self-report scale |
| | assessing five dimensions of Internet addiction: compulsive use, withdrawal, |
| | tolerance, problems with interpersonal relationships, and problems with health and |
| | time management (Hung Ko et al., 2019). |
| 34 | Brief Pornography Screener (BPS), is a five-item self-report questionnaire |
| | developed to detect problematic use of pornography among clinical and non-clinical |
| | samples (Kraus and Grubbs, 2019). |
| 35 | Problematic Pornography Consumption Scale (PPCS-6), a six-component |
| | addiction model that can distinguish between non-problematic and problematic |
| | pornography use (Bothe et al., 2017). |
| 36 | Binge Eating Scale (BES), a 16-item questionnaire assessing the presence of certain |
| | binge eating behaviours which may be indicative of an eating disorder |
| | (Escriva-Martinez et al., 2019). |
| 37 | Lie-Bet Tool to Rule Out Problem Gambling, is a two-item tool used to rule out |
| | pathological gambling behaviours. |
| 38 | National Opinion Research Center DSM Screen for Gambling Problems (NODS), |
| | is a population-based telephone-screening tool to identify gambling problems using |
| | DSM-IV criteria, and an outcome measure for gambling studies (Hodgins, 2004). |
| 39 | Loss of Control, Lying, and Preoccupation screen (NODS-Clip), an efficient |
| | standardised diagnostic interview instrument for adult pathological and problem |
| | gambling (Toce-Gerstein, Gerstein, and Volberg, 2009). |
| 40 | Sensitivity to Punishment and Sensitivity to Reward Questionnaire (SPSRQ), is a |
| | self-reported instrument that includes 48 yes/no questions divided into two subscales: |
| | sensitivity to reward (SR) and sensitivity to punishment (SP) (Torrubia et al., 2001). |
| 41 | Gambling Related Cognitions Scale (GRCS), is a 23-item scale used to identify |
| | gambling-related conditions in gamblers (Raylu and Oei, 2004). |
| 42 | Beck Depression Inventory (BDI), useful in determining whether a patient has |
| | depression and, if so, the severity of depression (Beck, Steer, and Brown, 1996). |
| 43 | Adult Attachment Interview, a 20 question interview inquiring about subject's |
| | recollections of their own childhood (Sykes and Turner, 2015). |
| 44 | Morningness-Eveningness Questionnaire (MEQ). A 19-item questionnaire to |
| | assess when a patient prefers to wake up and start sleep (Am. Thoracic Society, 2022). |
| 45 | Social Phobia Inventory (SPIN), is a 3-item, self-rated scale originally developed to |
| | screen for generalised social anxiety disorder (Cuncic, 2021). |

In embodiments, an assessment may or may not be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps, and also including methods of digital phenotyping. Computer-assisted assessments will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like).

One or more other aspects of KAP additionally may be computer-assisted, for example where one or more steps of treatment involve the use of a computer to complement, in addition to, or as a replacement for work which would otherwise be performed by a therapist, and KAP further may include the use of virtual reality (VR), augmented reality (AR), mixed reality, and other immersive or semi-immersive devices and systems.

### Example 7: Ketamine in Subjects with Gambling Disorder

A study was completed on the use of ketamine to treat gambling disorder. Subjects were assessed for gambling disorder using the PGSI. Subjects were included if they received a PGSI score greater than 3. Five subjects in total were included (G1-G5). All five subjects were administered ketamine. Blood samples were collected from all five subjects. Blood samples were analysed to determine ketamine and ketamine metabolite levels, and each subject was assessed post-administration to determine the effectiveness of the treatment.

### Methods

The study spanned a total of 30 days (not counting initial contact and prescreening). Prescreening of all subjects was conducted by phone, and subjects recorded self-report measures, including the Sensitivity to Punishment and Sensitivity to Reward Questionnaire (SPSRQ) and the Gambling Related Cognitions Questionnaire. Prior to beginning treatment, each subject completed the Beck Depression Inventory (BDI) to determine a baseline depression score. Total scores of 0-13 are minimal, 14-19 are mild, 20-28 are moderate, and 29-63 are severe (Beck, Steer, and Brown, 1996).

Screening and baseline measures were collected onsite on Day 1, when the above self-report measures were conducted again. Behavioural tasks were completed, including: a cue reactivity task in which each subject was shown various images of poker, bingo, scratch cards, and neutral photographs before being asked (1) how pleasant the picture is to look at, (2) how much does this picture make you want to gamble, and (3) to create a picture pleasure rating and a gambling desire rating for each photo; a risk taking task; and a delay discount task, in which preference for small immediate rewards over larger delayed rewards was assessed, where subjects calculate the value of each reward. EEG measurements were collected. These measures were used to determine a baseline for each subject.

On Day 3, each subject returned on-site and was administered ketamine. 100 mg of racemic ketamine was administered in as sublingual lozenges.

Blood samples were collected every 10 minutes, from 0 minutes (the time of administration) to 90 minutes post-administration. Blood samples at each time point were analysed to determine plasma levels of (2S,6S;2R,6R)-Hydroxynorketamine (HNK).

Reward sensitivity was assessed using the SPSRQ. Behavioural tasks, EEG measurements, and subjective experience assessments were also completed.

The remaining follow-up sessions were completed online. Follow-up 1 was completed on Day 4, Follow-up 2 on Day 14, and Follow-up 3 on Day 30. At each follow-up session, the same self-report measures and behavioural tasks were completed.

### Results and Discussion

### Overall Safety and Tolerability

Adverse events were assessed with the Ketamine Side Effect Tool (KSET). No unexpected adverse reactions were observed. All adverse events were transient, and resolved two hours post-lozenge. Overall, the study was well-tolerated, with no drop-outs.

### Ketamine Levels

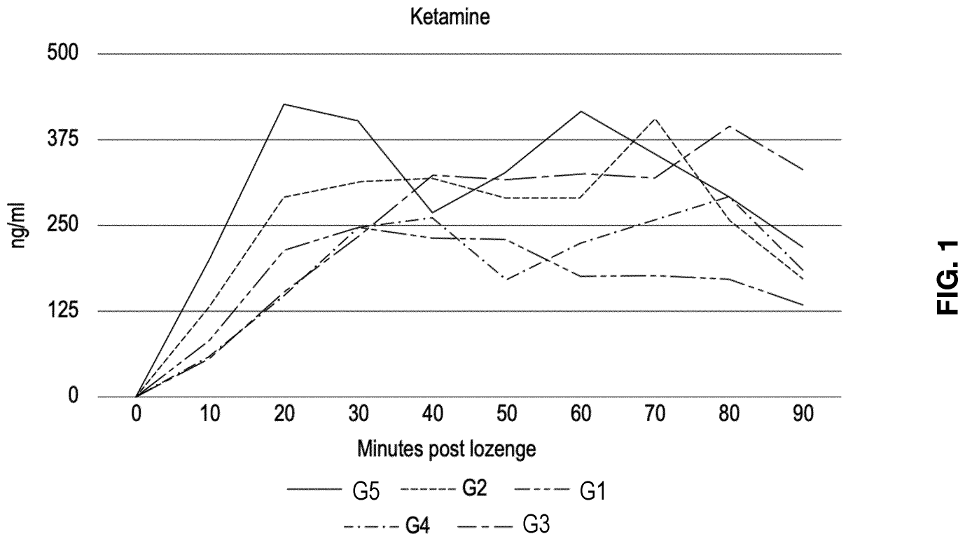

FIG. 1 illustrates that subject G1 had a peak concentration of about 250 ng/ml at 30 minutes post-administration; G2 had a peak concentration of about 405 ng/ml at 70 minutes post-administration; G3 had a peak concentration of about 395 ng/ml at 80 minutes post-administration; G4 had a peak concentration of about 291 ng/ml at 80 minutes post-administration; and G5 had a peak concentration of about 426 ng/ml at about 20 minutes post-administration.

### Ketamine Metabolite Levels

(2S,6S;2R,6R)-Hydroxynorketamine (HNK) and Norketamine were each assessed.

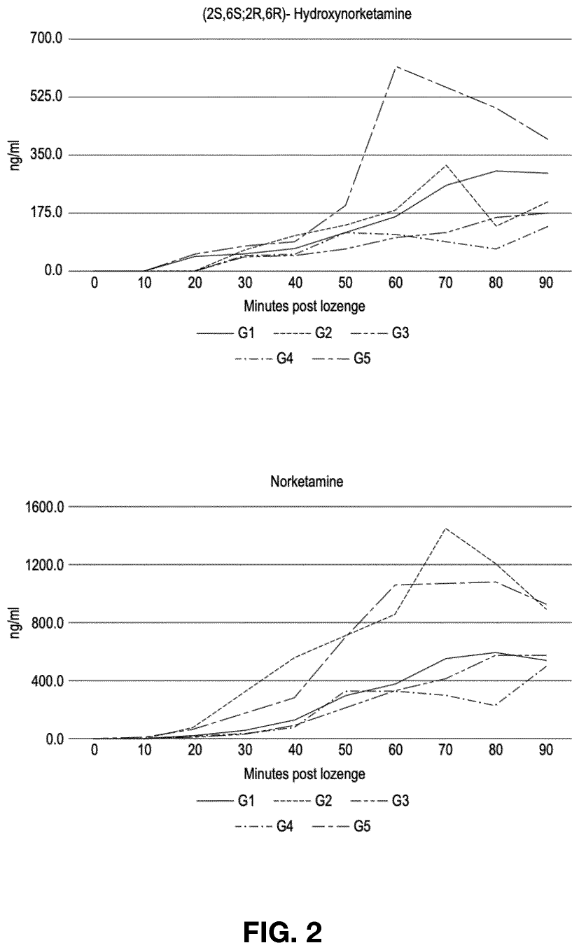

HNK: FIG. 2 illustrates that subject G1 had a peak concentration of about 300 ng/ml at 80 minutes post-administration; G2 had a peak concentration of about 319 ng/ml at 70 minutes post-administration; G3 had a peak concentration of about 175 ng/ml at 90 minutes post-administration; G4 had a peak concentration of about 134 ng/ml at 90 minutes post-administration; and G5 had a peak concentration of about 615 ng/ml at about 60 minutes post-administration.

Norketamine: FIG. 2 illustrates that subject G1 had a peak concentration of about 592 ng/ml at 80 minutes post-administration; G2 had a peak concentration of about 1,450 ng/ml at 70 minutes post-administration; G3 had a peak concentration of about 574 ng/ml at 80 minutes post-administration; G4 had a peak concentration of about 495 ng/ml at 90 minutes post-administration; and G5 had a peak concentration of about 1,079 ng/ml at about 90 minutes post-administration.

### Change in Symptoms and GCRS

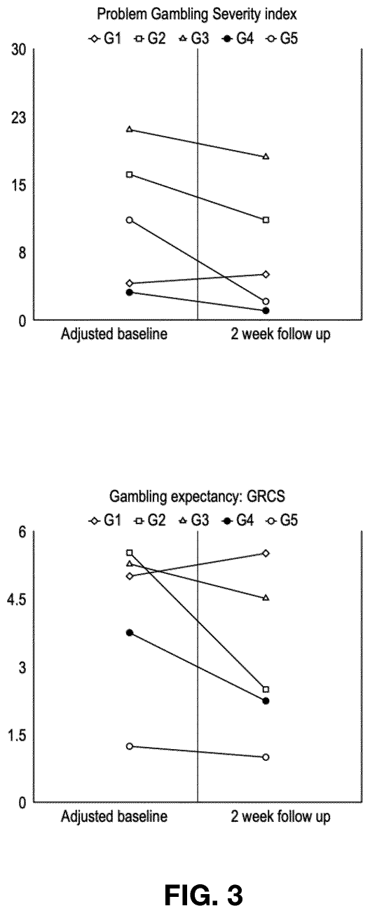

Problem Gambling Symptoms: FIG. 3 illustrates that subject G1 increased from a preadministration score of 4 to a score of 5 at the 2-week follow up, an increase of 25%; G2 decreased from a pre-administration score of 16 to a score of 11 at the 2-week follow up (31.25%); G3 decreased from a pre-administration score of 21 to a score of 18 at the 2-week follow up (14.29%); G4 decreased from a pre-administration score of 3 to a score of 1 at the 2-week follow up (66.67%); and G5 decreased from a pre-administration score of 11 to a score of 2 at the 2-week follow up (81.82%); see also TABLE 3:

TABLE 3
Change in PGSI Score from Pre-Administration to 2-Week Follow Up
Change in Problem Gambling

| Subject | G1 | G2 | G3 | G4 | G5 |
|---|---|---|---|---|---|
| Pre- | 4 | 16 | 21 | 3 | 11 |
| administration | | | | | |
| Score | | | | | |
| Score at 2-week | 5 | 11 | 18 | 1 | 2 |
| follow up | | | | | |
| % change | +25% | −31.25% | −14.29% | −66.67% | −81.82% |

The PGSI classifies potential problem gamblers in four categories based on scores. A score of 0 is a non-problem gambler, a score of between 1 and 2 is a low-risk gamber (mild), a score of 3-7 is a moderate-risk gambler (moderate), and a score of 8 or above is a problem gambler (severe). Subject G4 was classified as a moderate-risk (moderate) gambler pre-administration, and at the two-week follow up was classified as a low-risk (mild) gambler. Subject G5 was classified as a problem (severe) gambler pre-administration, and at the two-week follow up was classified as a low-risk (mild) gambler

The five-factor Gambling Related Cognitions Scale (GRCS) was also assessed. The GRCS evaluates gambling-related cognitive distortions for all types of gamblers, regardless of their gambling activities (poker, slots, horse racing, etc.) (Levesque et al., 2017). The GRCS contains five main domains, including inability to stop, expectancies, predictive control, illusion of control, and interpretative bias (Muela, Navas, and Perales, 2021). Gambling expectancy and inability to stop were predictive of problem gambling.

Problem Gambling Expectancy: As illustrated in FIG. 3, subject G1 had an increase at the 2-week follow up (5 to 5.5, a 10% increase); G2 decreased from a pre-administration score of 5.5 to a score of 2.5 at the 2-week follow up (54.55%); G3 decreased from a pre-administration score of 5.25 to a score of 4.5 at the 2-week follow up (14.29%); G4 decreased from a pre-administration score of 3.75 to a score of 2.5 at the 2-week follow up (33.33%); and G5 decreased from a pre-administration score of 1.25 to a score of 1 at the 2-week follow up (20%); see also TABLE 4:

TABLE 4
Problem Gambling Expectancy: Pre-Administration to 2-Week Follow Up
Change in Problem Gambling Expectancy

| Subject | G1 | G2 | G3 | G4 | G5 |
|---|---|---|---|---|---|
| Pre-administration | 5 | 5.5 | 5.25 | 3.75 | 1.25 |
| Score | | | | | |
| Score at 2-week | 5.5 | 2.5 | 4.5 | 2.5 | 1 |
| follow up | | | | | |
| % change | +10% | −54.55% | −14.29% | −33.33% | −20% |

FIGS. 2 and 3 illustrate that subject G2 had the highest plasma concentration of norketamine (1,450 ng/ml at 70 minutes) and had the highest reduction in problem gambling expectancy (5.5 pre-administration to 2.5 at the two-week follow up, a decrease of 54.55%). Gambling expectancy includes one's belief that gambling will lead to positive consequences (e.g., the accumulation of wealth, an increase in positive emotions, etc.).

### Picture Pleasure Rating and Gambling Desire Rating:

Picture pleasure rating decreased from a mean of 3.25 at baseline, to a mean of 1.33 at the one-week follow up, and a mean of −0.83 at the two-week follow up. Gambling desire rating decreased from a mean of 2.25 at baseline to a mean of −0.25 at the one-week follow up, and a mean of −0.25 at the two-week follow up.

### Example 8: Ketamine in Subjects with Binge Eating Disorder

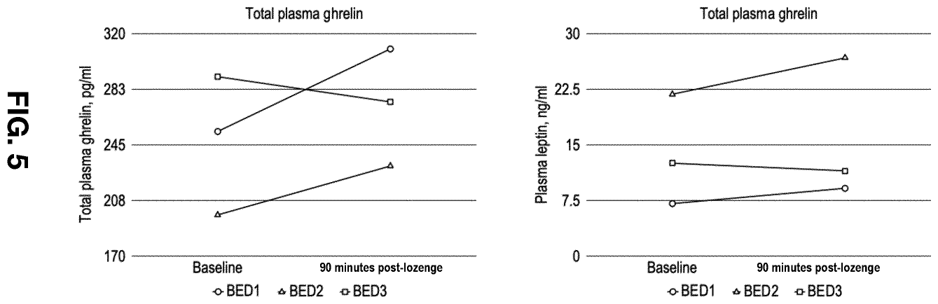

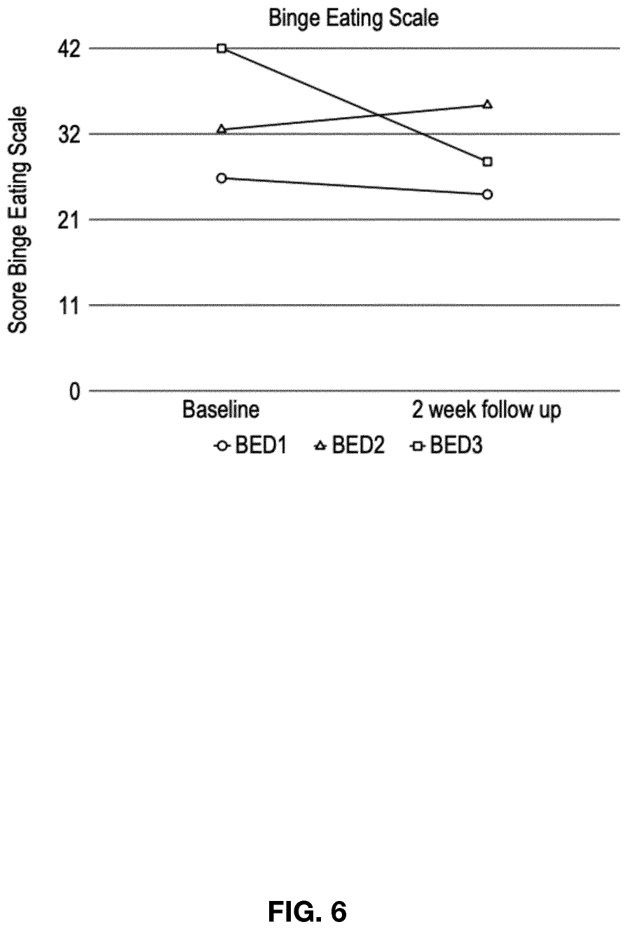

A study was completed on the use of ketamine to treat binge eating disorder. Subjects were assessed for binge eating disorder using the Binge Eating Scale (BES), and included if they received a BES score greater than 15. Three subjects in total were included (BE1-BE3, and interchangeably on FIGS. 5 and 6 as BED1-BED3, these being the same subjects). All three were administered sublingual lozenges comprising 100 mg of racemic ketamine.

Blood samples were collected from all three subjects. Blood samples were analysed to determine ketamine and ketamine metabolite levels, as well as the hormones leptin and ghrelin. Self-report measures were conducted.

### Methods

The treatment protocol commenced in substantially the same way as disclosed in the ketamine in subjects with gambling disorder study completed in EXAMPLE 7, with the exceptions outlined as follows: No behavioural tasks were completed. Blood samples collected every 10 minutes from the time of administration to 90 minutes post-administration were additionally analysed for leptin and ghrelin. Self-report measures included the Binge Eating Scale, the Adult Attachment Interview (AAI), a 20 question interview about the subject's recollections of their childhood (Sykes and Turner, 2015); the Childhood Trauma Questionnaire; the Morningness-Eveningness Questionnaire (MEQ), a 19-item questionnaire designed to assess when a respondent prefers to wake up or start sleep (American Thoracic Society, 2022); the DSM-5 Binge Eating Disorder diagnostic criteria; and the SPSRQ.

### Results and Discussion

### Adult Attachment Interview

Data for the Adult Attachment Interview follows: for the close sub scale, subject BE1 scored a total of 22 with mean of 3.66; BE2 scored a total of 17 with a mean of 2.83; and BE3 scored a total of 17 with a mean of 2.83; for the depend sub scale, subject BE1 scored a total of 19 with a mean of 5.43; BE2 scored a total of 15 with a mean of 4.29; and BE3 scored a total of 19 with a mean of 5.43; for the anxiety sub scale, subject BE1 scored a total of 14 with a mean of 2.33; BE2 scored a total of 23 with a mean of 3.83; and BE3 scored a total of 19 with a mean of 3.16.

### Childhood Trauma Questionnaire

Data for the Childhood Trauma Questionnaire follows: for emotional abuse, subject BE1 scored 8; BE2 scored 11; and BE3 scored 16; for physical abuse, subject BE1 scored 5; BE2 scored 8; and BE3 scored 18; for sexual abuse, subject BE1 scored 5; BE2 scored 5; and BE3 scored 7; for emotional neglect, subject BE1 scored 8; BE2 scored 13; and BE3 scored 8; and for physical neglect, subject BE1 scored 5; BE2 scored 5; and BE3 scored 9.

### Overall Safety and Tolerability

The pattern of adverse events was assessed with KSET and no unexpected adverse reactions were observed. All adverse events were transient, and resolved two hours post-lozenge. Overall, the study was well-tolerated, with no drop-outs.

### Ketamine Levels

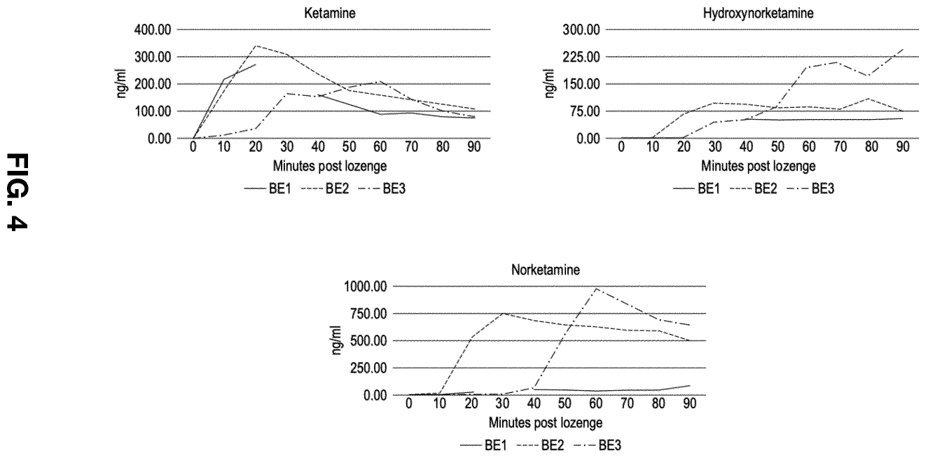

FIG. 4 illustrates that subject BE1 had a peak concentration of about 270 ng/ml at 20 minutes post-administration (no data exists for BE1 at 30 minutes); BE2 had a peak concentration of about 339 ng/ml at 20 minutes post-administration; and BE3 had a peak concentration of about 208 ng/mL at 60 minutes post-administration.

### Ketamine Metabolite Levels

(2S,6S;2R,6R)-Hydroxynorketamine (HNK) and Norketamine were each assessed.

HNK: As illustrated in FIG. 4, subject BE1 had a peak concentration of about 53 ng/ml at 90 minutes post-administration (no data exists for BE1 at 30 minutes); BE2 had a peak concentration of about 108 ng/ml at 80 minutes post-administration; and BE3 had a peak concentration of about 244 ng/mL at 90 minutes post-administration.

Norketamine: As illustrated in FIG. 4, subject BE1 had a peak concentration of about 81 ng/ml at 90 minutes post-administration (no data exists for BE1 at 30 minutes); BE2 had a peak concentration of about 685 ng/ml at 40 minutes post-administration; and BE3 had a peak concentration of about 975 ng/mL at 60 minutes post-administration.

### Change in Appetite Hormones

Changes in ghrelin and leptin were measured. Ghrelin stimulates appetite and food intake, increases fat deposition, and releases growth hormone. Obese binge eating disorder subjects often have reduced plasma ghrelin, possibly due to the downregulation of ghrelin from excess eating or body fat (Atalayer et al. 2013). Leptin forms a negative feedback loop between fat cells and the hypothalamus, wherein leptin secretion signals the hypothalamus to inhibit food intake and increase energy expenditure. Increased leptin may aid in reducing appetite cravings associated with binge eating disorder (Obradovic et al. 2021).

### Total Ghrelin

As illustrated in FIG. 5, subject BE3 had a decrease in total plasma ghrelin (291 pg/ml pre-administration to 274 pg/ml at 90 minutes post-lozenge, a 5.84% reduction); BE1 had an increase from 254 pg/ml pre-administration to 310 pg/ml at 90 minutes post-lozenge (22.05%); and BE2 had an increase from 198 pg/ml pre-administration to 231 pg/ml at 90 minutes post-lozenge (16.67%); see also TABLE 5:

TABLE 5
Change in Total Ghrelin from Pre-Administration to 2-Week Follow Up
Change in Total Ghrelin

| Subject | BE1 | BE2 | BE3 |
|---|---|---|---|
| Pre-administration (pg/ml) | 254 | 198 | 291 |
| At 90 minutes post-lozenge (pg/ml) | 310 | 231 | 274 |
| % change | +22.05% | +16.67% | −5.84% |

### Total Leptin

FIG. 5 illustrates that subject BE3 had a decrease in total plasma leptin (12.36 ng/ml pre-administration to 11.23 ng/ml at 90 minutes post-lozenge, a 9.1400 reduction); BE1 had an increase from 7.10 ng/ml pre-administration to 9.11 ng/ml at 90 minutes post-lozenge (28.310%); and BE2 had an increase from 21.88 ng/ml pre-administration to 27.98 ng/ml at 90 minutes post-lozenge (27.880); see also TABLE 6:

TABLE 6
Change in Total Leptin from Pre-Administration to the 2-Week Follow Up
Change in Total Leptin

| Subject | BE1 | BE2 | BE3 |
|---|---|---|---|
| Pre-administration (ng/ml) | 7.10 | 21.88 | 12.36 |
| At 90 minutes post-lozenge (ng/ml) | 9.11 | 27.98 | 11.23 |
| % change | +28.31% | +27.88% | −9.14% |

### Change in BES Score

FIG. 6 illustrates that subject BE1 had a decrease in score from 26 pre-administration to 24 at the 2-week follow up (7.690%); BE2 had an increase in score from 32 pre-administration to 35 at the 2-week follow up (9.380%); and BE3 had a decrease of 42 pre-administration to 28 at the 2-week follow up (33.33%); see also TABLE 7:

TABLE 7
Change in Binge Eating Scale from
Pre-Administration to 2-Week Follow Up
Change in Binge Eating Scale

| Subject | BE1 | BE2 | BE3 |
|---|---|---|---|
| Pre-administration Score | 26 | 32 | 42 |
| Score at 2-week follow up | 24 | 35 | 28 |
| % change | −7.69% | +9.38% | −33.33% |

Based on the BES, subject BE1 was classified as having moderate binge eating disorder pre-administration, and remained so at the two-week follow up; BE3 was classified as having severe binge eating disorder pre-administration, and subsequently reduced the BES by over 3300 to one point away from the moderate classification.

Subject BE3 also had the highest plasma concentrations of HNK and norketamine (244 ng/mL at 90 minutes and 975 ng/mL at 60 minutes, respectively), was the only subject to experience a decrease in total plasma ghrelin (291 pg/ml pre-administration to 274 pg/ml at 90 minutes post-lozenge, a 5.84% decrease), and had the highest reduction in BES score (42 pre-administration to 28 at the two-week follow up, a 33.33% decrease).

### Sensitivity to Reward and Sensitivity to Punishment

Sensitivity to Reward: Subject BE1 scored a total of 7; BE2 scored a total of 17; and BE3 scored a total of 2. Sensitivity to Punishment: Subject BE1 scored a total of 14; BE2 scored a total of 16; and BE3 scored a total of 16.

### Example 9: Ketamine in Subjects with Gaming Disorder

A study was completed on ketamine to treat gaming disorder. Subjects were assessed for gaming disorder using the Internet Gaming Disorder Scale (IGDS) and were included if they received an IGDS score greater than 3. Three subjects in total were included (IG1-IG3). All three were administered sublingual lozenges comprising 100 mg of racemic ketamine.

Blood samples were collected from all three subjects. Blood samples were analysed to determine ketamine and ketamine metabolite levels. Assessments were conducted as below.

### Methods

The treatment protocol commenced in the same manner as disclosed in the ketamine in subjects with gambling disorder study completed in EXAMPLE 7, with the exceptions as follow: No behavioural tasks were completed. Self-report measures completed during EXAMPLE 9 included Social Phobia Inventory (SPIN), the Craving for Gaming Questionnaire (CGQ), and Generalised Anxiety Disorder-7 (GAD-7) the Sensitivity to Reward and Punishment Scale, and the Childhood Trauma Questionnaire, the Morningness-Eveningness Questionnaire (MEQ).

Initial screening questions included: Please tell me a bit about your computer gaming. Have you spent a lot of time thinking about games or planned gaming? Do you get annoyed, uneasy, or upset when you couldn't play? Have you felt the need to play more and more? Have you tried to cut down on gaming without succeeding? Have you lost interest in previous hobbies and leisure activities because of gaming? Did you continue to play even though it created problems for you? Have you lied to family members, friends, or others about how much you have played? Did you play to reduce negative feelings (like stress, guilt, helplessness)? Have you risked or ruined an important relationship, job, education, or career opportunity because of gaming? Please tell me how old you are? Do you know how tall you are in centimetres? Do you know how much you weigh in kilograms? What is your BMI? Are you pregnant or breastfeeding (if applicable)? You would need to have a cannula fitted to the back of your hand for a large duration of the main study visit, to allow us to take blood samples at regular intervals—do you have a needle phobia? We heat your hand up first with hot water before fitting the cannula and then your hand would be kept in a hot box to keep it at a warm temperature. This all helps with getting a blood sample. Do you have any problems giving blood? You are required to fast for four hours before the main study day. We would therefore give you something to eat before you leave. Do you have any dietary requirements? Have you ever been told that you have high blood pressure? Do you have a current diagnosis of a psychiatric disorder? Does a close relative (mother, father, sister, or brother) have/had a diagnosis of a psychiatric disorder or other serious mental health condition? Have you ever had any treatment for a substance use disorder? Have you ever had a seizure that was confirmed by a medical professional? Are you taking any medication? Are you currently on any of the following medication: (barbiturates and/or narcotics, atracurium and tubocurarine, CNS depressants, thiopental, thyroid hormones, antihypertensive agents, theophylline and other methylxanthines)? Do you have any of the following: (severe coronary artery disease, storke, or brain injury; neurological condition/brain damage, intracranial mass lesions, presence of head injury or hydrocephalus, problems with memory and/or concentration, epilepsy, chronic pain, hyperthyroidism or receiving thyroid replacement, problems with chest, e.g., breathing, diabetes, liver problems, kidney problems, problems passing urine, glaucoma or globe injuries)? Do you regularly use recreational drugs? If yes, have you ever felt your use of drugs was out of control? Are you happy to proceed with the study?

### Results and Discussion

### Overall Safety and Tolerability

No unexpected adverse reactions were observed using KSET. All adverse events were transient, and resolved two hours post-lozenge. Overall, the study was well-tolerated, with no drop-outs.

### Ketamine Levels

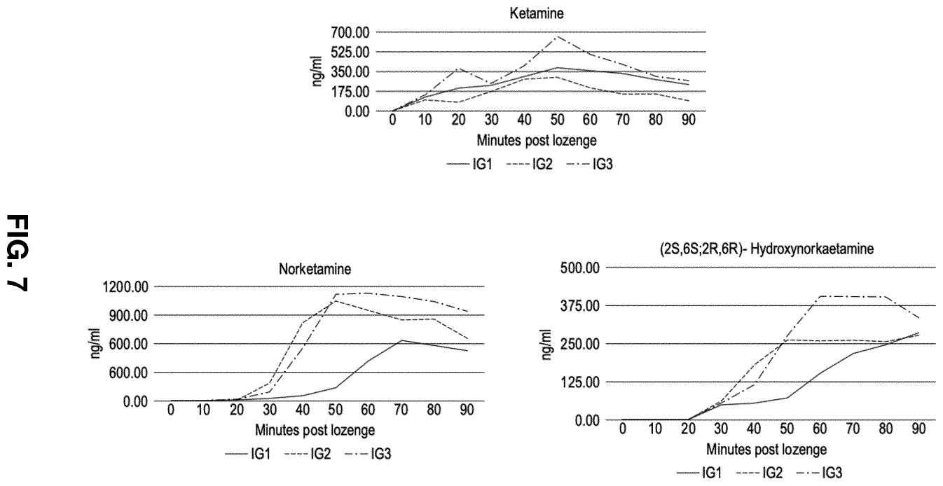

As illustrated in FIG. 7, subject IG1 had a peak concentration of about 390 ng/ml at 50 minutes post-administration; IG2 had a peak concentration of about 301 ng/ml at 50 minutes post-administration; and IG3 had a peak concentration of about 670 ng/mL at 50 minutes post-administration.

### Ketamine Metabolite Levels

(2S,6S;2R,6R)-Hydroxynorketamine (HNK) and Norketamine were each assessed.

HNK: As illustrated in FIG. 7, subject IG1 had a peak concentration of about 287 ng/ml at 90 minutes post-administration; IG2 had a peak concentration of about 279 ng/ml at 90 minutes post-administration; and IG3 had a peak concentration of about 407 ng/mL at 60 minutes post-administration.

Norketamine: As illustrated in FIG. 7, subject IG1 had a peak concentration of about 627 ng/ml at 70 minutes post-administration; IG2 had a peak concentration of about 1,040 ng/ml at 50 minutes post-administration; and IG3 had a peak concentration of about 1,116 ng/mL at 60 minutes post-administration.

### Craving for Gaming Questionnaire

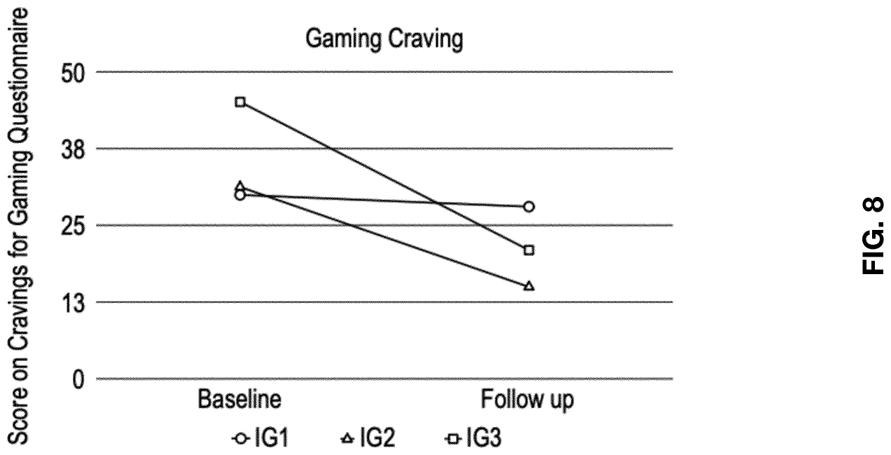

As illustrated in FIG. 8, the CGQ score for subject IG1 score decreased from 30 pre-administration to 28 at 2-week follow up (6.67%); the score for IG2 decreased from 31 pre-administration to 15 at 2-week follow up (51.61%); and the score for IG3 decreased from 45 pre-administration to 21 at 2-week follow up (53.33%); see also TABLE 8:

TABLE 8
Change in CGQ: Pre-Administration to 2-Week Follow Up
Change in Craving for Gaming Questionnaire

| Subject | IG1 | IG2 | IG3 |
|---|---|---|---|
| Pre-administration Score | 30 | 31 | 45 |
| Score at 2-week follow up | 28 | 15 | 21 |
| % change | −6.67% | −51.61% | −53.33% |

Subject IG3 had the highest reduction in CGQ score (45 pre-administration to 21 at the two-week follow up, a reduction in 53.33%), and had the highest peak plasma concentrations of HNK and norketamine (407 ng/mL at 60 minutes and 1,116 ng/mL at 60 minutes, respectively). Subject IG2 had a norketamine peak plasma concentration of 1,040 ng/ml at 50 minutes, and had a decrease in CGQ score of 31 pre-administration to 15 at the two-week follow up, a decrease of 51.61%.

### Example 10: Ketamine in Subjects with CSBD

A study was completed on the use of ketamine to treat CSBD. Subjects were assessed for CSBD using the Problematic Pornography Use Scale (PPUS) and were included if they received an PPUS score greater than 3. Three subjects in total were included (CSB1-CSB3). All three were administered sublingual lozenges comprising 100 mg of racemic ketamine.

Blood samples were collected from all three subjects. Blood samples were analysed to determine ketamine and ketamine metabolite levels. Assessments were conducted as below.

### Methods

The treatment protocol commenced in the same way as disclosed in the ketamine in subjects with gambling disorder study completed in EXAMPLE 7, with the exceptions outlined as follows: No behavioural tasks were completed. Self-report measures completed during EXAMPLE 10 included the Emotional Intimacy Measure, the Childhood Trauma Questionnaire, the Generalised Anxiety Disorder-7 (GAD-7), the Morningness-Eveningness Questionnaire (MEQ), and the Problematic Pornography Questionnaire (PPQ), the Acceptance and Action Questionnaire, and the Psychological and Interpersonal Relationship Scales (PAIRS).

### Results and Discussion

### Overall Safety and Tolerability

No unexpected adverse reactions were observed using the KSET. All adverse events were transient, and resolved two hours post-lozenge. Overall, the study was well-tolerated, with no drop-outs.

### Ketamine Levels

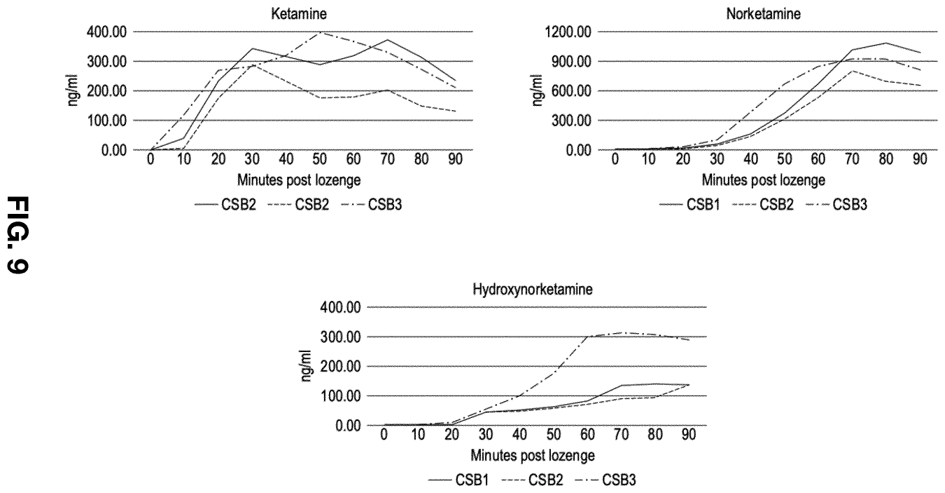

As illustrated in FIG. 9, subject CSB1 had a peak concentration of about 368 ng/ml at 70 minutes post-administration; CSB2 had a peak concentration of about 285 ng/ml at 30 minutes post-administration, and subject CSB3 had a peak concentration of about 395 ng/mL at 50 minutes post-administration.

### Ketamine Metabolite Levels

(2S,6S;2R,6R)-Hydroxynorketamine (HNK) and Norketamine were each assessed.

HNK: As illustrated in FIG. 9, subject CSB1 had a peak concentration of about 136 ng/ml at 80 minutes post-administration, subject CSB2 had a peak concentration of about 135 ng/ml at 90 minutes post-administration, and subject CSB3 had a peak concentration of about 314 ng/mL at 70 minutes post-administration.

Norketamine: As illustrated in FIG. 9, subject CSB1 had a peak concentration of about 1,069 ng/ml at 80 minutes post-administration, subject CSB2 had a peak concentration of about 785 ng/ml at 70 minutes post-administration, and subject CSB3 had a peak concentration of about 923 ng/mL at 70 minutes post-administration.

### Problematic Pornography Use Scale

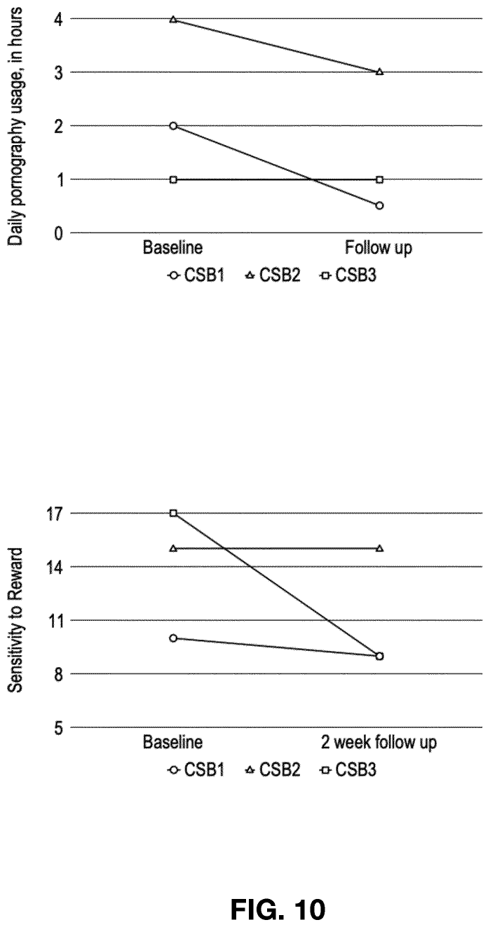

As illustrated in FIG. 10, hours of pornography viewed per day by subject CSB1 decreased from 2 hours pre-administration to 0.5 hours at the 2-week follow up (75%); hours of pornography viewed per day by CSBG2 decreased from 4 hours pre-administration to 3 hours at the 2-week follow up (25%); and hours of pornography viewed per day by CSBG3 remained constant at 1 hour; see also TABLE 9:

TABLE 9
Hours of Pornography Viewed: Pre-Administration to 2-Week Follow Up
Change in Hours of Pornography Viewed

| Subject | CSB1 | CSB2 | CSB3 |
|---|---|---|---|
| Hours watched daily Pre-administration | 2 | 4 | 1 |
| Hours watched daily at 2-week follow up | 0.5 | 3 | 1 |
| % change | −75% | −25% | N/A |

### Sensitivity to Reward

As illustrated in FIG. 10, subject CSB1 had a decrease from 10 pre-administration to 9 at the 2-week follow up (10%); CSB2 remained the same, at 15 pre-administration and 15 at the 2-week follow up; and CSB3 had a decrease from 17 pre-administration to 9 at the 2-week follow up (47.06%); see also TABLE 10:

TABLE 10
Change in Sensitivity to Reward:
Pre-Administration to 2-Week Follow Up
Change in Sensitivity to Reward

| Subject | CSB1 | CSB2 | CSB3 |
|---|---|---|---|
| Sensitivity to Reward Pre-administration | 10 | 15 | 17 |
| Sensitivity to Reward at 2-week follow up | 9 | 15 | 9 |
| % change | −10% | N/A | −47.06% |

Subject CSB1 had the largest reduction in hours of pornography viewed per day (2 hours/day pre-administration to 0.5 hours/day at the two-week follow up, a 75% decrease), and had the highest peak plasma concentration of norketamine (1,069 ng/ml at 80 minutes). This reduction persisted for 2 weeks post-administration.

Subject CSB3 had the largest decrease in sensitivity to reward score (17 pre-administration to 9 after the two-week follow up, a 47.06% reduction), and had the highest peak plasma concentration of HNK (314 ng/mL at 70 minutes).

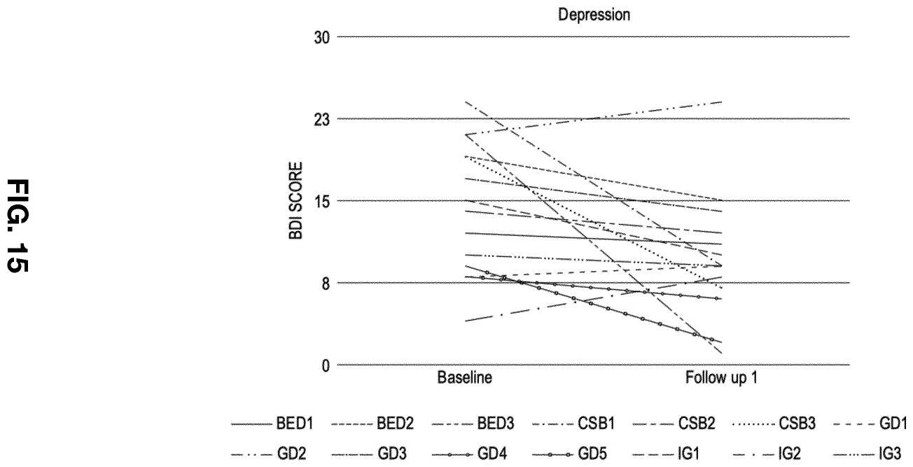

FIG. 15 illustrates the increase in depression scores using the Beck Depression Index, from baseline to week follow-up, calculated for each subject of each study in EXAMPLES 7-10. Subject G1 had a baseline score of 8 and a score of 9 at the one-week follow up (12.5% increase), subject G2 had a baseline score of 21 and a score of 24 at the one-week follow up (14.29% increase), subject G3 had a baseline score of 17 and a score of 14 at the one-week follow up (17.65% decrease), subject G4 had a baseline score of 8 and a score of 6 at the one-week follow up (25 decrease), subject G5 had a baseline score of 12 and a score of 11 at the one-week follow up (8.3300 decrease), subject BE1 had a baseline score of 9 and a score of 2 at the one-week follow up (77.78% decrease), subject BE2 had a baseline score of 19 and a score of 15 at the one-week follow up (21.0% decrease), subject BE3 had a baseline score of 21 and a score of 1 at the one-week follow up (95.24% decrease), subject IG1 had a baseline score of 15 and a score of 10 at the one-week follow up (33.33% decrease), subject IG2 had a baseline score of 4 and a score of 8 at the one-week follow up (10000 increase), subject IG3 had a baseline score of 10 and a score of 9 at the one-week follow up (100% decrease), subject CSB1 had a baseline score of 24 and a score of 9 at the one-week follow up (62.50% decrease), subject CSB2 had a baseline score of 14 and a score of 12 at the one-week follow up (14.292 decrease), and subject CSB3 had a baseline score of 19 and a score of 7 at the one-week follow up (63.16% decrease); see also TABLE 11:

TABLE 11
Change in BDI Score from Pre-Treatment, to One-Week Follow Up
Change in Beck Depression Index (BDI) Score

| | BDI Score | BDI Score at the | |
|---|---|---|---|
| Subject | pre-treatment | one-week follow up | % Change |
| G1 | 8 | 9 | +12.5% |
| G2 | 21 | 24 | +14.29% |
| G3 | 17 | 14 | −17.65% |
| G4 | 8 | 6 | −25% |
| G5 | 12 | 11 | −8.33% |
| BE1 | 9 | 2 | −77.78% |
| BE2 | 19 | 15 | −21.05% |
| BE3 | 21 | 1 | −95.24% |
| IG1 | 15 | 10 | −33.33% |
| IG2 | 4 | 8 | +100% |
| IG3 | 10 | 9 | −10% |
| CSB1 | 24 | 9 | −62.50% |
| CSB2 | 14 | 12 | −14.29% |
| CSB3 | 19 | 7 | −63.16% |

Five subjects decreased from a greater to lesser level of severity according to BDI results. Subject BE3 decreased from moderate to minimal; IG1 decreased from mild to minimal; CSB1 decreased from moderate to minimal; CSB2 decreased from mild to minimal; and CSB3 decreased from mild to minimal; see also TABLE 12:

TABLE 12
Decreases From Greater to a Lesser Level of BDI Severity
Decreases in Beck Depression Index Severity Levels

| | Depression Severity | Depression Severity at the One |
|---|---|---|
| Subject | Pre-Treatment | Week Follow Up |
| BE3 | Moderate | Minimal |
| IG1 | Mild | Minimal |
| CSB1 | Moderate | Minimal |
| CSB2 | Mild | Minimal |
| CSB3 | Mild | Minimal |

### CSB3 Mild Minimal

Subjects G1, G2, and IG2 showed improvements in treatment for gambling disorder, and gaming disorder, respectively, but had higher BDI scores post-treatment.

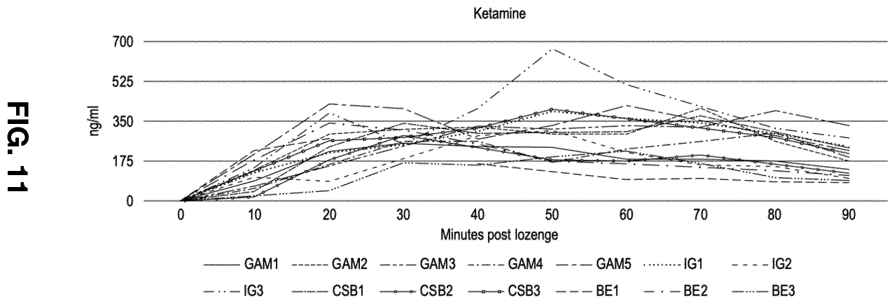

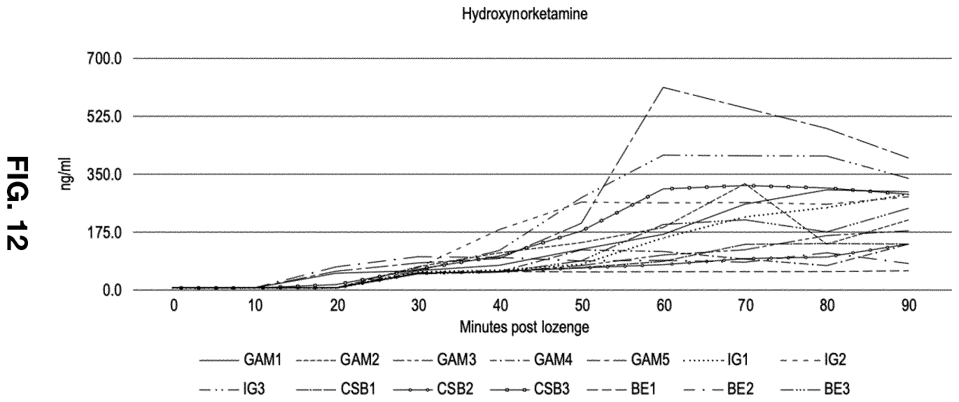

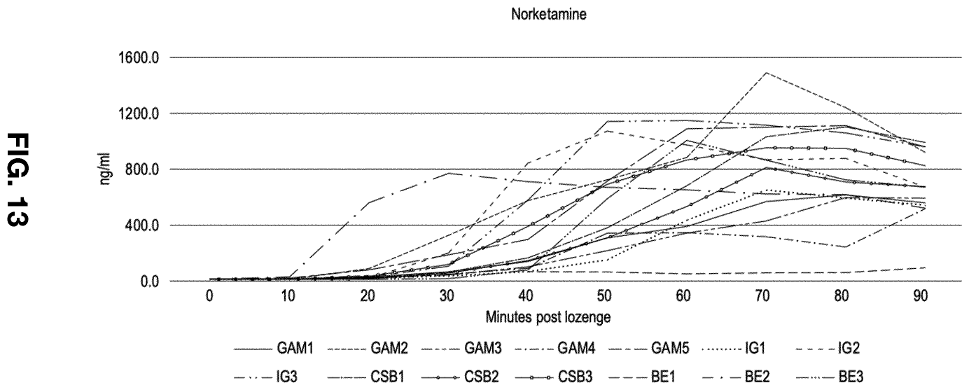

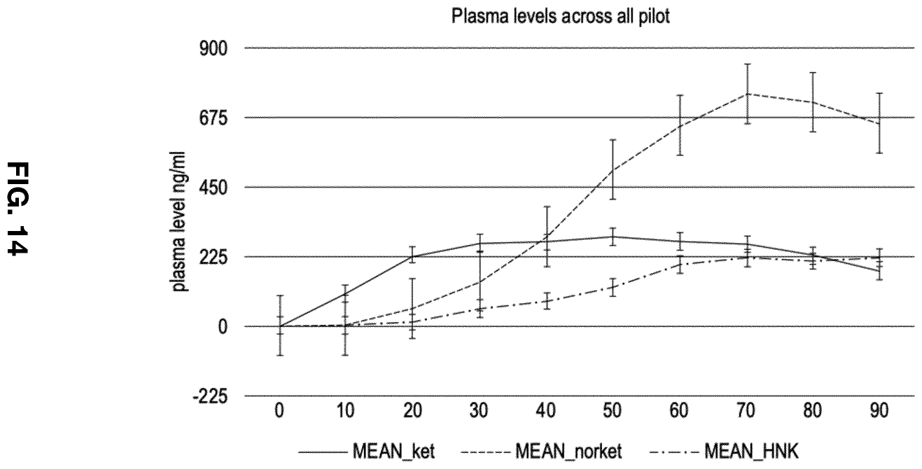

FIGS. 11-13 illustrate ketamine, HNK, and norketamine levels, respectively, for all subjects of EXAMPLES 7-10, while FIG. 14 illustrates the mean plasma levels of ketamine, HNK, and norketamine for all subjects of EXAMPLES 7-10. Results indicated a mean plasma concentration of ketamine of 104.87 ng/ml at 10 minutes, 230.11 ng/ml at 20 minutes, 267.08 ng/ml at 30 minutes, 271.62 ng/ml at 40 minutes, 288.48 ng/ml at 50 minutes, 272.42 ng/ml at 60 minutes, 263.40 ng/ml at 70 minutes, 227.50 ng/ml at 80 minutes, and 176.91 ng/ml at 90 minutes. Mean HNK was 0 at 10 minutes, 12.22 ng/ml at 20 minutes, 56.73 ng/ml at 30 minutes, 79.10 ng/ml at 40 minutes, 126.20 ng/ml at 50 minutes, 198.87 ng/ml at 60 minutes, 221.08 ng/ml at 70 minutes, 209.14 ng/ml at 80 minutes, and 217.32 ng/ml at 90 minutes. Mean norketamine was 1.92 at 10 minutes, 57.39 ng/ml at 20 minutes, 141.78 ng/ml at 30 minutes, 286.02 ng/ml at 40 minutes, 505.15 ng/ml at 50 minutes, 646.73 ng/ml at 60 minutes, 750.57 ng/ml at 70 minutes, 723.79 ng/ml at 80 minutes, and 653.94 ng/ml at 90 minutes. This information is also illustrated in TABLE 13:

TABLE 13
Mean Ketamine, HNK, and Norketamine Post-Administration
Mean Ketamine, HNK, and Norketamine Post-Administration

| | Compound | | |
|---|---|---|---|
| Time Post- | Ketamine | HNK | Norketamine |
| administration | (ng/ml) | (ng/ml) | (ng/ml) |
| 10 Minutes | 104.87 | 0 | 1.92 |
| 20 Minutes | 230.11 | 12.22 | 57.39 |
| 30 Minutes | 267.08 | 56.73 | 141.78 |
| 40 Minutes | 271.62 | 79.10 | 286.02 |
| 50 Minutes | 288.48 | 126.20 | 505.15 |
| 60 Minutes | 272.42 | 198.87 | 646.73 |
| 70 Minutes | 263.40 | 221.08 | 750.57 |
| 80 Minutes | 227.50 | 209.14 | 723.79 |
| 90 Minutes | 176.91 | 217.32 | 653.94 |

### E. Prophetic Examples

That the disclosed methods are useful in treating behavioural addictions is shown in part by the following Examples. While every attempt is made to use the present and future tenses, for avoidance of doubt, these Examples, and any examples discussed elsewhere are prophetic examples (even if not so labeled, and unless stated otherwise, such as Examples 7-10 where a ketamine composition is described as having been administered). It should be understood that these Examples are merely exemplary and illustrative and not limiting.

### Example 11: Long-Term Behavioural Addiction Study

An exemplary long-term behavioural addiction study may proceed in the same manner as the studies of EXAMPLES 7-10. For instance, an exemplary long-term gambling disorder study may proceed in the same manner as EXAMPLE 7. An exemplary long-term binge eating disorder study may proceed in the same manner as EXAMPLE 8. An exemplary long-term gaming disorder study may proceed in the same manner as EXAMPLE 9. An exemplary long-term CSBD study may proceed in the same manner as EXAMPLE 10.

An exemplary long-term behavioural addiction study also may proceed in a similar manner as the studies of EXAMPLES 7-10, and include subjects with at least one behavioural addiction, including one or more of gambling disorder, gaming disorder, compulsive sexual behaviour disorder, binge eating disorder, compulsive buying-shopping disorder, technology addiction, kleptomania, pyromania, internet addiction, pornography addiction, and such other disorders disclosed herein and known to those of skill.

Besides being assessed as discussed in EXAMPLES 7-10, subjects also will be assessed again at any one or more of 1 month, 2 months, 3 months, 4 months, 5 months, 6 months, 7 months, 8 months, 9 months, 10 months, 11 months, 1 year, 1.5 years, and 2 years post-treatment. The one or more post-treatment assessments will be compared to baseline.

In an exemplary long-term gambling disorder study, decreases in PGSI score will remain below about 80% of baseline, including 75%, 70%, 65%, 60%, 55%, 50%, 45%, 40%, 35%, 30%, 25%, 20%, 15%, 10%, 5%, and less than 5% below baseline, when measured at post-treatment, wherein “at post-treatment” refers to any one or more of the post-treatment assessments. In an exemplary long-term binge eating disorder study, decreases in BES score will remain below about 80% of baseline, including 75%, 70%, 65%, 60%, 55%, 50%, 45%, 40^{%}, 35%, 30%, 25%, 20%, 15%, 10%, 5%, and less than 5% below baseline, when measured at post-treatment. In an exemplary long-term gaming disorder study, decreases in CGQ score will remain below about 80% of baseline, including 75%, 70%, 65%, 60%, 55%, 50%, 45%, 40%, 35%, 30%, 25%, 20%, 15%, 10%, 5%, and less than 5% below baseline, when measured at post-treatment. In an exemplary long-term CSBD study, decreases in hours of pornography viewed and/or change in sensitivity to reward will remain below about 80% of baseline, including 75%, 70%, 65%, 60%, 55%, 50%, 45%, 40%, 35%, 30%, 25%, 20%, 15%, 10%, 5%, and less than 5% below baseline, when measured at post-treatment.

A behavioural addiction may be characterised as being any of mild, moderate, and severe. The severity of the disorder may be determined by means known to those of skill, such as the assessments disclosed herein. A subject in an exemplary study also may have one or more comorbid psychiatric conditions.

The subject in the study of this Example is administered racemic ketamine. In embodiments, the subject is administered a composition comprising ketamine, a pure or substantially pure ketamine enantiomer, an enantiomerically enriched mixture of ketamine, a ketamine metabolite, or a ketamine analog. In embodiments, the subject will receive KAP.

This Example will demonstrate that the administration of ketamine, the administration of a composition comprising ketamine, a pure or substantially pure ketamine enantiomer, an enantiomerically enriched mixture of ketamine, a ketamine metabolite, or a ketamine analog, including where any of the foregoing is provided with KAP, is effective to treat the behavioural addiction. That such administration is effective to treat the behavioural addiction may be shown by a reduction in the severity of the behavioural addiction.

In embodiments, a subject who has a behavioural addiction characterised as being mild when measured at baseline will no longer have a mild behavioural addiction. In embodiments, a subject who has a behavioural addiction characterised as being moderate when measured at baseline will no longer have a moderate behavioural addiction. In embodiments, a subject who has a behavioural addiction characterised as being severe when measured at baseline will no longer have a severe behavioural addiction.

In embodiments, this Example will demonstrate that the severity of the subject's diagnosis is reduced. That the severity of the subject's diagnosis is reduced will be shown in subjects with at least one behavioural addiction, including one ore more of gambling disorder, gaming disorder, compulsive sexual behaviour disorder, binge eating disorder, compulsive buying-shopping disorder, technology addiction, kleptomania, pyromania, internet addiction, pornography addiction, and such other disorders disclosed herein and known to those of skill.

In embodiments, the severity of the subject's diagnosis may be assessed at any one or more of 1 month, 2 months, 3 months, 4 months, 5 months, 6 months, 7 months, 8 months, 9 months, 10 months, 11 months, 1 year, 1.5 years, and 2 years post-treatment.

In embodiments, a subject classified as a problem gambler pre-administration and any of a low-risk gambler and moderate-risk gambler at the initial two-week follow up will remain either of a low-risk gambler or a moderate-risk gambler when assessed again at any of 1 month, 2 months, 3 months, 4 months, 5 months, 6 months, 7 months, 8 months, 9 months, 10 months, 11 months, 1 year, 1.5 years, and 2 years post-treatment. Likewise, in embodiments, a subject classified as a moderate-risk gambler pre-administration and a low-risk gambler at the initial two-week follow up will remain a low-risk gambler when assessed again at any of 1 month, 2 months, 3 months, 4 months, 5 months, 6 months, 7 months, 8 months, 9 months, 10 months, 11 months, 1 year, 1.5 years, and 2 years post-treatment. In embodiments, a subject classified as a low-risk gambler pre-administration who no longer qualified for the diagnosis at the initial two-week follow up will remain without a diagnosis when assessed again at any of 1 month, 2 months, 3 months, 4 months, 5 months, 6 months, 7 months, 8 months, 9 months, 10 months, 11 months, 1 year, 1.5 years, and 2 years post-treatment.

In embodiments, a subject classified as having severe binge eating disorder pre-administration and any of mild binge eating disorder and moderate binge eating disorder at the initial two-week follow up will continue to have either of mild binge eating disorder or moderate binge eating disorder when assessed again at any of 1 month, 2 months, 3 months, 4 months, 5 months, 6 months, 7 months, 8 months, 9 months, 10 months, 11 months, 1 year, 1.5 years, and 2 years post-treatment. In embodiments, a subject classified as having moderate binge eating disorder pre-administration and mild binge eating disorder at the initial two-week follow up will continue to have mild binge eating disorder when assessed again at any of 1 month, 2 months, 3 months, 4 months, 5 months, 6 months, 7 months, 8 months, 9 months, 10 months, 11 months, 1 year, 1.5 years, and 2 years post-treatment. In embodiments, a subject classified as having mild binge eating disorder pre-administration and who no longer qualified for the diagnosis at the initial two-week follow up will continue to be without the diagnosis when assessed again at any of 1 month, 2 months, 3 months, 4 months, 5 months, 6 months, 7 months, 8 months, 9 months, 10 months, 11 months, 1 year, 1.5 years, and 2 years post-treatment.

In embodiments, a subject classified as having severe gaming disorder pre-administration and any of mild gaming disorder and moderate gaming disorder at the initial two-week follow up will continue to have either of mild gaming disorder or moderate gaming disorder when assessed again at any of 1 month, 2 months, 3 months, 4 months, 5 months, 6 months, 7 months, 8 months, 9 months, 10 months, 11 months, 1 year, 1.5 years, and 2 years post-treatment. In embodiments, a subject classified as having moderate gaming disorder pre-administration and mild gaming disorder at the initial two-week follow up will continue to have mild gaming disorder when assessed again at any of 1 month, 2 months, 3 months, 4 months, 5 months, 6 months, 7 months, 8 months, 9 months, 10 months, 11 months, 1 year, 1.5 years, and 2 years post-treatment. In embodiments, a subject classified as having mild gaming disorder pre-administration and who no longer qualified for the diagnosis at the initial two-week follow up will continue to be without the diagnosis when assessed again at any of 1 month, 2 months, 3 months, 4 months, 5 months, 6 months, 7 months, 8 months, 9 months, 10 months, 11 months, 1 year, 1.5 years, and 2 years post-treatment.

In embodiments, when a subject is assessed at any of 1 month, 2 months, 3 months, 4 months, 5 months, 6 months, 7 months, 8 months, 9 months, 10 months, 11 months, 1 year, 1.5 years, and 2 years post-treatment, the administration of ketamine according to the disclosed methods will be effective to treat the behavioural addiction as shown by one or more of (a) a reduction in the behaviour, (b) a reduction in drive or urge to engage in the behaviour, (c) a promotion of abstinence from the behaviour, (d) a prevention of relapse into the behaviour, (e) a reduction in at least one symptom of the behavioural addiction.

In embodiments, administration of ketamine according to the disclosed methods will result in a measurable reduction in a behavioural addiction or in the drive or urge to engage in the behaviour, for example as compared to a baseline determination made before such administration. In embodiments, the reduction in the behaviour and/or the reduction in drive or urge to engage in the behaviour is at least 15%, including 20%, 25%, 30%, 40%, 50%, 60%, 70%, 80%, 90%, and up to and including 100%, when measured from baseline.

In embodiments, administration of ketamine according to the disclosed methods will result in a durable promotion of abstinence from the behaviour or prevention of relapse into the behaviour, when measured from the start of treatment, such as the first administration. In embodiments, the promotion of abstinence from the behaviour results in abstinence for at least 1 month, including 2 months, 3 months, 4 months, 5 months, 6 months, 7 months, 8 months, 9 months, 10 months, 11 months, one year, or more than 1 year, including 1.5 years, 2 years, and more than 2 years from the start of treatment. In embodiments, the reduction in the behaviour, reduction in drive or urge to engage in the behaviour, promotion of abstinence from the behaviour, prevention of relapse into the behaviour, and reduction in at least one symptom of the behavioural addiction lasts for at least about 1 month, including 2 months, 3 months, 4 months, 5 months, 6 months, 7 months, 8 months, 9 months, 10 months, 11 months, one year, or more than 1 year, including 1.5 years, 2 years, and more than 2 years.

In embodiments, administration of ketamine according to the disclosed methods will result in a measurable improvement in at least one symptom of a behavioural addiction, for example as compared to a baseline determination made before such administration. In embodiments, the reduction in at least one symptom of the behavioural addiction is a reduction in at least about 15%, including 20%, 25%, 30%, 40%, 50%, 60%, 70%, 80%, 90%, and up to and including 100%, when measured from baseline.

In embodiments, administration of ketamine according to the disclosed methods will result in a reduction in at least one symptom of a comorbid psychiatric condition. In embodiments, the reduction in at least one symptom of the comorbid psychiatric condition is a reduction in at least about 15%, including 20%, 25%, 30%, 40%, 50%, 60%, 70%, 80%, 90%, and up to and including 100%, when measured from baseline.

### Example 12: Ketamine in Subjects with Gambling Disorder

Purpose: Aims of this mechanistic study include to examine the impact of a single dose of intranasal (IN) ketamine, which may be racemic ketamine, esketamine, or another disclosed ketamine composition, in people with gambling use disorder on reward sensitivity and processing and on habitual responding, and to examine activation reward sensitivity and processing two hours following ketamine administration on a neuroimaging version of the reward processing task. Markers of effectiveness will be changes in reward processing, and attenuation of response on no-win trials, in particular response to “near-misses.” Other markers of craving, psychological wellbeing, and gambling severity are secondary outcomes. Preliminary explorations of the sub-acute impact of ketamine on our procedural learning task would seek to examine whether ketamine can disrupt striatal habitual responding.

Subjects and Design: Forty subjects meeting DSM-5 criteria for gambling use disorder will be identified and randomised to receive either IN ketamine or IN placebo midazolam. Inclusion criteria will be over 18 years old; no serious mental health problems; and no contraindications for ketamine (hypertension, ketamine use disorder or diagnosis of a substance use disorder, taking prescribed contraindicated medications).

Procedure: Following telephone screening by the research team, in person screening by a medical physician and psychologist, and informed consent, demographic data will be collected from subjects. Subjects will then undergo baseline measures of reward (slot machine task and monetary incentive delay) and procedural learning (serial reaction time task), alongside questionnaires of mood, gambling severity, trauma history and psychological wellbeing. Ketamine dosing will be via procedures used in administration of IN esketamine.

Subjects will recline listening to music for the duration of the acute effect and then complete questionnaires of mood, wellbeing, dissociative symptoms, and mystical effects. The same cognitive tasks will be repeated and subjects will be moved to the MRI/PET unit to undergo an fMRI scan while completing a virtual slot machine task in the scanner.

Results: This study will show that a single dose of intranasal (IN) ketamine is an effective treatment for subjects with a behavioural addiction. The study of this Example will show that ketamine is superior to placebo, as measured by any one or more of: (1) frequency of the compulsive or problematic behaviour, (2) drive or urge to engage in the compulsive or problematic behaviour, (3) abstinence from the compulsive or problematic behaviour, (4) relapse into the compulsive or problematic behaviour, (5) severity of symptoms of the compulsive or problematic behaviour, (6) time spent on the compulsive or problematic behaviour; (7) quality of life, including subjective sleep; and (8) psychosocial functioning.

### Example 13: Exemplary KAP Treatment Regimen

In certain exemplary treatment methods, the disclosed ketamine compositions are administered together as part of a KAP treatment regimen, wherein “treatment regimen” refers to a series or course of KAP. One exemplary KAP treatment regimen takes place over six weeks. Another exemplary KAP treatment regimen takes place over four weeks. Additional exemplary KAP treatment regimens will be understood, e.g., a patient may undergo only a single session, a course having a single drug-assisted session and integration session, a course having three drug-assisted sessions and between two and four (or more) additional psychotherapeutic non-drug sessions, and other variations as readily appreciated.

In one embodiment of an exemplary KAP treatment regimen, the ketamine dose will be titrated according to individual response. This will allow the therapeutic relationship to be well established before the patient considers opting for higher doses which facilitates a transformational state with increasing opportunity for dissociative experiences.

After an initial course, patients may have a final follow-up session, 1-3 weeks later. If clinical judgment suggests value, they may be offered a repeat of the full course again, the option of a shorter course, or further single “booster” (i.e., any additional course or session following a first such course or session) sessions of ketamine, with accompanying single sessions of preparation and post-drug psychotherapy at less frequent intervals (e.g., monthly).

In some embodiments, a patient may be self-referred or may receive a referral from a health professional. In embodiments, there may be a “pre-assessment” triage and review of the referral (whether a self-referral or referral by a health professional) by the clinical team. In embodiments, patients with a behavioural addiction that includes a gambling disorder wishing to undergo KAP may be screened prior to psychotherapy. Eligible patients may, in embodiments, attend a baseline visit after screening to ensure eligibility. In embodiments, exemplary inclusion and exclusion criteria may be those outlined in TABLE 15 below.

TABLE 15
Exemplary Inclusion and Exclusion Criteria
Exemplary Inclusion Criteria

| | |
|---|---|
| 1 | Informed consent |
| 2 | Primary diagnosis (as defined by DSM-5) of gambling disorder (or diagnosis of another |
| | behavioural addiction) |
| 3 | Between 18 and 65 years old |
| 4 | Proficient in speaking and reading the language in which the therapy is provided |
| 5 | Agree to comply with requirements of therapy protocol |
| 6 | Be able to identify in advance a supportive friend, family member, or significant other: |
| | who could meet with the clinician and receive information about the treatment at |
| | one of the preparation sessions prior to the patient's first ketamine session |
| | who could accompany the patient to study visits if required |
| | who could act as a “locator” and be contacted by the therapy team in order to remind the |
| | patient about follow-up appointments or collect outcome data (such as drinking |
| | behaviour) in the event that the patient themselves cannot be contacted |

Exemplary Exclusion Criteria

| | |
|---|---|
| 1 | Lacking capacity (people who cannot give informed consent, e.g., have a serious mental |
| | impairment) |
| 2 | Patients who refuse to give a medical history |
| 3 | People aged below 18 or over 65 years old |
| 4 | Patients who refuse to give details of other health care professionals (e.g., GPS, |
| | consultant, etc.) |
| 5 | Known violent or aggressive people |
| 6 | People who are not regarded as able to benefit from ketamine therapy |
| 7 | Any of the following psychiatric conditions: |
| | Schizophrenia |
| | Schizoaffective disorder |
| | Other Psychotic disorders |
| | Severe Personality disorders |
| | Active manic episode |
| | Ketamine Use Disorder |
| 8 | Any of the following medical conditions: |
| | Uncontrolled Hypertension |
| | Untreated Hyperthyroidism |
| | Congestive Heart Failure (decreased clearance) |
| | Severe Coronary Disease (Angina) |
| | Dysrhythmias (WPW, frequent SVT, uncontrolled AF) |
| | Severe Liver Disease |
| | Severe Kidney Disease |
| | Elevated ICP (mass) |
| | Elevated IOP- Uncontrolled Glaucoma |
| | Pregnancy/Breastfeeding |
| | Airway concerns: severe OSA, mass |
| 9 | People with serious comorbidities e.g., advanced carcinoma |
| 10 | Present a serious suicide risk, e.g., as determined using the clinical judgment of one or |
| | more qualified mental health professionals within a therapy team. Risk may be |
| | determined using information from the Columbia-Suicide Severity Risk Scale (C-SSRS) |
| | which allows classification of severity of suicidal ideation and behaviour. The C-SSRS |
| | scale classifies severe risk as (a) current suicidal ideation with intent and/or plan, and (b) |
| | suicidal behaviour in the last 3 months. A clinical judgment regarding the level of risk |
| | and subsequent decisions regarding eligibility and care could use a combination of the |
| | information provided by the C-SSRS, the patient's history of previous risk behaviours, |
| | any presenting mental health difficulties, and environmental and clinical factors. A final |
| | decision may include a discussion with the qualified mental health professionals within |
| | the therapy team. |
| 11 | People who fall outside the indications for therapy in a selected clinical protocol |

Exclusion criteria also may be determined by the therapy team through the use of their clinical judgment. In some embodiments, a patient will not be administered a ketamine composition or undergo in a KAP treatment regime based on one or more exclusion criteria.

In embodiments, potential patients may be invited to a screening visit to determine their eligibility for KAP using the disclosed methods. In embodiments, the screening visit may include, e.g., informed consent, medical, psychiatric and substance misuse history, prescribed medication use, basic physiological observations, urine drugs test and optional alcohol test, and other screens as clinically indicated. In order to monitor the risk of patients using ketamine outside of the therapeutic context, they can be asked about their use of, or desire to use, illicit ketamine (e.g., with standard questions such as used by MAPS when asking about illicit ecstasy use in MDMA-assisted therapy studies).

In embodiments, the therapeutic process may be discussed and explained during screening. In embodiments, the screening may be in person, may be conducted via telemedicine, e.g., via telephone, a video call, a text chat, etc. Potential patients also may be contacted and partake in a short telephone screen before invitation to a full screening visit.

In embodiments, if a psychiatric assessment is sought, it may be performed by a trained psychiatrist. Assessments may include, as non-limiting examples, those disclosed in TABLE 2, to screen for comorbid psychiatric conditions and clinical risk assessments for suicidality. Clinical data collection may include years spent performing a particular compulsive or problematic behaviour, complication and consequences arising from that behaviour, and previous therapeutic inputs that have been provided. Other questionnaires also may be completed. In embodiments, pre-study baseline questionnaires may also be completed. Information about practices and patterns of the compulsive or problematic behaviour previous to the screening visit may be assessed, e.g., for the three months prior to screening for Gambling Disorder patients using the Gambling Timeline Follow Back (G-TLFB) method (Sobell 2001), a validated assessment using a calendar method and memory aids to collect retrospective estimates about gambling frequency, and to provide subjective ratings of each episode's intensity (i.e., heavy, moderate, or light). Patients may be given a calendar to take away to help track their behaviour for the duration of the therapy.

In embodiments, patients may attend a baseline visit to confirm eligibility for continuation into KAP. In embodiments, this may include a medical assessment session with the patient to, e.g., gather further clinical details, assess eligibility, and discuss appropriate bespoke dosing regimens dependent upon the individual patient's specific needs and past experience. If necessary or desired, a brief physical examination and ECG may be performed, and blood samples for routine laboratory tests (urea and electrolytes, full blood count, liver function tests and Gamma-GT) also may be completed. Following this initial medical assessment, a patient may begin a KAP treatment regimen. In other embodiments, a patient may begin a KAP treatment regimen directly, such as after being referred by a physician.

### KAP Treatment Regimen—Four Ketamine Administration Sessions

In an exemplary KAP treatment regime, a patient may complete between about one to about 20 or more total therapy sessions during a treatment range lasting between about one and about 15 weeks. In embodiments, the KAP treatment regime includes one 60-minute preparatory psychotherapy session, two half-day KAP sessions (wherein, in embodiments, each is followed by a 60-minute non-drug integration psychotherapy session), one 60-minute mid-way preparation and integration psychotherapy session, and two additional half-day KAP sessions (which, in embodiments, are each followed by a 60-minute non-drug integration psychotherapy session). Optionally, the protocol may include an additional 60-minute follow-up sessions).

This exemplary 11 session KAP treatment regime may be understood further by reference to TABLE 16 below. An additional exemplary four-week course follows (which may be used as a supplemental course, following a longer course, or a standalone shorter course of KAP), and additional courses will be within the practice of ordinary skill, to an ordinary artisan having knowledge of the teachings herein, such as an eight week or longer course (having five or more KAP sessions), a course having three KAP sessions, or a minimum course having only a single KAP session, and numerous other like variations.

Likewise, while 60 minutes is the length of time given for each non-dosing session, such a timeframe is merely exemplary, and is not a limitation. In embodiments, the non-dosing sessions may be any length of time necessary to complete the tasks associated with that session, including between about 1 minute and about 300 minutes, inclusive. Moreover, while dosing sessions state the length of time will be “half a day” such times may vary depending on a variety of factors, including patient response to ketamine, the length of time preparation and administration take, the ketamine dose administered, the patient's metabolism (e.g., how long it takes the patient to eliminate ketamine), and such other factors that will be apparent to one of skill. In embodiments, KAP (i.e., ketamine administration) sessions may last from between about 2 hours to about 12 hours, inclusive.

TABLE 16
Exemplary Six-Week KAP Treatment Regimen

| Week | Session | Activity | Length | Comments: |
|---|---|---|---|---|
| 1 | 1 | Prep Session | 60-minutes, | Non-drug assisted preparation psychotherapy is |
| | | | then home | used to further discuss the patient's |
| | | | | psychological issues and establish their |
| | | | | intention for the therapeutic course. |
| 2 | 2 | K1 IM dose | Half-Day, | Suicide clinical risk assessment is carried out. |
| | | then home | then home | Brief psychotherapy is given before, during and |
| | | | | after the dose. The patient remains in the clinic |
| | | | | in the recovery area until deemed medically fit |
| | | | | for discharge. They will be instructed not to |
| | | | | drive home, but rather be accompanied by a |
| | | | | designated person. |
| 2 | 3 | Integration | 60-minutes, | Non-drug integration to discuss the material |
| | | Day after K1 | then home | that emerged during yesterday's session. |
| | | | | Decision is made in collaboration with the |
| | | | | patient about progressing to a higher dose for |
| | | | | the next session. |
| 3 | 4 | K2 IM dose | Half-Day, | Suicide clinical risk assessment is carried out. |
| | | then home | then home | Brief psychotherapy is given before, during and |
| | | | | after the dose. The patient remains in the clinic |
| | | | | in the recovery area until deemed medically fit |
| | | | | for discharge. They will be instructed not to |
| | | | | drive home, but rather be accompanied by a |
| | | | | designated person. |
| 3 | 5 | Integration | 60-minutes, | Non-drug integration to discuss the material |
| | | Day after Kl | then home | that emerged during yesterday's session. |
| | | | | Decision is made in collaboration with the |
| | | | | patient about progressing to a higher dose for |
| | | | | the next session. |
| 4 | 6 | Prep/ | 60-minutes, | Non-drug assisted preparation psychotherapy is |
| | | Integration | then home | used to further discuss the patient's |
| | | Session | | psychological issues and establish their |
| | | | | intention for the therapeutic course. |
| 5 | 7 | K3 IM dose | Half-Day, | Suicide clinical risk assessment is carried out. |
| | | then home | then home | Brief psychotherapy is given before, during and |
| | | | | after the dose. The patient remains in the clinic |
| | | | | in the recovery area until deemed medically fit |
| | | | | for discharge. They will be instructed not to |
| | | | | drive home, but rather be accompanied by a |
| | | | | designated person. |
| 5 | 8 | Next day | 60-minutes, | Non-drug integration to discuss the material |
| | | integration | then home | that emerged during yesterday's session. A |
| | | | | decision is made in collaboration with the |
| | | | | patient about progressing to a higher dose for |
| | | | | the next session. |
| 6 | 9 | K4 IM dose | Half-Day, | Suicide clinical risk assessment is carried out. |
| | | then home | then home | Brief psychotherapy is given before, during and |
| | | | | after the dose. The patient remains in the clinic |
| | | | | in the recovery area until deemed medically fit |
| | | | | for discharge. They will be instructed not to |
| | | | | drive home, but rather be accompanied by a |
| | | | | designated person. |
| 6 | 10 | Next day | 60-minutes, | Non-drug integration to discuss the material |
| | | Final | then home | that emerged during yesterday's session. A |
| | | integration | | decision is made in collaboration with the |
| | | | | patient about progressing to a higher dose for |
| | | | | the next session. |
| (9) | (11) | Three-Week | 60-minutes, | Three weeks to about one month after the end |
| | | follow-up | then home | of the six-week course, above, the patient will |
| | | post-course | | optionally attend for a 60-minute non-drug |
| | | | | follow-up appointment. The clinical team will |
| | | | | evaluate whether further courses of ketamine |
| | | | | therapy are indicated. |

In embodiments, an exemplary KAP treatment regimen proceeds as outlined in TABLE 16, and this exemplary KAP treatment regimen can be further elucidated as follows.

In embodiments, all therapeutic sessions (both drug and non-drug assisted) will take place in a quiet, comfortable setting. In preferred embodiments, the room will be private and there will be no interruptions from other people throughout the clinical session. Sessions may take place at any such location conducive for KAP, including both home and clinic settings.

In embodiments, prior to each drug-assisted session, a patient will have previously undergone non-drug preparation psychotherapy sessions. The psychotherapy treatment may be any suitable psychotherapy technique such as described herein. On dosing days, in embodiments, the patient may be advised to either fast or have only a light breakfast prior to arriving at the clinic. In embodiments, the attendant doctor may write the prescription for the day's dose of ketamine which, in embodiments, may have already been decided in collaboration with the patient at the preceding non-drug session or sessions (in embodiments including at least one preceding non-drug session) and will be part of an overall agreed titration plan according to the patient's bespoke clinical needs.

In embodiments, the patient may then be settled onto a bed and made comfortable under a blanket. In embodiments, one or more aids may be utilised while the patient is undergoing the dosing session, including music. In embodiments, music may be played throughout the session via headphones, using specifically chosen playlists (Kaelen 2018).

In embodiments, the therapist may first spend a short duration of time, e.g., about 20-30 minutes in talking therapy or another psychotherapeutic technique with the patient. In embodiments, the talking therapy may include discussing the forthcoming administration of the ketamine composition and psychological issues pertaining to the patient. In embodiments, the patient may then lie back with eye shades on in preparation of receiving ketamine.

In embodiments, the attendant nurse may prepare an IM injection of ketamine as follows: (a) using aseptic technique (wearing gloves, clean environment etc.); (b) using a high concentration preparation of ketamine for injection, including 500 mg/5 mL (or, alternatively, 500 mg/10 mL); (c) exposing the upper arm (deltoid muscle) and cleaning the skin with a single alcohol wipe; and (d) using a wider-bore needle fitted to a 1 or 2 mL syringe: draw up the appropriate dose of ketamine liquid for injection from the bottle of ketamine for injection. Broadly, the volume of liquid ketamine injected IM will vary depending on the dose required and the concentration of the ketamine solution (e.g. whether the 500 mg/10 mL bottle or the 500 mg/5 mL bottle). It will be appreciated that dosing will generally be as described herein, such as by reference to TABLE 1, or as otherwise within the general knowledge of the art. In embodiments, the injection is delivered into the deltoid muscle, and the skin is wiped with a swab (if any blood is present).

In other embodiments, another disclosed ketamine composition is administered, such as IN ketamine (including IN esketamine), a ketamine lozenge, HNK, norketamine, etc.

In embodiments, the attendant nurse may remain present in order to assess that no acute adverse reaction has occurred (e.g., anaphylactic shock). If needed, emergency procedures and equipment (e.g., defibrillator, EpiPen) are available on site (and the nurse may refer to relevant SOPs). At this time, the attendant nurse may leave the room. In embodiments, the attendant therapist will remain with the patient throughout the entire ketamine-assisted session. In embodiments, the therapist will provide psychological support, talking therapy, or one or more other psychotherapeutic techniques during the session.

Where racemic IM ketamine is admistered, the drug effects may peak at approximately 30-minutes and decline after a further 30-60 minutes. In other embodiments, depending on the ketamine composition administered, drug effects may peak and decline at other time points known to those of skill. In embodiments, after about two hours the patient may be able to sit up with support from the attendant therapist, acclimatise, then leave the therapy room. In embodiments, the patient may then remain on-site in the clinic recovery suite. When the patient feels ready to leave, the patient may, in embodiments, be seen by the attendant nurse. If the attendant nurse deems the patient able to leave, the patient may leave the clinic accompanied in some embodiments by a pre-assigned designated person.

### KAP Treatment Regimen—Two Ketamine Administration Sessions

In another exemplary embodiment of a KAP treatment regimen, a patient may complete six total therapy sessions during a four-week treatment phase. In embodiments, the KAP treatment regimen includes one 60-minute preparatory psychotherapy session, and two half-day KAP sessions which, in embodiments, are each followed by a 60-minute non-drug integration psychotherapy session (and then optional 60-minute follow-up sessions). This exemplary course of six sessions can be understood further by reference to TABLE 17 below.

TABLE 17
Exemplary Four-Week KAP Treatment Regimen

| | | | Time | |
|---|---|---|---|---|
| Week | Session | Activity: | Required: | Comments: |
| 1 | 1 | Prep session | 60-minutes, | Non-drug assisted preparation psychotherapy is |
| | | | then home | used to further discuss the patient's psychological |
| | | | | issues and establish their intention for the |
| | | | | therapeutic course. |
| 2 | 2 | K1 IM | Half-Day, | Suicide clinical risk assessment is carried out. |
| | | dose then | then home | Brief psychotherapy is given before, during and |
| | | home | | the dose, as tolerated by the patient. |
| 2 | 3 | Integration | 60-minutes, | Non-drug integration to discuss the material that |
| | | Day after | then home | emerged during yesterday's session. |
| | | K1 | | Decision is made in collaboration with the patient |
| | | | | about progressing to a higher dose for the next |
| | | | | session. |
| 3 | 4 | K2 IM | Half-Day, | Suicide clinical risk assessment is carried out. |
| | | dose then | then home | Brief psychotherapy catch-up before dosing. |
| | | home | | Patient is kept comfortable, with minimal therapist |
| | | | | intervention, until the acute effects wear off. Then |
| | | | | brief psychotherapy and a period of gentle, |
| | | | | supported recovery before the patient is allowed to |
| | | | | go home. |
| 3 | 5 | Integration | 60-minutes, | Non-drug integration to discuss the material that |
| | | Day after | then home | emerged during yesterday's session. Decision is |
| | | K2 | | made in collaboration with the patient about |
| | | | | progressing to a higher dose for the next session. |
| 4 | 6 | Final | 60-minutes, | Non-drug psychotherapy session to further |
| | | Integration | then home | explore and integrate the previous two drug |
| | | session | | experiences. |
| 7 | 7 | Three-Week | 60-minutes, | Three weeks to about one month after the end of |
| | | follow-up | then home | the four-week course, above, the patient will |
| | | post-course | | optionally attend for a 60-minute non-drug |
| | | | | follow-up appointment. The clinical team will |
| | | | | evaluate whether further courses of ketamine |
| | | | | therapy are indicated. |

In embodiments, an exemplary KAP treatment regimen proceeds as outlined in TABLE 17, and by further reference to the text accompanying TABLE 17 above, with such modifications as will be readily appreciated to those of skill based on the teachings herein.

It will be readily appreciated that many modifications may be made to the exemplary KAP treatment regimens described in TABLE 16 and TABLE 17 and accompanying text, and that such modifications will be within the ability of those of ordinary skill, by reference to the teachings herein in combination with the general knowledge in the art. Each KAP treatment regimen also will be understood to be modifiable in individual aspects, for example the choice of a minimally invasive method of administration (intramuscular or IM) could be replaced with intravenous (IV) infusion, or with subcutaneous infusion, or intranasal, oral, buccal, or sublingual administration, or another method of administration or drug delivery such as described above or known in the art, and all patients undergoing courses of KAP treatment could receive one or more specific psychotherapeutic techniques, such as desired by the patient or therapist, and could receive more or less supportive psychotherapy, or receive additional monitoring of response and effects, as well as reduced psychotherapy, support, and/or monitoring, depending on the specific embodiments, the scope of such embodiments understood by reference to the teachings herein and the appended claims.

In embodiments, outcomes for a patient in a KAP treatment regimen will be assessed once at 3-4 weeks after the final integration session (illustrated as Session 11 in the six-week KAP course outlined in TABLE 16, and Session 7 in the four-week KAP course outlined in TABLE 17), and/or any of 3 months, 4 months, 5 months, 6 months, 7 months, 8 months, 9 months, 10 months, 11 months, 1 year, 1.5 years, and 2 years after the baseline visit.

In embodiments, a follow-up visit after the initial therapeutic course takes place during Week 9 (Session 11) of the six-week KAP treatment regimen. At this first follow-up visit (“outcome review” or “evaluation” session), if the patient has decided, and if it is deemed clinically indicated, to undergo further sessions, this can be planned. In embodiments, further sessions may be offered as a repeat of another six-week course, as in TABLE 16. Or the patient may be offered a four-week “booster” course, as in TABLE 17.

It will be appreciated that when undergoing second courses, patients may be able to move more quickly through the titration towards higher doses, as clinically indicated.

In embodiments, means of performing follow-up visits and gathering follow-up data (e.g., to measure therapeutic effect) include in-person visits, telemedicine and other virtual visits, phone calls, automated inquiries and check-ins (e.g., email questionnaires, mobile apps, etc.), and the like. Accordingly, it will be appreciated that a “visit” need not be an in-person or face-to-face visit with a member of the therapy team, or another clinician.

### Example 14: Open Label within-Subject Safety and Tolerability Feasibility Study of KAP in Subjects with Behavioural Addictions

In this Example is described a study to investigate the use of ketamine to treat behavioural addictions. In the study of this Example, subjects will receive a six-week course of abstinence-based therapy. Subjects will receive four sessions with ketamine, with dosage selected in the manner described above. Psychological support, including psychotherapeutic techniques as disclosed herein, will be provided before, during, and after each session.

Safety and tolerability will be assessed alongside psychological and physiological outcome measures. Compulsive or problematic behaviours characteristic of the behavioural addiction (e.g., gambling, gaming, compulsive sexual behaviour, compulsive buying, compulsive technology use, stealing, fire starting, compulsive internet use, problematic pornography consumption, and binge eating), mental well-being, and functioning data will be collected for up to one year following the baseline visit.

The study of this Example will demonstrate that ketamine treatment is well-tolerated by all subjects. No unexpected adverse events will be observed. Psychosocial functioning will improve across the cohort. At nine months after the baseline visit the quantity of time spent engaging in a compulsive behaviour by subjects will compare favorably to previous observational studies with similar populations of people with the same disorder. Such results, in view of the further description and disclosures provided herein, will additionally demonstrate the therapeutic value of a KAP treatment regimen for behavioural addictions.

### Methods

This will be an open label within-subject safety and tolerability feasibility study, in 14 subjects aged 18-65 with a behavioural addiction. All subjects will receive KAP. Main outcome measures will be the number of subjects completing the regimen and adverse events.

Secondary outcome measures will include changes in: (1) frequency of the compulsive or problematic behaviour, (2) drive or urge to engage in the compulsive or problematic behaviour, (3) abstinence from the compulsive or problematic behaviour, (4) relapse into the compulsive or problematic behaviour, (5) severity of symptoms of the compulsive or problematic behaviour, (6) time spent on the compulsive or problematic behaviour; (7) quality of life, including subjective sleep; and (8) psychosocial functioning.

Subjects also will be assessed to determine: (1) prescribed medication use; (2) number and frequency of recreational drugs used (if any); and (3) cravings relating to ketamine; and (4) any subsequent use of illicit ketamine.

Assessments will be done at 3, 6, 9, and optionally 12 or more months after baseline.

Subjects with a primary diagnosis of a behavioural addiction who are seeking to treat their addiction (with or without medical assistance) will be recruited. The sample size will be based on a power calculation of required group size to adequately test the hypothesis.

Subjects will receive a six-week course of abstinence-based therapy comprising 10 psychotherapy sessions (such as described in further detail above). On four of these sessions (as in TABLE 16) subjects will be dosed with open-label ketamine during a half-day assisted therapy session. In an alternative aspect, reflecting an alternative embodiment such as described above, subjects will receive six therapy sessions (as in, for example, TABLE 17).

On each dosing session, subjects will receive an IM dose of ketamine with appropriate dosage selected in the manner as described above. Other sessions will comprise one hour psychotherapy sessions, which employ psychotherapeutic techniques including aspects of Motivational Interviewing and “third wave” cognitive-behavioural approaches.

### Trial Procedures

Behavioural addictions will be identified using the DSM-5 diagnostic criteria for the behavioural addiction, the ICD-11, or any other such diagnostic measure known in the art. Screening will comprise of written informed consent, an evaluation of the patient's physical and mental health background, a psychiatric interview (MINI), and assessments of depression and anxiety severity using the PHQ-9 and GAD-7 questionnaires. Severity of the behaviour disorder will be established using the appropriate diagnostic criteria for mild, moderate, and severe cases. Subjects will receive a thorough physical health check comprising an electrocardiogram (ECG), routine blood tests, blood pressure, heart rate, and physical examination. Following screening, a further “baseline” visit will verify eligible subjects.

Subsequently, eligible subjects enter the six-week (or four-week) KAP treatment regimen. This will entail weekly 60-minute outpatient non-drug psychotherapy sessions, delivered by two clinicians. In the six-week course, dosing with ketamine will occur four times. Physiological changes and the intensity of the acute psychoactive effects of ketamine will be measured throughout the ketamine-assisted session. Acute anxiety will be managed primarily psychologically, but sedative medication (oral lorazepam) will be available.

Subjects will be seen the day after each KAP session for an integration psychotherapy session, and then telephoned daily for six days to assess changes to mood, suicidal risk factors (using C-SSRS) and quality of sleep (using the Leeds Sleep questionnaire). Following the end of the six week (or four week) KAP treatment regimen subjects will carry out additional follow-up questionnaires, with a first at three weeks to about a month after the final integration session (see TABLES 16 and 17 above). They will then be seen again at 3, 6 and 9 months (from baseline) for longer-term follow-up data collection, and optionally again at one year and/or at greater than one year from baseline.

### Data Analysis

All data will be recorded on paper case report forms (CRFs) and then digitised into spreadsheets. Analysis and graphing will be performed using GraphPad (Prism) or MS Excel.

### Results

Severity of behavioural addiction criteria at screening and baseline: As per the inclusion criteria, all eligible subjects will score above the diagnostic threshold on the DSM-5 diagnostic criteria, or applicable diagnostic measure, for a behavioural addiction.

Physiological and tolerability effects during ketamine sessions: All subjects will receive KAP. All tested physiological parameters will remain within normal limits for all KAP sessions. No subjects will experience sustained abnormal physiological disturbance, symptomatic experiences of raised blood pressure, heart rate, or temperature or any other adverse events during KAP sessions. No medical interventions will be required in respect of these or any other physiological events during KAP sessions.

Subjects will give their own subjective score (0-10) of whether they felt Drug Effects, and therapists will record their own objective score of how “altered” each subject appeared. There will be no significant difference between Subjects' and therapists' Drug Effects scores. By the end of each KAP session all drug effects will have returned to baseline.

Changes in behaviour: Data will be collected with respect to the frequency and intensity of a compulsive or problematic behaviour in the month before the “Baseline” screening, throughout the six or four week KAP treatment regimen and for up to nine months after baseline, and subjects will reduce their compulsive or problematic behaviour.

Comparisons will be made for the prevalence of average time spent engaging in the compulsive or problematic behaviour per week between each respective time point of 3, 6 and 9 months following the final therapy session. Appropriate statistical analyses will be executed on the resulting figures via Pearsons correlation coefficients to identify the associated change between each point of data collection and baseline. The differences will then be assessed via Fisher r-z transformation (Lowry, 2001-2022) to test the hypothesis that KAP compared to placebo-assisted psychotherapy is effective in this population of subjects with behavioural addiction.

Other mental health measures and quality of life measures: Brief assessments of mood and anxiety will be made at screening, baseline, after the KAP treatment regimen and at 3, 6, and 9-month follow-ups, using the PHQ-9 and GAD-7 rating scales respectively. Scores will demonstrate a reduction in both anxiety and depression after screening and baseline timepoints, followed by a transient rise in anxiety and depression scores 3 months after baseline and a further reduction at 6 months and a moderate rise again at 9 months post baseline. A variety of further data will be collected, including changes to the quality of sleep, quality of life measures and changes to compassion and empathy scales.

Suicidality: Subjects will undergo the C-SSRS rating scale at screening, baseline, throughout the KAP treatment regimen, after each ketamine session and at 3, 6, and 9 month follow-up visits. No subjects will report current suicidal ideation, intent or plans or self-harm behaviour during the course of the study.

Adverse Events: The acute effects of KAP will be well-tolerated by subjects. No unexpected adverse events will occur. No subjects will report any desire to use illicit ketamine following receiving clinical ketamine as part of this trial. No psychotic symptoms will be observed in any subjects.

### Analysis

Through such examples, KAP is demonstrated to be useful in treating behavioural addictions, in the manner in which such treatment is described herein.

### Example 15: Randomised Double-Blind Between-Subject Controlled Study of KAP in Subjects with Behavioural Addictions

This Example describes a randomised, double-blind controlled study of the efficacy of KAP for the treatment of behavioural addiction. This is a randomised between-subject controlled study, in subjects with a behavioural addiction. Half of the subjects will receive KAP and the other half will receive placebo-assisted psychotherapy.

This study will show that KAP is an effective treatment for behavioural addiction, is superior to placebo-assisted psychotherapy, and will result in subjects having a reduction in at least one symptom of the behavioural addiction and an improvement in overall quality of life.

Outcomes regarding (1) frequency of the compulsive or problematic behaviour, (2) drive or urge to engage in the compulsive or problematic behaviour, (3) abstinence from the compulsive or problematic behaviour, (4) relapse into the compulsive or problematic behaviour, (5) severity of symptoms of the compulsive or problematic behaviour, (6) time spent on the compulsive or problematic behaviour; (7) quality of life, including subjective sleep; and (8) psychosocial functioning will be evaluated.

Subjects will also be assessed to determine: (1) prescribed medication use; (2) number and frequency of recreational drugs used (if any); and (3) cravings relating to ketamine; and (4) any subsequent use of illicit ketamine. In embodiments, treatment according to the disclosed methods will result in no change to any one or more of, will result in a reduction in one or more of, will result in an elimination of one or more of, will result in no initiation of any one or more of, or will result in no increase in any one of more of: (1) prescribed medication use; (2) number and frequency of recreational drugs used (if any); and (3) cravings relating to ketamine; and (4) any subsequent use of illicit ketamine.

The main outcome measure will be efficacy, which will be measured in time spent in the compulsive or problematic behaviour at 3, 6, and 9 months post baseline, and at one year.

Here, as also in Examples above, the study will adhere to the principles outlined in the Medicines for Human Use (Clinical Trials) Regulations 2004 (SI 2004/1031), amended regulations (SI 2006/1928) and the International Conference on Harmonisation Good Clinical Practice (ICH GCP) guidelines, the Data Protection Act, and such other regulatory requirements as appropriate and as would be understood to those of ordinary skill in the art.

A population of adult subjects with a primary diagnosis of a behavioural addiction, aged between 18-65, and who meet the inclusion and exclusion criteria (as described herein or known in the art) will be recruited (in a number sufficient to generate statistically significant results). Subjects will remain in the study for a duration of about 14 months.

As further described in the other Examples herein, subjects will undergo a six week (or four week) KAP treatment regimen, comprising psychotherapy sessions as described above. Subjects are randomised to either placebo or ketamine. On four (or two) psychotherapy sessions subjects will be given either placebo (e.g., saline solution) or an active therapeutic dose of ketamine during half-day KAP sessions. Other sessions will comprise non-drug-assisted 60-minute psychotherapy sessions.

The sessions in general will be as described above, and reference is thus made to TABLES 16 and 17 and the corresponding description. The follow-up period will last from the final integration session (or, if discontinued treatment, from two weeks post the final psychotherapy session) to the final follow-up visit (one year post baseline). Subjects will be followed up at 3, 6, and 9 months from the date of baseline, and at one year.

Results are obtained as above, and through this Example, KAP is demonstrated to be useful in treating a behavioural addiction, and to have such other benefits as claimed.

Accordingly, in some embodiments, the ketamine compositions of the invention (and their use in the disclosed methods) are used to improve the symptoms of a behavioural addiction. The symptoms of the behavioural addiction to be treated shall be able to be determined by one of skill, by reference to the general knowledge of the art on that disorder, for example by reference to the DSM-5 or the ICD-11, or as described herein. The disclosed compositions and methods of their use will be understood as improving such symptoms.

In embodiments, the disclosed ketamine compositions (and their use in the disclosed methods) will produce a therapeutic effect, and such effect may be (1) a reduction in a behaviour, (2) a reduction in drive or urge to engage in the behaviour, (3) a promotion of abstinence from the behaviour, (4) a prevention of relapse into the behaviour, (5) a delay in resumption of the behaviour, (6) a reduction in at least one symptom of a behavioural addiction, (7) an elimination of at least one symptom of the behavioural addiction, (8) and a reduction in classification of the behavioural addiction.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Hence, the scope of the invention should be defined solely by the following claims and their equivalents.

### REFERENCES

1. APA 2013. Diagnostic and statistical manual of mental disorders: DSM-5. Washington D.C.: American Psychiatric Association.
2. Asami, Y., Nomura, K., Shimada, H., Ohishi, H., & Ohishi, M. (2020). Validity and reliability of the Japanese version of the kleptomania symptom assessment scale: A comparison between individuals with kleptomania and prisoners in Japan. Comprehensive psychiatry, 96, 152133. https://doi.org/10.1016/j.comppsych.2019.152133
3. Atalayer, D. et al. (2013). Ghrelin and eating disorders. Progress in neuropsychopharmacology & biologicalpsychiatry, vol. 40: 70-82. doi:10.1016/j.pnpbp.2012.08.011
4. Beck A T, Steer R A, Brown G K. Manual for The Beck Depression Inventory Second Edition (BDI-II). San Antonio: Psychological Corporation; 1996.
5. Bierer, L. M. et al., Intergenerational Effects of Maternal Holocaust Exposure on FKBP5 Methylation, American Journal ofPsychiatry 2020 177:8, 744-753.
6. Bloch M H, Wasylink S, Landeros-Weisenberger A, et al. Effects of ketamine in treatment-refractory obsessive-compulsive disorder. Biol Psychiatry. 2012; 72(11):964-970.
7. Böthe, Beáta & Tóth-Király, István & Zsila, Ágnes & Griffiths, Mark & Demetrovics, Zsolt & Gábor, Orosz. (2017). The Development of the Problematic Pornography Consumption Scale (PPCS). The Journal of Sex Research. 55. 10.1080/00224499.2017.1291798.
8. Bouju et al., Journal of Gambling Studies, 2014; 30(2):349-367.
9. Busner, J., & Targum, S. D. (2007). The clinical global impressions scale: applying a research tool in clinical practice. Psychiatry (Edgmont (Pa.: Township)), 4(7), 28-37.
10. Buysse et al., Psychiatry Res., 1989; 28(2):193-213.
11. Calado F, Griffiths M. D. Problem gambling worldwide: an update and systematic review of empirical research (2000-2015). J Behav Addict. (2016) 5:592-613.10.1556/2006.5.2016.073.
12. Cash, H., Rae, C. D., Steel, A. H., & Winkler, A. (2012). Internet Addiction: A Brief Summary of Research and Practice. Current psychiatry reviews, 8(4), 292-298. https://doi.org/10.2174/157340012803520513
13. Coleman, E., Swinburne Romine, R., Dickenson, J., & Miner, M. H. (in press). Compulsive sexual behaviour inventory—13. In R. R. Milhausen, J. K. Sakaluk, T. D. Fisher, C. M. Davis, W. L. Yarber (Eds.), Handbook of sexuality-related measures. New York, NY: Routledge.
14. Coles, T., Coon, C., DeMuro, C., McLeod, L., & Gnanasakthy, A. (2014). Psychometric evaluation of the Sheehan Disability Scale in adult patients with attention-deficit/hyperactivity disorder. Neuropsychiatric disease and treatment, 10, 887-895. https://doi.org/10.2147/NDT.S55220
15. Chamberlain, S. R., Lochner, C., Stein, D. J., Goudriaan, A. E., van Holst, R. J., Zohar, J., & Grant, J. E. (2016). Behavioural addiction—a rising tide?. European Neuropsychopharmacology, 26(5), 841-855.
16. Chang, L. et al. Comparison of antidepressant and side effects in mice after intranasal administration of (R,S)-ketamine, (R)-ketamine, and (S)-ketamine. Pharm. Biochem. Behav 181, 53-59 (2019).
17. Chen, L., Jiang, X., Wang, Q., Böthe, B., Potenza, M. N., & Wu, H. (2021). The Association between the Quantity and Severity of Pornography Use: A Meta-analysis. Journal of sex research, 1-16. Advance online publication. https://doi.org/10.1080/00224499.2021.1988500
18. Columb, D., Hussain, R., & O'Gara, C. (2020). Addiction psychiatry and COVID-19: impact on patients and service provision. Irish Journal of Psychological Medicine, 37(3), 164-168.)
19. Corkery, J. M., Hung, W. C., Claridge, H., Goodair, C., Copeland, C. S., & Schifano, F. (2021). Recreational ketamine-related deaths notified to the National Programme on Substance Abuse Deaths, England, 1997-2019. Journal of psychopharmacology (Oxford, England), 35(11), 1324-1348. https://doi org/10.1177/02698811211021588
20. Cuncic, A. (2021, January 22). The mini-social phobia inventory screens for Social Anxiety Disorder. Verywell Mind. Retrieved May 24, 2022, from https://www.verywellmind.com/mini-social-phobia-inventory-mini-spin-3024440
21. Dalbudak, E., & Evren, C. (2014). The relationship of Internet addiction severity with Attention Deficit Hyperactivity Disorder symptoms in Turkish University students; impact of personality traits, depression and anxiety. Comprehensive psychiatry, 55(3), 497-503. https://doi.org/10.1016/j.comppsych.2013.11.018
22. Davis, JSAS Catalog of Selected Documents in Psychology, 1980; 10, 85.
23. Dechant, E., Boyle, B., and Ross, R. (2020) Ketamine in a Patient with Comorbid Anorexia and MDD. Journal of Women's Health and Development 3 (2020): 373-375.
24. Eilertsen, T., Hansen, B., Kvale, G., Abramowitz, J. S., Holm, S., & Solem, S. (2017). The Dimensional Obsessive-Compulsive Scale: Development and Validation of a Short Form (DOCS-SF). Frontiers in psychology, 8, 1503. https://doi.org/10.3389/fpsy.2017.01503
25. Escrivá-Martinez, T., Galiana, L., Rodríguez-Arias, M., & Baños, R. M. (2019). The Binge Eating Scale: Structural Equation Competitive Models, Invariance Measurement Between Sexes, and Relationships With Food Addiction, Impulsivity, Binge Drinking, and Body Mass Index. Frontiers in psychology, 10, 530. https://doi.org/10.3389/fpsyg.2019.00530
26. Ezquerra-Romano I, Lawn W, Krupitsky E, Morgan C J A. Ketamine for the treatment of addiction: Evidence and potential mechanisms. Neuropharmacology. 2018 November; 142:72-82.
27. Farmer, C A et al. (2020). Ketamine metabolites, clinical response, and gamma power in a randomised, placebo-controlled, crossover trial for treatment-resistant major depression. Neuropsychopharmacology: official publication of the American College of Neuropsychopharmacology. 45(8), 1398-1404. https://doi.org/10.1038/s41386-020-0663-6
28. Ferris, J. A., & Wynne, H. J. (2001). The Canadian problem gambling index (pp. 1-59). Ottawa, ON: Canadian Centre on Substance Abuse.
29. Feder A, et al. Efficacy of intravenous ketamine for treatment of chronic posttraumatic stress disorder: a randomised clinical trial. JAMA Psychiatry. 2014; 71(6):681-688.
30. Floros G D. Gambling disorder in adolescents: prevalence, new developments, and treatment challenges. Adolesc Health Med Ther 2018; 9:43-51. Published 2018 March 2.
31. Francois, J., Grimm, O., Schwarz, A. et al. Ketamine Suppresses the Ventral Striatal Response to Reward Anticipation: A Cross-Species Translational Neuroimaging Study. Neuropsychopharmacol 41, 1386-1394 (2016).
32. Fong T W. The biopsychosocial consequences of pathological gambling. Psychiatry (Edgmont). 2005; 2(3):22-30.
33. Ford, M., & Håkansson, A. (2020). Problem gambling, associations with comorbid health conditions, substance use, and behavioural addictions: Opportunities for pathways to treatment. PLoS One, 15(1), e0227644.
34. Fukumoto, K., Toki, H., Iijima, M., Hashihayata, T., Yamaguchi, J. I., Hashimoto, K., & Chaki, S. (2017). Antidepressant potential of (R)-ketamine in rodent models: comparison with (S)-ketamine. Journal of Pharmacology and Experimental Therapeutics, 361(1), 9-16.)
35. Ginley, M. K., Zajac, K., Rash, C. J., & Petry, N. M. (2019). Pharmacological treatment of behavioural addictions.
36. Glue P, et al. Ketamine's dose-related effects on anxiety symptoms in patients with treatment refractory anxiety disorders. J Psychopharmacol. 2017; 31(10):1302-1305.
37. Glue P, Neehoff S M, Medlicott N J, Gray A, Kibby G, McNaughton N. Safety and efficacy of maintenance ketamine treatment in patients with treatment-refractory generalised anxiety and social anxiety disorders. J Psychopharmacol. 2018; 32(6):663-667.
38. Gould, T., Highland, J., Zanos, P., Thomas, C., and Morris P. (Mar. 3, 2022). PCT Pub. No. 2022/047256. “Hydroxynorketamine compounds and methods of use thereof”
39. Grady, Sarah E et al. “Ketamine for the treatment of major depressive disorder and bipolar depression: A review of the literature.” The mental health clinician vol. 7, 1 16-23.23 Mar. 2018, doi:10.9740/mhc.2017.01.016
40. Grant, J. E., Schreiber, L. R., & Odlaug, B. L. (2013). Phenomenology and treatment of behavioural addictions. The Canadian Journal of Psychiatry, 58(5), 252-259.
41. Grupski, A. E., Hood, M. M., Hall, B. J., Azarbad, L., Fitzpatrick, S. L., & Corsica, J. A. (2013). Examining the Binge Eating Scale in screening for binge eating disorder in bariatric surgery candidates. Obesity surgery, 23(1), 1-6. https://doi.org/10.1007/sI1695-011-0537-4
42. Hallgren K A, Witkiewitz K. Missing data in alcohol clinical trials: a comparison of methods. Alcohol Clin Exp Res. 2013 December; 37(12):2152-60.
43. Hayes, S., Levin, M., Plumb-Vilardaga, J., Villatte, J. & Pistorello, J. (2013). behaviour Therapy, 44 (2), 180-198.
44. Hodgins et al., Addictive behaviours, 2001; 26:121-128.
45. Hodgins D. C. (2004). Using the NORC DSM Screen for Gambling Problems as an outcome measure for pathological gambling: psychometric evaluation. Addictive behaviours, 29(8), 1685-1690. https://doi.org/10.1016/j.addbeh.2004.03.017
46. Hofmarcher, T., Romild, U., Spångberg, J. et al. The societal costs of problem gambling in Sweden. BMC Public Health 20, 1921 (2020). https://doi.org/10.1186/s12889-020-10008-9
47. Hollander E, Berlin H A (2008). Impulse-control disorders measures. In: AJ Rush, MB First, D Blacker (Eds). Handbook of Psychiatric Measures, Second Edition. Washington, DC, American Psychiatric Publishing, Inc. pp 667-686.
48. Huang, R. Y., Pei, L., Liu, Q., Chen, S., Dou, H., Shu, G., Yuan, Z. X., Lin, J., Peng, G., Zhang, W., & Fu, H. (2019). Isobologram Analysis: A Comprehensive Review of Methodology and Current Research. Frontiers in pharmacology, 10, 1222. https://doi.org/10.3389/fphar.2019.01222
49. Ko, C. H., Chen, S. H., Wang, C. H., Tsai, W. X., & Yen, J. Y. (2019). The Clinical Utility of the Chen Internet Addiction Scale-Gaming Version, for Internet Gaming Disorder in the DSM-5 among Young Adults. International journal of environmental research and public health, 16(21), 4141. https://doi.org/10.3390/ijerph16214141
50. Ionescu D F, et al. A single infusion of ketamine improves depression scores in patients with anxious bipolar depression. Bipolar Disord. 2014; 17(4):438-443.
51. Ivan Ezquerra-Romano I, Lawn W, Krupitsky E, Morgan C J A. (2018) Ketamine for the treatment of addiction: Evidence and potential mechanisms. Neuropharmacology. 2018.
52. Jaiswal et al. (2015). Nanoemulsion: an advanced mode of drug delivery system, Biotech 3(5):123-27.
53. Kaelen, M., Giribaldi, B., Raine, J. et al. The hidden therapist: evidence for a central role of music in psychedelic therapy. Psychopharmacology 235, 505-519 (2018). https://doi.org/10.1007/s00213-017-4820-5
54. Kassinove, Journal of Clinical Psychology, 1998; 54(6), 763-771.
55. Kesici, A., & Tung, N. F. (2018, November 30). The development of the Digital Addiction Scale for the University Students: Reliability and validity study. Universal Journal of Educational Research. Retrieved May 18, 2022, from https://eric.ed.gov/?id=EJ1165430
56. Kim S W, Grant J E, Adson D E, et al. Double-blind naltrexone and placebo comparison study in the treatment of pathological gambling. Biol Psychiatry. 2001; 49:914-921).
57. Konkolÿ Thege B, Woodin E M, Hodgins D C, et al. Natural course of behavioural addictions: a 5-year longitudinal study. BMC Psychiatry 2015; 15:4.
58. Kraus, Shane & Grubbs, Joshua. (2019). Brief Pornography Screener: Identifying problematic pornography use in a U.S. sample.
59. Kroenke et al., J Gen Intern Med., 2001; 16(9):606-13.
60. Kruegel, A. C. (Feb. 10, 2022). U.S. Pub. No. 2022/0041540. “Arylcyclohexylamine derivatives and their use in the treatment of psychiatric disorders”
61. Krupitsky, E. M. (1995) Ketamine psychedelic therapy (KPT) of alcoholism and neurosis. In: Yearbook of the European College for the Study of Consciousness (Leuner, H., ed.), pp. 113-121. Verlag Fur Wissenschaft und Bildung, Berlin.
62. Krupitsky E M, Burakov A M, Dunaevsky I V, Romanova T N, Slavina T Y, Grinenko A Y. Single versus repeated sessions of ketamine-assisted psychotherapy for people with heroin dependence. J Psychoactive Drugs. 2007 March; 39(1):13-9.
63. Kurdi, M., Theerth, K. and Deva, R., 2014. Ketamine: Current applications in anesthesia, pain, and critical care. Anesthesia: Essays and Researches, 8(3), p. 283.
64. Lesieur & Bloom, American Journal of Psychiatry, 1990; 144(9).
65. Lévesque, D., Sévigny, S., Giroux, I., & Jacques, C. (2017). Gambling-Related Cognition Scale (GRCS): Are skills-based games at a disadvantage? Psychology of Addictive behaviours, 31(6), 647-654. https://doi.org/10.1037/adb0000297
66. Lewis M. Addiction and the Brain: Development, Not Disease. Neuroethics. 2017; 10(1):7-18.
67. Leite, Priscilla Lourengo; Filomensky, Tatiana Zambrano; Black, Donald W.; Silva, Adriana Cardoso (2014). Validity and reliability of the Brazilian version of Yale-Brown Obsessive Compulsive Scale-Shopping Version (YBOCS-SV). Comprehensive Psychiatry, 55(6), 1462-1466. doi:10.1016/j.comppsych.2014.04.012
68. Lin et al., PLoS One, 2016; 11(11): e0163010
69. Lin, F., He, Y., Zhang, L., Zhang, M., Zhang, Y., & Wen, C. (2015). Assessment of the effect of ketamine on cytochrome P450 isoforms activity in rats by cocktail method. International journal of clinical and experimental medicine, 8(3), 4335-4341.
70. Lobo, D., Aleksandrova, L., Knight, J. et al. Addiction-related genes in gambling disorders: new insights from parallel human and pre-clinical models. Mol Psychiatry 20, 1002-1010 (2015).
71. Long, C. G., Banyard, E., Fulton, B., & Hollin, C. R. (2014). Developing an assessment of fire-setting to guide treatment in secure settings: the St Andrew's Fire and Arson Risk Instrument (SAFARI). Behavioural and cognitive psychotherapy, 42(5), 617-628. https://doi.org/10.1017/S1352465813000477
72. Lowry, R. (2001-2022). Significance of the difference between two correlation coefficients. Retrieved from http://vassarstats.net/rdiffhtmL
73. Lupi, M., Martinotti, G., Acciavatti, T., Pettorruso, M., Brunetti, M., Santacroce, R., . . . & Di Giannantonio, M. (2014). Pharmacological treatments in gambling disorder: a qualitative review. BioMed research international, 2014).
74. Mills I H, Park G R, Manara A R, Merriman R J. Treatment of compulsive behaviour in eating disorders with intermittent ketamine infusions. QJM. 1998 July; 91(7):493-503. doi: 10.1093/qjmed/91.7.493. PMID: 9797933.
75. Mithoefer, M (2017). A Manual for MDMA-Assisted Psychotherapy in the Treatment of Posttraumatic Stress Disorder. https://maps.ora/research/mdma/mdma-research-timeline/4887-a-manual-for-mdma-assisted-psychotherapy-in-the-treatment-of-ptsd.
76. Monacis, L., Palo, V., Griffiths, M. D., & Sinatra, M. (2016). Validation of the Internet Gaming Disorder Scale—Short-Form (IGDS9-SF) in an Italian-speaking sample. Journal of behavioural addictions, 5(4), 683-690. https://doi.org/10.1556/2006.5.2016.083
77. Morgan. C. J. A., Mofeez, A., Bradner, B., Bromley, L., & Curran, H. V. (2004). Acute effects of ketamine on memory systems and psychotic symptoms in healthy volunteers., Neuropsychopharmacology, 29: 208-218.
78. Morgan C J A, Lawn, W, McAndrew A, Stevens, T, Nutt D J (2017) Tripping up addiction: the use of psychedelic drugs in the treatment of problematic drug and alcohol use. Current Opinion in Behavioural Science 13: 71-76.
79. Muela, I., Navas, J. F., & Perales, J. C. (2021). Gambling-Specific Cognitions Are Not Associated With Either Abstract or Probabilistic Reasoning: A Dual Frequentist-Bayesian Analysis of Individuals With and Without Gambling Disorder. Frontiers in psychology, 11, 611784. https://doi.org/10.3389/fpsyg.2020.611784
80. Mundt, J C et al. (2013). “Prediction of suicidal behaviour in clinical research by lifetime suicidal ideation and behaviour ascertained by the electronic Columbia-Suicide Severity Rating Scale.”. The Journal of clinical psychiatry 74 (9): 887-93.
81. Murrough, J. W., Iosifescu, D. V., Chang, L. C., Al Jurdi, R. K., Green, C. E., Perez, A. M., Iqbal, S., Pillemer, S., Foulkes, A., Shah, A., Charney, D. S., & Mathew, S. J. (2013). Antidepressant efficacy of ketamine in treatment-resistant major depression: a two-site randomised controlled trial. The American journal of psychiatry, 170(10), 1134-1142.
82. Neff, Self and Identity, 2003; 2, 223-250
83. Nowak D E, Aloe A M. The prevalence of pathological gambling among college students: A meta-analytic synthesis, 2005-2013. Journal of Gambling Studies. 2014; 30(4):819-843.
84. Obradovic, M., Sudar-Milovanovic, E., Soskic, S., Essack, M., Arya, S., Stewart, A. J., Gojobori, T., & Isenovic, E. R. (2021). Leptin and Obesity: Role and Clinical Implication. Frontiers in endocrinology, 12, 585887. https://doi.org/10.3389/fendo.2021.585887
85. Olive M F, Cleva R M, Kalivas P W, et al. Glutamatergic medications for the treatment of drug and behavioural addictions. Pharmacol Biochem Behav. 2012; 100(4):801-810.
86. Parker, W. (2021, September 1). What is pornography addiction? Verywell Mind. Retrieved May 18, 2022, from https://www.verywellmind.com/breaking-pornography-addictions-1270612
87. Petry N M, Rehbein F, Gentile D A, Lemmens J S, Rumpf H J, Mossle T, Bischof G, Tao R, Fung D S, Borges G, et al. An international consensus for assessing Internet gaming disorder using the new DSM-5 approach. Addiction. 2014; 109(9):1399-406.
88. Petry N. M., Ginley, M. K., & Rash, C. J. (2017). A systematic review of treatments for problem gambling. Psychology of Addictive behaviours, 31(8), 951.)
89. Pinna, F., Dell'Osso, B., Di Nicola, M., Janiri, L., Altamura, A. C., Carpiniello, B., & Hollander, E. (2015). Behavioural addictions and the transition from DSM-IV-TR to DSM-5. Journal of Psychopathology, 21(4), 380-9.
90. Pontes, H. M., Király, O., Demetrovics, Z., & Griffiths, M. D. (2014). The conceptualisation and measurement of DSM-5 Internet Gaming Disorder: the development of the IGD-20 Test. PloS one, 9(10), e110137. https://doi.org/10.1371/journal.pone.0110137
91. Portmann, S., Kwan, H. Y., Theurillat, R., Schmitz, A., Mevissen, M., & Thormann, W. (2010). Enantioselective capillary electrophoresis for identification and characterization of human cytochrome P450 enzymes which metabolize ketamine and norketamine in vitro. Journal of chromatography. A, 1217(51), 7942-7948. https://doi.org/10.1016/j.chroma.2010.06.028
92. Protocol—sensitivity to punishment sensitivity to reward questionnaire (SPSRQ). PhenX Toolkit. (n.d.). Retrieved May 17, 2022, from https://www.phenxtoolkit.org/protocols/view/540603 #:˜:text=The %20Sensitivity %20 to %20Pun ishment %2FSensitivity, Sensitivity %20 to %20Punishment %20(SP).
93. Raylu, N., & Oei, T. P. (2004). The Gambling Related Cognitions Scale (GRCS): development, confirmatory factor validation and psychometric properties. Addiction (Abingdon, England), 99(6), 757-769. https://doi.org/10.1111/j.1360-0443.2004.00753.x
94. Rodriguez C I, Kegeles L S, Levinson A, et al. Randomised controlled crossover trial of ketamine in obsessive-compulsive disorder: proof-of-concept. Neuropsychopharmacology. 2013; 38(12):2475-2483.
95. Samaha, A. A., Fawaz, M., El Yahfoufi, N., Gebbawi, M., Abdallah, H., Baydoun, S. A., Ghaddar, A., & Eid, A. H. (2018). Assessing the Psychometric Properties of the Internet Addiction Test (IAT) Among Lebanese College Students. Frontiers in public health, 6, 365. https://doi.ora/10.3389/fpubh.2018.00365
96. Schwartz J, Murrough J W, Iosifescu D V. Ketamine for treatment-resistant depression: recent developments and clinical applications. Evid Based Ment Health. 2016; 19(2):35-38.
97. Scolnick, B., Zupec-Kania, B., Calabrese, L., Aoki, C., & Hildebrandt, T. (2020). Remission from Chronic Anorexia Nervosa With Ketogenic Diet and Ketamine: Case Report. Frontiers in psychiatry, 11, 763. https://doi.org/10.3389/fpsyt.2020.00763.
98. Sheehan D V, Lecrubier Y, Sheehan K H, Amorim P, Janavs J, Weiller E, Hergueta T, Baker R, Dunbar G C. (1998) The Mini-International Neuropsychiatric Interview (M.I.N.I.): the development and validation of a structured diagnostic psychiatric interview for DSM-IV and ICD-10. J Clin Psychiatry.; 59 Suppl 20:22-33; quiz 34-57.
99. Shiroma, P. R., Thuras, P., Wels, J. et al. A randomised, double-blind, active placebo-controlled study of efficacy, safety, and durability of repeated vs single subanesthetic ketamine for treatment-resistant depression. Transl Psychiatry 10, 206 (2020).
100. Shonin, E., Van Gordon, W., & Griffiths, M. D. (2014). Mindfulness as a treatment for behavioural addiction. Journal of Addiction Research & Therapy, 5(1).
101. Sih, K., et al., 2011. Ketamine in Adult Emergency Medicine: Controversies and Recent Advances. Annals Pharmacother, 45(12), 1525-1534.
102. Sleep Related Questionnaires, Morningness-Eveningness Questionnaire (MEQ). American Thoracic Society. (2022). Retrieved May 23, 2022, from https://www.thoracic.org/members/assemblies/assemblies/srn/questionaires/meq.php
103. Sloshower, J., Guss, J., Krause, R., Wallace, R., Williams, M., Reed, S. & Skinta, M. Psilocybin-assisted therapy of major depressive disorder using Acceptance and Commitment Therapy as a therapeutic frame. J Contextual Behavioural Science, 15, 12-19.
104. Smith, M. (2021, February 6). Global Assessment of Functioning (GAF) scale. WebMD. Retrieved May 24, 2022, from https://www.webmd.com/mental-health/gaf-scale-facts
105. Sobell, L. C., Agrawal, S., Annis, H. M., et al. (2001). Cross-cultural evaluation of two drinking related assessment instruments: Alcohol Timeline Follow Back and Inventory of Drinking Situations. Substance Use & Misuse, 36, 313-331.
106. Sobell, L. C., Sobell, M. B. (1996). Timeline FollowBack user's guide: A calendar method for assessing alcohol and drug use. Addiction Research Foundation, Toronto, Ontario, Canada; J. Anal. Toxicolo., 2002, 26: 393-400.
107. Spitzer et al., Arch Intern. Med., 2006; 22; 166(10): 1092-7.
108. Srivastava, S., et al. (2015). Safety and efficacy of ketamine infusion in late onset depression, and conversion to treatment response. Indian J Psychiatry, 57(3), 328-329.
109. Sternlicht, L., & Sternlicht, A. (2022). The 6 most common types of technology addiction. Family Addiction Specialist: Addiction Counselor. Retrieved May 23, 2022, from https://www.familyaddictionspecialist.com/blog/the-6-most-common-types-of-technology-addiction
110. Sussman, S., Lisha, N., & Griffiths, M. (2011). Prevalence of the addictions: a problem of the majority or the minority?. Evaluation & the health professions, 34(1), 3-56. https://doi.ora/10.1177/0163278710380124
111. Sykes, M., & Turner, L. (2014). The adult attachment interview & how it changed attachment research history. Psychotherapy Networker. Retrieved May 23, 2022, from https://www.psychotherapynetworker.org/blog/details/17/the-adult-attachment-interview-how-it-changed-attachment
112. Theresa A. Gannon; Emma Barrowcliffe (2012). Firesetting in the general population: The development and validation of the Fire Setting and Fire Proclivity Scales., 17(1), 105-122. doi:10.1348/135532510x523203
113. Toneatto T, Brands B, Selby P. A randomised, double-blind, placebo-controlled trial of naltrexone in the treatment of concurrent alcohol use disorder and pathological gambling. Am J Addict. 2009; 18(3):219-225.
114. Toneatto, T., Pillai, S., & Courtice, E. L. (2014). Mindfulness-enhanced cognitive behaviour therapy for problem gambling: A controlled pilot study. International Journal of Mental Health and Addiction, 12(2), 197-205.
115. Torrubia, R., Avila, C., Molto, J., & Caseras, X. (2001). The Sensitivity to Punishment and Sensitivity to Reward Questionnaire (SPSRQ) as a measure of Gray's anxiety and impulsivity dimensions. Personality and Individual Differences, 31(6), 837-862.
116. Toce-Gerstein, M., Gerstein, D. R., & Volberg, R. A. (2009). The NODS-CLiP: a rapid screen for adult pathological and problem gambling. Journal of gambling studies, 25(4), 541-555. https://doi.org/10.1007/s10899-009-9135-y
117. Wainer, W. I. (Dec. 23, 2021). PCT Pub. No. 2021/255737. “Hydroxynorketamine analogues, compositions comprising same and methods of use thereof”
118. Wang, P. F., Neiner, A., & Kharasch, E. D. (2018). Stereoselective Ketamine Metabolism by Genetic Variants of Cytochrome P450 CYP2B6 and Cytochrome P450 Oxidoreductase. Anesthesiology, 129(4), 756-768. https://doi.org/10.1097/ALN.0000000000002371
119. Watts, R. & Luoma, J. (2020). Journal of Contextual Behavioural Science, 15, 92-102.
120. World Health Organization. (2022). 6B82 Binge eating disorder. World Health Organization. Retrieved May 23, 2022, from https://icd.who.int/browse 11/l-m/en #/http://id.who.int/icd/entity/1673294767
121. World Health Organization. (2022). 6C70 Pyromania. World Health Organization. Retrieved May 23, 2022, from https://icd.who.int/browse 11/l-m/en #/http://id.who.int/icd/entity/1532500290
122. World Health Organization. (2022). 6C71 Kleptomania. World Health Organization. Retrieved May 23, 2022, from https://icd.who.int/browse 11/l-m/en #/http://id.who.int/icd/entity/804604349
123. World Health Organization. (2022). 6C51 Gaming disorder. WHO. Retrieved May 23, 2022, from https://icd.who.int/browse1l/1-m/en #/http://id.who.int/icd/entity/1448597234
124. The World Health Organization. (n.d.) Addictive behaviours. Retrieved from https://www.who.int/health-topics/addictive-behaviours #tab-tab_1).
125. Yamaguchi, J.-ichi, Toki, H., Qu, Y., Yang, C., Koike, H., Hashimoto, K., Mizuno-Yasuhira, A., & Chaki, S. (2018). (2R,6R)-hydroxynorketamine is not essential for the antidepressant actions of (r)-ketamine in mice. Neuropsychopharmacology, 43(9), 1900-1907. https://doi.org/10.1038/s41386-018-0084-y
126. Yang, C., Shirayama, Y., Zhang, Jc. et al. R-ketamine: a rapid-onset and sustained antidepressant without psychotomimetic side effects. Transl Psychiatry 5, 632 (2015).
127. Yehuda, R., et al. (2016). Holocaust Exposure Induced Intergenerational Effects on FKBP5 Methylation. Biological psychiatry, 80(5), 372-380.
128. Yoshitsugu Yanagihara, Satoru Kariya, Michiteru Ohtani, Katsuyoshi Uchino, Takao Aoyama, Yoshikazu Yamamura and Tatsuji Iga. Drug Metabolism and Disposition June 2001, 29 (6) 887-890;
129. YOUNG, KIMBERLY S. (1998). Internet Addiction: The Emergence of a New Clinical Disorder. CyberPsychology & behaviour, 1(3), 237-244. doi:10.1089/cpb.1998.1.237
130. Zanos, P., Moaddel, R., Morris, P. J., Riggs, L. M., Highland, J. N., Georgiou, P., Pereira, E., Albuquerque, E. X., Thomas, C. J., Zarate, C. A., Jr, & Gould, T. D. (2018). Ketamine and Ketamine Metabolite Pharmacology: Insights into Therapeutic Mechanisms. Pharmacological reviews, 70(3), 621-660. https://doi.org/10.1124/pr.117.015198
131. Zhang J. C., Li, S. X. & Hashimoto, K. R(−)-ketamine shows greater potency and longer lasting antidepressant effects than S(+)-ketamine. Pharm. Biochem. Behav. 116, 137-141 (2014).

1-160. (canceled)

161. A method of treating a patient with a behavioral addiction, comprising administering to the patient a therapeutically effective amount of ketamine.

162. The method of claim 161, wherein the method results in a reduction in at least one symptom of the behavioral addiction, an elimination of at least one symptom of the behavioral addiction, or a reduction in the severity of the behavioral addiction.

163. The method of claim 161, wherein the behavioral addiction involves a compulsive behavior or a problematic behavior, and the method results in any of a reduction in the behavior, a reduction in drive or urge to engage in the behavior, a promotion of abstinence from the behavior, a prevention of relapse into the behavior, and a delay in resumption of the behavior.

164. The method of claim 161, wherein the behavioral addiction is any of gambling disorder, gaming disorder, compulsive sexual behavior disorder, compulsive buying-shopping disorder, technology addiction, kleptomania, pyromania, internet addiction, pornography addiction, and binge eating disorder.

165. The method of claim 164, wherein the behavioral addiction is any of gaming disorder, compulsive sexual behavior disorder, compulsive buying-shopping disorder, technology addiction, internet addiction, and pornography addiction.

166. The method of claim 165, wherein the behavioral addiction is gaming disorder.

167. The method of claim 166, wherein the method results in a reduction in at least one symptom of the gaming disorder, an elimination of at least one symptom of the gaming disorder, or a reduction in the severity of the gaming disorder.

168. The method of claim 166, wherein the method results in a reduction in gaming, a reduction in drive or urge to engage in gaming, a promotion of abstinence from gaming, a prevention of relapse into gaming, or a delay in resumption of gaming.

169. The method of claim 165, wherein the behavioral addiction is compulsive sexual behavior disorder.

170. The method of claim 169, wherein the method results in a reduction in at least one symptom of the compulsive sexual behavior disorder, an elimination of at least one symptom of the compulsive sexual behavior disorder, or a reduction in the severity of the compulsive sexual behavior disorder.

171. The method of claim 169, wherein method results in a reduction in compulsive sexual behavior, a reduction in drive or urge to engage in compulsive sexual behavior, a promotion of abstinence from compulsive sexual behavior, a prevention of relapse into compulsive sexual behavior, or a delay in resumption of compulsive sexual behavior.

172. The method of claim 164, wherein the behavioral addiction is gambling disorder.

173. The method of claim 166, wherein the method results in a reduction in at least one symptom of the gambling disorder, an elimination of at least one symptom of the gambling disorder, or a reduction in the severity of the gambling disorder.

174. The method of claim 166, wherein the method results in a reduction in gambling, a reduction in drive or urge to engage in gambling, a promotion of abstinence from gambling, a prevention of relapse into gambling, or a delay in resumption of gambling.

175. The method of claim 161, wherein the ketamine is administered in a dissociative dose.

176. The method of claim 175, wherein the dissociative dose is an amount of at least 50 mg.

177. The method of claim 175, wherein the dissociative dose is an amount of at least 0.7 mg/kg.

178. The method of claim 161, wherein the ketamine is administered sublingually.

179. The method of claim 161, wherein the ketamine is administered together with psychological support or psychotherapy.

180. The method of claim 179, wherein the psychotherapy comprises any of psychosocial or behavioral therapy, cognitive behavioral therapy, interpersonal therapy, contingency management based therapy, community reinforcement approach based therapy, motivational interviewing based therapy, motivational enhancement based therapy, meditation based therapy, acceptance and commitment therapy, dyadic psychotherapy, supportive psychotherapy, attachment-based psychotherapy, behavioral therapy, body psychotherapy, somatic psychotherapy, brief therapy, cognitive analytical therapy, cognitive behavior therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, and mindfulness-based cognitive therapy.
